SEQUENCE LISTING (786 sequences)

SEQ ID NO: 1 - DNA, 169 bp, Unknown
  Enterococcus
cgaggcaatatggctaaagtaattaaagcttttaaacttaaacctgaactgcttattgcagcaatggctaaagtaattaaagcttttaaaacttaaacctgactgtattgatgatcattgtacatcgaaatatctgaataacctcattgacaagattcaccgtgctgag

SEQ ID NO: 2 - DNA, 173 bp, Unknown
  Enterococcus
cgagataatatggttaaagtaattaaaagcttttaaaacttaaaccttgactgctttattgtagcaatggttaaagtaattaaagcttttaaaacttaaacctgactgcattgatgaaccattgtacatcgaaatatctgaataacctcaattgacaagatcacccgtgcgag

SEQ ID NO: 3 - DNA, 169 bp, Unknown
  Enterococcus
cgagataatatggctaaagtaattaaagcttttaaacttaaacctgactgctttattgtagcaatggctaaagtaattaaagcttttaaacttaaacctgaactgtatttgatgatcattgtacatcgaaatatctgaataacctcattgagcaagatcaccgtgcgag

SEQ ID NO: 4 - DNA, 171 bp, Unknown
  Enterococcus
cgagataatatggctaaagtaattaaagcttttaaacttaaacctggactgcttattgtagcaatggctaaagtaattaaagcttttaaacttaaacctgaactgtatttgattgatcattgtacatcgaaatacctgaataacctcattgagcaagatcaccgtgctgag

SEQ ID NO: 5 - DNA, 55 bp, Artificial Sequence
  Synthetic Oligonucleotide
ccatctcatccctgcgtgtctccgactcagnnnnnnggaacgatgagcctccaac

SEQ ID NO: 6 - DNA, 62 bp, Artificial Sequence
  Synthetic Oligonucleotide
ccactacgcctccgctttcctctctatgggcagtcggtgatcagatgttatgctcgcaggtc

SEQ ID NO: 7 - DNA, 83 bp, Artificial Sequence
  Synthetic Oligonucleotide
caagcagaagacggcatacgagatcggtctcggcattcctgctgaaccgctcttccgatctcagatgttatgctcgcaggtnc

SEQ ID NO: 8 - DNA, 84 bp, Artificial Sequence
  Synthetic Oligonucleotide
aatgatacggcgaccaccgagatctacactctttccctacacgacgctcttccgatctnnnnnnggaacgatgagcctccaanc

SEQ ID NO: 9 - DNA, 68 bp, Artificial Sequence
  Synthetic Oligonucleotide
gttggaggctcatcgttcctatattccacaccacttattgatgattacagatgttatgctcgcaggtc

SEQ ID NO: 10 - DNA, 25 bp, Artificial Sequence
  Synthetic Oligonucleotide
accaatagggcttattaaacaactg

SEQ ID NO: 11 - DNA, 28 bp, Artificial Sequence
  Synthetic Oligonucleotide
attgaaatataaattgtaaatcatattc

SEQ ID NO: 12 - DNA, 26 bp, Artificial Sequence
  Synthetic Oligonucleotide
caattgtgtttgtctttgtatccatt

SEQ ID NO: 13 - DNA, 23 bp, Artificial Sequence
  Synthetic Oligonucleotide
tgtctacacattattccacaatc

SEQ ID NO: 14 - DNA, 23 bp, Artificial Sequence
  Synthetic Oligonucleotide
catataccattgttgtggccctg

SEQ ID NO: 15 - DNA, 27 bp, Artificial Sequence
  Synthetic Oligonucleotide
ttgaaatataaattgtaaatcatactc

SEQ ID NO: 16 - DNA, 27 bp, Artificial Sequence
  Synthetic Oligonucleotide
gccttatgtaaccaatatggtttatta

SEQ ID NO: 17 - DNA, 22 bp, Artificial Sequence
  Synthetic Oligonucleotide
actgcaaatcatattcctcaac

SEQ ID NO: 18 - DNA, 19 bp, Artificial Sequence
  Synthetic Oligonucleotide
gctgtcactaggccgccac

SEQ ID NO: 19 - DNA, 22 bp, Artificial Sequence
  Synthetic Oligonucleotide
cgtaccctaaacaccctatatt

SEQ ID NO: 20 - DNA, 24 bp, Artificial Sequence
  Synthetic Oligonucleotide
gtgtctagcaaacatttgttcctt

SEQ ID NO: 21 - DNA, 26 bp, Artificial Sequence
  Synthetic Oligonucleotide
aagccaatatggtttattaaataatt

SEQ ID NO: 22 - DNA, 23 bp, Artificial Sequence
  Synthetic Oligonucleotide
accaatatagtgctgcaacacca

SEQ ID NO: 23 - DNA, 25 bp, Artificial Sequence
  Synthetic Oligonucleotide
catattcatatgcaatatcactttc

SEQ ID NO: 24 - DNA, 20 bp, Artificial Sequence
  Synthetic Oligonucleotide
gctgtcactagaccgccaca

SEQ ID NO: 25 - DNA, 23 bp, Artificial Sequence
  Synthetic Oligonucleotide
ataccctatattgatatgcagac

SEQ ID NO: 26 - DNA, 20 bp, Artificial Sequence
  Synthetic Oligonucleotide
cctgtgcacgttgcaaccaa

SEQ ID NO: 27 - DNA, 22 bp, Artificial Sequence
  Synthetic Oligonucleotide
tgtagttcatattcctccacat

SEQ ID NO: 28 - DNA, 23 bp, Artificial Sequence
  Synthetic Oligonucleotide
gtaatatccaacacagcaggtgt

SEQ ID NO: 29 - DNA, 23 bp, Artificial Sequence
  Synthetic Oligonucleotide
ttgcataggtatttcctcatagc

SEQ ID NO: 30 - DNA, 26 bp, Artificial Sequence
  Synthetic Oligonucleotide
gctgtaatactgtttgtctttctatc

SEQ ID NO: 31 - DNA, 22 bp, Artificial Sequence
  Synthetic Oligonucleotide
taatgtctacacattgttccac

SEQ ID NO: 32 - DNA, 26 bp, Artificial Sequence
  Synthetic Oligonucleotide
aacagttaataatctagaactgccag

SEQ ID NO: 33 - DNA, 22 bp, Artificial Sequence
  Synthetic Oligonucleotide
acgttgtgtttcaggattataa

SEQ ID NO: 34 - DNA, 28 bp, Artificial Sequence
  Synthetic Oligonucleotide
gacatttgtaaataatcaggatatttac

SEQ ID NO: 35 - DNA, 21 bp, Artificial Sequence
  Synthetic Oligonucleotide
gtcagaggtaacaatagagcc

SEQ ID NO: 36 - DNA, 27 bp, Artificial Sequence
  Synthetic Oligonucleotide
aataaatgtctaacaaacatttgctcc

SEQ ID NO: 37 - DNA, 24 bp, Artificial Sequence
  Synthetic Oligonucleotide
ttgcaaccaatatggtttattaaa

SEQ ID NO: 38 - DNA, 28 bp, Artificial Sequence
  Synthetic Oligonucleotide
tctatctaaacttataggatttccatct

SEQ ID NO: 39 - DNA, 27 bp, Artificial Sequence
  Synthetic Oligonucleotide
tatacaggattaccattattatctaat

SEQ ID NO: 40 - DNA, 28 bp, Artificial Sequence
  Synthetic Oligonucleotide
cgttgtaaccaatatggtttattaaata

SEQ ID NO: 41 - DNA, 23 bp, Artificial Sequence
  Synthetic Oligonucleotide
taaactgcaaatcatattcttcc

SEQ ID NO: 42 - DNA, 26 bp, Artificial Sequence
  Synthetic Oligonucleotide
gtccagtttgtataatgcattgtatt

SEQ ID NO: 43 - DNA, 24 bp, Artificial Sequence
  Synthetic Oligonucleotide
aaacacagatgtaggacataatat

SEQ ID NO: 44 - DNA, 23 bp, Artificial Sequence
  Synthetic Oligonucleotide
agtaatagggatgtcctacggca

SEQ ID NO: 45 - DNA, 20 bp, Artificial Sequence
  Synthetic Oligonucleotide
gcctacctccaaacctacac

SEQ ID NO: 46 - DNA, 23 bp, Artificial Sequence
  Synthetic Oligonucleotide
cttggaggttcaattaacatgcg

SEQ ID NO: 47 - DNA, 24 bp, Artificial Sequence
  Synthetic Oligonucleotide
gttatatcattatcaaatgcccac

SEQ ID NO: 48 - DNA, 24 bp, Artificial Sequence
  Synthetic Oligonucleotide
acaaataccattgttgtgtccctg

SEQ ID NO: 49 - DNA, 26 bp, Artificial Sequence
  Synthetic Oligonucleotide
aaatataaattgcaaatcatattcct

SEQ ID NO: 50 - DNA, 21 bp, Artificial Sequence
  Synthetic Oligonucleotide
cacctacacaggcccaaacca

SEQ ID NO: 51 - DNA, 21 bp, Artificial Sequence
  Synthetic Oligonucleotide
gtggtttgcaaccaattaaac

SEQ ID NO: 52 - DNA, 23 bp, Artificial Sequence
  Synthetic Oligonucleotide
ggtgtaatatccaataccgcagg

SEQ ID NO: 53 - DNA, 22 bp, Artificial Sequence
  Synthetic Oligonucleotide
acacttccataggtatttcctc

SEQ ID NO: 54 - DNA, 26 bp, Artificial Sequence
  Synthetic Oligonucleotide
tgcatttggaaattcaaatactgtta

SEQ ID NO: 55 - DNA, 19 bp, Artificial Sequence
  Synthetic Oligonucleotide
gcaacgcacttaaacgttc

SEQ ID NO: 56 - DNA, 27 bp, Artificial Sequence
  Synthetic Oligonucleotide
catacatgtttcaggaattgatagtaa

SEQ ID NO: 57 - DNA, 23 bp, Artificial Sequence
  Synthetic Oligonucleotide
cattatcatatgcccattgtatc

SEQ ID NO: 58 - DNA, 25 bp, Artificial Sequence
  Synthetic Oligonucleotide
caaataccattattgtgtccctgag

SEQ ID NO: 59 - DNA, 27 bp, Artificial Sequence
  Synthetic Oligonucleotide
ctgaaatataaattgtaaatcaaattc

SEQ ID NO: 60 - DNA, 26 bp, Artificial Sequence
  Synthetic Oligonucleotide
tctatctaaacttacaggattcccat

SEQ ID NO: 61 - DNA, 26 bp, Artificial Sequence
  Synthetic Oligonucleotide
ttatttaattgatatacaggattacc

SEQ ID NO: 62 - DNA, 26 bp, Artificial Sequence
  Synthetic Oligonucleotide
aatttccttccagtttgtataatcca

SEQ ID NO: 63 - DNA, 20 bp, Artificial Sequence
  Synthetic Oligonucleotide
cccacatgtacttcccatac

SEQ ID NO: 64 - DNA, 23 bp, Artificial Sequence
  Synthetic Oligonucleotide
gttacaggactaaagggtgttcc

SEQ ID NO: 65 - DNA, 19 bp, Artificial Sequence
  Synthetic Oligonucleotide
tccgtggcaacaactttgg

SEQ ID NO: 66 - DNA, 22 bp, Artificial Sequence
  Synthetic Oligonucleotide
aaacaatgcctgtgctgtctct

SEQ ID NO: 67 - DNA, 19 bp, Artificial Sequence
  Synthetic Oligonucleotide
gtattgccatacccgctgt

SEQ ID NO: 68 - DNA, 24 bp, Artificial Sequence
  Synthetic Oligonucleotide
ctgcccgtttataatgtctacaca

SEQ ID NO: 69 - DNA, 22 bp, Artificial Sequence
  Synthetic Oligonucleotide
tgtttgcaggtccatataatac

SEQ ID NO: 70 - DNA, 23 bp, Artificial Sequence
  Synthetic Oligonucleotide
tattagtgtctgccaattgtgca

SEQ ID NO: 71 - DNA, 21 bp, Artificial Sequence
  Synthetic Oligonucleotide
caatttgcttccaatcacctc

SEQ ID NO: 72 - DNA, 24 bp, Artificial Sequence
  Synthetic Oligonucleotide
caggagttgttgtagaagaggaag

SEQ ID NO: 73 - DNA, 22 bp, Artificial Sequence
  Synthetic Oligonucleotide
atacctccataggtatttcctc

SEQ ID NO: 74 - DNA, 27 bp, Artificial Sequence
  Synthetic Oligonucleotide
cattatcatatgcccattgtatcattt

SEQ ID NO: 75 - DNA, 21 bp, Artificial Sequence
  Synthetic Oligonucleotide
tgtatccattgtcccattgtc

SEQ ID NO: 76 - DNA, 27 bp, Artificial Sequence
  Synthetic Oligonucleotide
aaataaatgtctaacaaacatttgctc

SEQ ID NO: 77 - DNA, 23 bp, Artificial Sequence
  Synthetic Oligonucleotide
aaataccattattatggccttgt

SEQ ID NO: 78 - DNA, 24 bp, Artificial Sequence
  Synthetic Oligonucleotide
gagtggtatctaccacagtaacaa

SEQ ID NO: 79 - DNA, 24 bp, Artificial Sequence
  Synthetic Oligonucleotide
agcatgaatatatgtcataacttc

SEQ ID NO: 80 - DNA, 24 bp, Artificial Sequence
  Synthetic Oligonucleotide
accaaagccagtatcaaccatatc

SEQ ID NO: 81 - DNA, 22 bp, Artificial Sequence
  Synthetic Oligonucleotide
gtctaacaaacatttgttccct

SEQ ID NO: 82 - DNA, 25 bp, Artificial Sequence
  Synthetic Oligonucleotide
gcatcatctaataatgctaccttgg

SEQ ID NO: 83 - DNA, 24 bp, Artificial Sequence
  Synthetic Oligonucleotide
actgtcactctactatgtaaatac

SEQ ID NO: 84 - DNA, 28 bp, Artificial Sequence
  Synthetic Oligonucleotide
gatatacctgttctataccagtataatg

SEQ ID NO: 85 - DNA, 22 bp, Artificial Sequence
  Synthetic Oligonucleotide
tgaaatgccatatcactttcat

SEQ ID NO: 86 - DNA, 22 bp, Artificial Sequence
  Synthetic Oligonucleotide
ctggcaaataattgttcccggc

SEQ ID NO: 87 - DNA, 24 bp, Artificial Sequence
  Synthetic Oligonucleotide
ccagtatggcttattaaatagttg

SEQ ID NO: 88 - DNA, 26 bp, Artificial Sequence
  Synthetic Oligonucleotide
attaaactcattccaaagcatgattt

SEQ ID NO: 89 - DNA, 24 bp, Artificial Sequence
  Synthetic Oligonucleotide
aatacttgtaggatttccatctaa

SEQ ID NO: 90 - DNA, 23 bp, Artificial Sequence
  Synthetic Oligonucleotide
aatccatagctccaaaccctgta

SEQ ID NO: 91 - DNA, 24 bp, Artificial Sequence
  Synthetic Oligonucleotide
gtttattataatagtgcctagcaa

SEQ ID NO: 92 - DNA, 21 bp, Artificial Sequence
  Synthetic Oligonucleotide
cctgtgcacgttgtaaccagt

SEQ ID NO: 93 - DNA, 28 bp, Artificial Sequence
  Synthetic Oligonucleotide
gaaatataaattgtaattcatattcttc

SEQ ID NO: 94 - DNA, 26 bp, Artificial Sequence
  Synthetic Oligonucleotide
acatataccattattgtgtccttgtg

SEQ ID NO: 95 - DNA, 24 bp, Artificial Sequence
  Synthetic Oligonucleotide
acacaattgaaatataaattgcac

SEQ ID NO: 96 - DNA, 23 bp, Artificial Sequence
  Synthetic Oligonucleotide
catcctcatcctctgagttgtcc

SEQ ID NO: 97 - DNA, 21 bp, Artificial Sequence
  Synthetic Oligonucleotide
cctagtgtacccataagcaac

SEQ ID NO: 98 - DNA, 28 bp, Artificial Sequence
  Synthetic Oligonucleotide
taatattagacaaacctgttctatacca

SEQ ID NO: 99 - DNA, 24 bp, Artificial Sequence
  Synthetic Oligonucleotide
ctgtgcatatttatatgctatgtc

SEQ ID NO: 100 - DNA, 27 bp, Artificial Sequence
  Synthetic Oligonucleotide
aaataattgattatgccaacaaatacc

SEQ ID NO: 101 - DNA, 26 bp, Artificial Sequence
  Synthetic Oligonucleotide
gaattcatagaatgtatatatgtcat

SEQ ID NO: 102 - DNA, 22 bp, Artificial Sequence
  Synthetic Oligonucleotide
gctgtcattagtacgccacaaa

SEQ ID NO: 103 - DNA, 22 bp, Artificial Sequence
  Synthetic Oligonucleotide
ccctatactgaaaggcagatac

SEQ ID NO: 104 - DNA, 26 bp, Artificial Sequence
  Synthetic Oligonucleotide
atatctttccaattaccaccatcatt

SEQ ID NO: 105 - DNA, 25 bp, Artificial Sequence
  Synthetic Oligonucleotide
ctagttgaatttacatacgaaataa

SEQ ID NO: 106 - DNA, 21 bp, Artificial Sequence
  Synthetic Oligonucleotide
atatgcccactgcaccatgtc

SEQ ID NO: 107 - DNA, 20 bp, Artificial Sequence
  Synthetic Oligonucleotide
gctttatccactcagccatt

SEQ ID NO: 108 - DNA, 23 bp, Artificial Sequence
  Synthetic Oligonucleotide
tatataaaccaaggcgtgccaca

SEQ ID NO: 109 - DNA, 19 bp, Artificial Sequence
  Synthetic Oligonucleotide
agcaggcctatgtaatgca

SEQ ID NO: 110 - DNA, 24 bp, Artificial Sequence
  Synthetic Oligonucleotide
agatatacggtattgtcactaggc

SEQ ID NO: 111 - DNA, 23 bp, Artificial Sequence
  Synthetic Oligonucleotide
tgcaccctaaatactctatattg

SEQ ID NO: 112 - DNA, 21 bp, Artificial Sequence
  Synthetic Oligonucleotide
caacacctacacaggcccaga

SEQ ID NO: 113 - DNA, 21 bp, Artificial Sequence
  Synthetic Oligonucleotide
ggtacacagccaataatacac

SEQ ID NO: 114 - DNA, 28 bp, Artificial Sequence
  Synthetic Oligonucleotide
ccatctaataggtttctcatatatgtat

SEQ ID NO: 115 - DNA, 22 bp, Artificial Sequence
  Synthetic Oligonucleotide
agttcatatactgcattcccat

SEQ ID NO: 116 - DNA, 28 bp, Artificial Sequence
  Synthetic Oligonucleotide
ctcatgcaccttattgataaattatata

SEQ ID NO: 117 - DNA, 18 bp, Artificial Sequence
  Synthetic Oligonucleotide
gccgtggtccatgcatat

SEQ ID NO: 118 - DNA, 26 bp, Artificial Sequence
  Synthetic Oligonucleotide
ccagtaaggtttattaaacaactgag

SEQ ID NO: 119 - DNA, 24 bp, Artificial Sequence
  Synthetic Oligonucleotide
actgtaattcatattcctctacat

SEQ ID NO: 120 - DNA, 28 bp, Artificial Sequence
  Synthetic Oligonucleotide
aatactagtatcaggtaaaccaaattta

SEQ ID NO: 121 - DNA, 23 bp, Artificial Sequence
  Synthetic Oligonucleotide
cataactgtgtctgcttataatc

SEQ ID NO: 122 - DNA, 26 bp, Artificial Sequence
  Synthetic Oligonucleotide
acaaccaataatgcataattgtgttt

SEQ ID NO: 123 - DNA, 21 bp, Artificial Sequence
  Synthetic Oligonucleotide
atccaatggcacatcagattt

SEQ ID NO: 124 - DNA, 26 bp, Artificial Sequence
  Synthetic Oligonucleotide
accaatctattatgtaaataaggcca

SEQ ID NO: 125 - DNA, 21 bp, Artificial Sequence
  Synthetic Oligonucleotide
cctgacacacatttaaacgtt

SEQ ID NO: 126 - DNA, 19 bp, Artificial Sequence
  Synthetic Oligonucleotide
aataatgcgcgggctgcct

SEQ ID NO: 127 - DNA, 20 bp, Artificial Sequence
  Synthetic Oligonucleotide
agtattgccatatccgctgt

SEQ ID NO: 128 - DNA, 27 bp, Artificial Sequence
  Synthetic Oligonucleotide
acataattgtgtttgtttatagtccat

SEQ ID NO: 129 - DNA, 25 bp, Artificial Sequence
  Synthetic Oligonucleotide
tatctaatggaacatcagatttatt

SEQ ID NO: 130 - DNA, 25 bp, Artificial Sequence
  Synthetic Oligonucleotide
gacagtctttcaaagaaacatttcc

SEQ ID NO: 131 - DNA, 23 bp, Artificial Sequence
  Synthetic Oligonucleotide
agtaatacactttccaatcgtat

SEQ ID NO: 132 - DNA, 28 bp, Artificial Sequence
  Synthetic Oligonucleotide
ctccataagcttctttgaatttatataa

SEQ ID NO: 133 - DNA, 20 bp, Artificial Sequence
  Synthetic Oligonucleotide
tcctctgtcacacgttaaac

SEQ ID NO: 134 - DNA, 23 bp, Artificial Sequence
  Synthetic Oligonucleotide
gtacaccttattgtcactaggcc

SEQ ID NO: 135 - DNA, 23 bp, Artificial Sequence
  Synthetic Oligonucleotide
taccctaaacactctatactgat

SEQ ID NO: 136 - DNA, 24 bp, Artificial Sequence
  Synthetic Oligonucleotide
gtctactatgtaaatacggccacc

SEQ ID NO: 137 - DNA, 22 bp, Artificial Sequence
  Synthetic Oligonucleotide
cctgtaacacatttaaacgttg

SEQ ID NO: 138 - DNA, 23 bp, Artificial Sequence
  Synthetic Oligonucleotide
gtccactgaaacattgtccctac

SEQ ID NO: 139 - DNA, 22 bp, Artificial Sequence
  Synthetic Oligonucleotide
agcaccatatcctgtatcaatc

SEQ ID NO: 140 - DNA, 23 bp, Artificial Sequence
  Synthetic Oligonucleotide
gagtggtatcaaccacggtaaca

SEQ ID NO: 141 - DNA, 26 bp, Artificial Sequence
  Synthetic Oligonucleotide
agtatgtatatatgtcattatctctg

SEQ ID NO: 142 - DNA, 27 bp, Artificial Sequence
  Synthetic Oligonucleotide
atatcttaagaattgtactatgggtct

SEQ ID NO: 143 - DNA, 22 bp, Artificial Sequence
  Synthetic Oligonucleotide
ctggtggagtttacatatgaaa

SEQ ID NO: 144 - DNA, 23 bp, Artificial Sequence
  Synthetic Oligonucleotide
acatatgccattattgtggccct

SEQ ID NO: 145 - DNA, 23 bp, Artificial Sequence
  Synthetic Oligonucleotide
atatatgtcataacatcagctgt

SEQ ID NO: 146 - DNA, 24 bp, Artificial Sequence
  Synthetic Oligonucleotide
gaggaagatataccttgctgtcac

SEQ ID NO: 147 - DNA, 23 bp, Artificial Sequence
  Synthetic Oligonucleotide
ctctaaataccctgtactgatag

SEQ ID NO: 148 - DNA, 25 bp, Artificial Sequence
  Synthetic Oligonucleotide
cctgttctataccaatatagtgcag

SEQ ID NO: 149 - DNA, 22 bp, Artificial Sequence
  Synthetic Oligonucleotide
catattcaaatgccatatcgct

SEQ ID NO: 150 - DNA, 27 bp, Artificial Sequence
  Synthetic Oligonucleotide
aatttcactatcatctgttatgtcatg

SEQ ID NO: 151 - DNA, 23 bp, Artificial Sequence
  Synthetic Oligonucleotide
aacatctatattgtagccattgt

SEQ ID NO: 152 - DNA, 23 bp, Artificial Sequence
  Synthetic Oligonucleotide
ctttcgtcccaaaggaaactgat

SEQ ID NO: 153 - DNA, 24 bp, Artificial Sequence
  Synthetic Oligonucleotide
taacacatacaacatatacacaaa

SEQ ID NO: 154 - DNA, 24 bp, Artificial Sequence
  Synthetic Oligonucleotide
aagcatgatttacctgtatttggc

SEQ ID NO: 155 - DNA, 22 bp, Artificial Sequence
  Synthetic Oligonucleotide
cttatgggatttccatctacca

SEQ ID NO: 156 - DNA, 27 bp, Artificial Sequence
  Synthetic Oligonucleotide
attaatatctaatatagcaggtgtggt

SEQ ID NO: 157 - DNA, 25 bp, Artificial Sequence
  Synthetic Oligonucleotide
aacaataaatgtatccataggaatt

SEQ ID NO: 158 - DNA, 24 bp, Artificial Sequence
  Synthetic Oligonucleotide
acagatatgttgtccctaacatcc

SEQ ID NO: 159 - DNA, 19 bp, Artificial Sequence
  Synthetic Oligonucleotide
tgtccaccatatcgccatc

SEQ ID NO: 160 - DNA, 25 bp, Artificial Sequence
  Synthetic Oligonucleotide
aaagctttcaaatgcaggattatca

SEQ ID NO: 161 - DNA, 24 bp, Artificial Sequence
  Synthetic Oligonucleotide
tcctgataataatgtattctagct

SEQ ID NO: 162 - DNA, 26 bp, Artificial Sequence
  Synthetic Oligonucleotide
tcacagtcgtctgttatatcattatc

SEQ ID NO: 163 - DNA, 23 bp, Artificial Sequence
  Synthetic Oligonucleotide
aatcacatctatgttgtatccat

SEQ ID NO: 164 - DNA, 27 bp, Artificial Sequence
  Synthetic Oligonucleotide
gttccaatttgcatcatttaattcata

SEQ ID NO: 165 - DNA, 24 bp, Artificial Sequence
  Synthetic Oligonucleotide
gcactttcataacgtatatatttc

SEQ ID NO: 166 - DNA, 28 bp, Artificial Sequence
  Synthetic Oligonucleotide
tctcttctttagtaatatctatgttgga

SEQ ID NO: 167 - DNA, 20 bp, Artificial Sequence
  Synthetic Oligonucleotide
taaacgcttggctattgctt

SEQ ID NO: 168 - DNA, 28 bp, Artificial Sequence
  Synthetic Oligonucleotide
tccacatttatatcttaataatgctaat

SEQ ID NO: 169 - DNA, 25 bp, Artificial Sequence
  Synthetic Oligonucleotide
tataattgttagtctttgtatccat

SEQ ID NO: 170 - DNA, 28 bp, Artificial Sequence
  Synthetic Oligonucleotide
tattcttaaatctgcaaatacaaagtca

SEQ ID NO: 171 - DNA, 28 bp, Artificial Sequence
  Synthetic Oligonucleotide
gacaaataatacatctaattaatatttc

SEQ ID NO: 172 - DNA, 26 bp, Artificial Sequence
  Synthetic Oligonucleotide
ctgtcaaatggaaatgtatttggaaa

SEQ ID NO: 173 - DNA, 24 bp, Artificial Sequence
  Synthetic Oligonucleotide
tattactgtccagttcataatagt

SEQ ID NO: 174 - DNA, 24 bp, Artificial Sequence
  Synthetic Oligonucleotide
atattcatccgtgcttacaacctt

SEQ ID NO: 175 - DNA, 24 bp, Artificial Sequence
  Synthetic Oligonucleotide
caggtaaatgtattctaaataccc

SEQ ID NO: 176 - DNA, 64 bp, Artificial Sequence
  Synthetic Oligonucleotide
gttggaggctcatcgttcctatattccacaccacttattattacagatgttatgctcgcaggtc

SEQ ID NO: 177 - DNA, 26 bp, Artificial Sequence
  Synthetic Oligonucleotide
gctgtcaccgtccagacgctgttggc

SEQ ID NO: 178 - DNA, 18 bp, Artificial Sequence
  Synthetic Oligonucleotide
tccgtgccttcaagcgcg

SEQ ID NO: 179 - DNA, 28 bp, Artificial Sequence
  Synthetic Oligonucleotide
gactccgcagaatacggcaccgtgcgca

SEQ ID NO: 180 - DNA, 18 bp, Artificial Sequence
  Synthetic Oligonucleotide
gcgtacaggccagtcagc

SEQ ID NO: 181 - DNA, 19 bp, Artificial Sequence
  Synthetic Oligonucleotide
gcagtcggtaacctcgcgc

SEQ ID NO: 182 - DNA, 22 bp, Artificial Sequence
  Synthetic Oligonucleotide
gcgctatctctgctctcactgc

SEQ ID NO: 183 - DNA, 22 bp, Artificial Sequence
  Synthetic Oligonucleotide
gctgtcctggctgcaagcctgg

SEQ ID NO: 184 - DNA, 19 bp, Artificial Sequence
  Synthetic Oligonucleotide
ccgaactgctgatggacgt

SEQ ID NO: 185 - DNA, 28 bp, Artificial Sequence
  Synthetic Oligonucleotide
gacagcagactcaccggctggttccgct

SEQ ID NO: 186 - DNA, 23 bp, Artificial Sequence
  Synthetic Oligonucleotide
gcaagatgctgctggccacactg

SEQ ID NO: 187 - DNA, 22 bp, Artificial Sequence
  Synthetic Oligonucleotide
gacagaacaagttccgctccgg

SEQ ID NO: 188 - DNA, 18 bp, Artificial Sequence
  Synthetic Oligonucleotide
cacggatacgccgcgcat

SEQ ID NO: 189 - DNA, 28 bp, Artificial Sequence
  Synthetic Oligonucleotide
gcaataccaggaaggaagtcttactgct

SEQ ID NO: 190 - DNA, 25 bp, Artificial Sequence
  Synthetic Oligonucleotide
actagtcattggagtacagatgatt

SEQ ID NO: 191 - DNA, 21 bp, Artificial Sequence
  Synthetic Oligonucleotide
gaggaccgaaggagctaaccg

SEQ ID NO: 192 - DNA, 19 bp, Artificial Sequence
  Synthetic Oligonucleotide
cgccgcatacactattctc

SEQ ID NO: 193 - DNA, 27 bp, Artificial Sequence
  Synthetic Oligonucleotide
gctgtaatgcaagtagcgtatgcgctc

SEQ ID NO: 194 - DNA, 27 bp, Artificial Sequence
  Synthetic Oligonucleotide
gaacagcaaggccgccaatgcctgacg

SEQ ID NO: 195 - DNA, 25 bp, Artificial Sequence
  Synthetic Oligonucleotide
gaacgtctggcgctggtcgcctgcc

SEQ ID NO: 196 - DNA, 19 bp, Artificial Sequence
  Synthetic Oligonucleotide
gcacaggtgctgacgtggt

SEQ ID NO: 197 - DNA, 28 bp, Artificial Sequence
  Synthetic Oligonucleotide
cgcatatgctgaatgattatctcgttgc

SEQ ID NO: 198 - DNA, 23 bp, Artificial Sequence
  Synthetic Oligonucleotide
atcttgctcaatgaggttattca

SEQ ID NO: 199 - DNA, 19 bp, Artificial Sequence
  Synthetic Oligonucleotide
gacgacagatgcaggttga

SEQ ID NO: 200 - DNA, 19 bp, Artificial Sequence
  Synthetic Oligonucleotide
cgcatcgccgatgctcatc

SEQ ID NO: 201 - DNA, 28 bp, Artificial Sequence
  Synthetic Oligonucleotide
cgcctgctccagtgcatccagcacgaat

SEQ ID NO: 202 - DNA, 24 bp, Artificial Sequence
  Synthetic Oligonucleotide
atgctctccgccatcgcgttgtca

SEQ ID NO: 203 - DNA, 25 bp, Artificial Sequence
  Synthetic Oligonucleotide
agtgcgttcaccgaatacgtgcgca

SEQ ID NO: 204 - DNA, 20 bp, Artificial Sequence
  Synthetic Oligonucleotide
caggttatgccgctcaattc

SEQ ID NO: 205 - DNA, 27 bp, Artificial Sequence
  Synthetic Oligonucleotide
aatccaggtcctgaccgttctgtccgt

SEQ ID NO: 206 - DNA, 20 bp, Artificial Sequence
  Synthetic Oligonucleotide
acctccgttgagctgatgga

SEQ ID NO: 207 - DNA, 25 bp, Artificial Sequence
  Synthetic Oligonucleotide
gaggtggccaacaccatgtgtgacc

SEQ ID NO: 208 - DNA, 28 bp, Artificial Sequence
  Synthetic Oligonucleotide
gacgccggtatatcggtatcgagctgct

SEQ ID NO: 209 - DNA, 27 bp, Artificial Sequence
  Synthetic Oligonucleotide
cgcatatgctgaatgattatctcgttg

SEQ ID NO: 210 - DNA, 28 bp, Artificial Sequence
  Synthetic Oligonucleotide
acggtgatcttgctcaatgaggttattc

SEQ ID NO: 211 - DNA, 21 bp, Artificial Sequence
  Synthetic Oligonucleotide
gaagtgccggacttctgcaga

SEQ ID NO: 212 - DNA, 21 bp, Artificial Sequence
  Synthetic Oligonucleotide
gcacggcctgatggaggccgc

SEQ ID NO: 213 - DNA, 20 bp, Artificial Sequence
  Synthetic Oligonucleotide
gctaatcgcataacagctac

SEQ ID NO: 214 - DNA, 25 bp, Artificial Sequence
  Synthetic Oligonucleotide
catcacgtaacttattgatgatatt

SEQ ID NO: 215 - DNA, 23 bp, Artificial Sequence
  Synthetic Oligonucleotide
gctgcggtattccacggtcggcc

SEQ ID NO: 216 - DNA, 21 bp, Artificial Sequence
  Synthetic Oligonucleotide
gcaggaacgctgcctgtggtc

SEQ ID NO: 217 - DNA, 27 bp, Artificial Sequence
  Synthetic Oligonucleotide
gaatcaattatcttcttcattattgat

SEQ ID NO: 218 - DNA, 19 bp, Artificial Sequence
  Synthetic Oligonucleotide
ctgcggctcaactcaagca

SEQ ID NO: 219 - DNA, 19 bp, Artificial Sequence
  Synthetic Oligonucleotide
gtcacacgtcacgcagtcc

SEQ ID NO: 220 - DNA, 20 bp, Artificial Sequence
  Synthetic Oligonucleotide
gcattcatggcgctgatggc

SEQ ID NO: 221 - DNA, 27 bp, Artificial Sequence
  Synthetic Oligonucleotide
gtgttactcggtagaatgctcgcaagg

SEQ ID NO: 222 - DNA, 24 bp, Artificial Sequence
  Synthetic Oligonucleotide
actagatgacatatcatgtaagtt

SEQ ID NO: 223 - DNA, 19 bp, Artificial Sequence
  Synthetic Oligonucleotide
cggaactgcctgctcgtat

SEQ ID NO: 224 - DNA, 18 bp, Artificial Sequence
  Synthetic Oligonucleotide
aacgatatagtccgttat

SEQ ID NO: 225 - DNA, 24 bp, Artificial Sequence
  Synthetic Oligonucleotide
gctctccgactcctggtacgtcag

SEQ ID NO: 226 - DNA, 19 bp, Artificial Sequence
  Synthetic Oligonucleotide
gcgcgcattaatgaagcac

SEQ ID NO: 227 - DNA, 28 bp, Artificial Sequence
  Synthetic Oligonucleotide
gatgttgcgattacttcgccaactattg

SEQ ID NO: 228 - DNA, 21 bp, Artificial Sequence
  Synthetic Oligonucleotide
gctgtaattatgacgacgccg

SEQ ID NO: 229 - DNA, 25 bp, Artificial Sequence
  Synthetic Oligonucleotide
ctcattccagaagcaacttcttctt

SEQ ID NO: 230 - DNA, 23 bp, Artificial Sequence
  Synthetic Oligonucleotide
ggatagccatggctacaagaata

SEQ ID NO: 231 - DNA, 26 bp, Artificial Sequence
  Synthetic Oligonucleotide
gcaataccaggaaggaagtcttactg

SEQ ID NO: 232 - DNA, 26 bp, Artificial Sequence
  Synthetic Oligonucleotide
gtcattggagaacagatgattgatgt

SEQ ID NO: 233 - DNA, 24 bp, Artificial Sequence
  Synthetic Oligonucleotide
gtatcgccacaataactgccggaa

SEQ ID NO: 234 - DNA, 19 bp, Artificial Sequence
  Synthetic Oligonucleotide
aacgatatagtccgttatg

SEQ ID NO: 235 - DNA, 23 bp, Artificial Sequence
  Synthetic Oligonucleotide
gctgtggcacaggctgaacgccg

SEQ ID NO: 236 - DNA, 22 bp, Artificial Sequence
  Synthetic Oligonucleotide
ggtgatgtcattctggttaaga

SEQ ID NO: 237 - DNA, 26 bp, Artificial Sequence
  Synthetic Oligonucleotide
acataatctgaatctgagacaacatc

SEQ ID NO: 238 - DNA, 20 bp, Artificial Sequence
  Synthetic Oligonucleotide
acgcactctggccacactgg

SEQ ID NO: 239 - DNA, 20 bp, Artificial Sequence
  Synthetic Oligonucleotide
gtgaagcgcatccggtcacc

SEQ ID NO: 240 - DNA, 22 bp, Artificial Sequence
  Synthetic Oligonucleotide
atggcataggccaggtcaatat

SEQ ID NO: 241 - DNA, 25 bp, Artificial Sequence
  Synthetic Oligonucleotide
ggttctggaccagttgcgtgagcgc

SEQ ID NO: 242 - DNA, 22 bp, Artificial Sequence
  Synthetic Oligonucleotide
cgtaacatcgttgctgctccat

SEQ ID NO: 243 - DNA, 22 bp, Artificial Sequence
  Synthetic Oligonucleotide
cgctggatttcacgccataggc

SEQ ID NO: 244 - DNA, 20 bp, Artificial Sequence
  Synthetic Oligonucleotide
tgtcgctaccgttgatgatt

SEQ ID NO: 245 - DNA, 22 bp, Artificial Sequence
  Synthetic Oligonucleotide
cgtataggtggctaagtgcagc

SEQ ID NO: 246 - DNA, 22 bp, Artificial Sequence
  Synthetic Oligonucleotide
gtaactcattcctgagggtttc

SEQ ID NO: 247 - DNA, 23 bp, Artificial Sequence
  Synthetic Oligonucleotide
gtacatactcgatcgaagcacga

SEQ ID NO: 248 - DNA, 20 bp, Artificial Sequence
  Synthetic Oligonucleotide
ccggaatagcggaagctttc

SEQ ID NO: 249 - DNA, 24 bp, Artificial Sequence
  Synthetic Oligonucleotide
aaggtcgaagcaggtacatactcg

SEQ ID NO: 250 - DNA, 21 bp, Artificial Sequence
  Synthetic Oligonucleotide
agacatgagctcaagtccaat

SEQ ID NO: 251 - DNA, 23 bp, Artificial Sequence
  Synthetic Oligonucleotide
gaagctttcatagcgtcgcctag

SEQ ID NO: 252 - DNA, 23 bp, Artificial Sequence
  Synthetic Oligonucleotide
ttagctagcttgtaagcaaattg

SEQ ID NO: 253 - DNA, 23 bp, Artificial Sequence
  Synthetic Oligonucleotide
gaagctttcatggcatcgcctag

SEQ ID NO: 254 - DNA, 21 bp, Artificial Sequence
  Synthetic Oligonucleotide
agctagcttgtaagcaaactg

SEQ ID NO: 255 - DNA, 22 bp, Artificial Sequence
  Synthetic Oligonucleotide
cgctaccggtagtattgccctt

SEQ ID NO: 256 - DNA, 20 bp, Artificial Sequence
  Synthetic Oligonucleotide
agaatatcccgacggctttc

SEQ ID NO: 257 - DNA, 22 bp, Artificial Sequence
  Synthetic Oligonucleotide
atcgccacgttatcgctgtact

SEQ ID NO: 258 - DNA, 20 bp, Artificial Sequence
  Synthetic Oligonucleotide
tttacccagcgtcagattcc

SEQ ID NO: 259 - DNA, 24 bp, Artificial Sequence
  Synthetic Oligonucleotide
caagtactgttcctgtacgtcagc

SEQ ID NO: 260 - DNA, 21 bp, Artificial Sequence
  Synthetic Oligonucleotide
tcgccagtaactggtctattc

SEQ ID NO: 261 - DNA, 19 bp, Artificial Sequence
  Synthetic Oligonucleotide
caacgtctgcgccatcgcc

SEQ ID NO: 262 - DNA, 20 bp, Artificial Sequence
  Synthetic Oligonucleotide
cgcaatatcattggtggtgc

SEQ ID NO: 263 - DNA, 20 bp, Artificial Sequence
  Synthetic Oligonucleotide
gccgcccgaaggacatcaac

SEQ ID NO: 264 - DNA, 19 bp, Artificial Sequence
  Synthetic Oligonucleotide
cagacgggacgtacacaac

SEQ ID NO: 265 - DNA, 21 bp, Artificial Sequence
  Synthetic Oligonucleotide
cgtgctggctattgccttagg

SEQ ID NO: 266 - DNA, 22 bp, Artificial Sequence
  Synthetic Oligonucleotide
gtaatactcctagcaccaaatc

SEQ ID NO: 267 - DNA, 24 bp, Artificial Sequence
  Synthetic Oligonucleotide
cattaggagttgtcgtatccctca

SEQ ID NO: 268 - DNA, 20 bp, Artificial Sequence
  Synthetic Oligonucleotide
aatactccgagcaccaaatc

SEQ ID NO: 269 - DNA, 21 bp, Artificial Sequence
  Synthetic Oligonucleotide
aaattgcagttcgcgcttagc

SEQ ID NO: 270 - DNA, 21 bp, Artificial Sequence
  Synthetic Oligonucleotide
gttccatagcgttaaggtttc

SEQ ID NO: 271 - DNA, 20 bp, Artificial Sequence
  Synthetic Oligonucleotide
gcgccaaacagaccaatgct

SEQ ID NO: 272 - DNA, 19 bp, Artificial Sequence
  Synthetic Oligonucleotide
gatttcacgccataggctc

SEQ ID NO: 273 - DNA, 22 bp, Artificial Sequence
  Synthetic Oligonucleotide
gtataggtggctaagtgcagca

SEQ ID NO: 274 - DNA, 20 bp, Artificial Sequence
  Synthetic Oligonucleotide
tcgtaactcattcctgaggg

SEQ ID NO: 275 - DNA, 21 bp, Artificial Sequence
  Synthetic Oligonucleotide
gtcatcgcctcttcgtagctc

SEQ ID NO: 276 - DNA, 19 bp, Artificial Sequence
  Synthetic Oligonucleotide
gccatatcgataacgctgg

SEQ ID NO: 277 - DNA, 25 bp, Artificial Sequence
  Synthetic Oligonucleotide
agtatcttacctgaaattccctcac

SEQ ID NO: 278 - DNA, 21 bp, Artificial Sequence
  Synthetic Oligonucleotide
cctctcgtcataagtcgaatg

SEQ ID NO: 279 - DNA, 19 bp, Artificial Sequence
  Synthetic Oligonucleotide
catcacgaagcccgccaca

SEQ ID NO: 280 - DNA, 19 bp, Artificial Sequence
  Synthetic Oligonucleotide
gcccttgagcggaagtatc

SEQ ID NO: 281 - DNA, 21 bp, Artificial Sequence
  Synthetic Oligonucleotide
accaatacgccagtagcgaga

SEQ ID NO: 282 - DNA, 19 bp, Artificial Sequence
  Synthetic Oligonucleotide
gcaacgtagctgccaaatc

SEQ ID NO: 283 - DNA, 24 bp, Artificial Sequence
  Synthetic Oligonucleotide
caatcagtgtgtttgatttgcacc

SEQ ID NO: 284 - DNA, 19 bp, Artificial Sequence
  Synthetic Oligonucleotide
tacccggaatagcctgctc

SEQ ID NO: 285 - DNA, 20 bp, Artificial Sequence
  Synthetic Oligonucleotide
cggataacgccacgggatga

SEQ ID NO: 286 - DNA, 20 bp, Artificial Sequence
  Synthetic Oligonucleotide
accgggtcaaagaattcctc

SEQ ID NO: 287 - DNA, 18 bp, Artificial Sequence
  Synthetic Oligonucleotide
gcggcgtggtggtgtctc

SEQ ID NO: 288 - DNA, 18 bp, Artificial Sequence
  Synthetic Oligonucleotide
cgctgccggtcttatcac

SEQ ID NO: 289 - DNA, 18 bp, Artificial Sequence
  Synthetic Oligonucleotide
gccacgtcaccagctgcg

SEQ ID NO: 290 - DNA, 19 bp, Artificial Sequence
  Synthetic Oligonucleotide
cggctgggtgaagtaagtc

SEQ ID NO: 291 - DNA, 19 bp, Artificial Sequence
  Synthetic Oligonucleotide
gctcgtagcgtcgcgtctc

SEQ ID NO: 292 - DNA, 18 bp, Artificial Sequence
  Synthetic Oligonucleotide
ttgaccgacagaggcaac

SEQ ID NO: 293 - DNA, 21 bp, Artificial Sequence
  Synthetic Oligonucleotide
cagcaggtccgccaatttctc

SEQ ID NO: 294 - DNA, 17 bp, Artificial Sequence
  Synthetic Oligonucleotide
agtggacgtcagtgcgc

SEQ ID NO: 295 - DNA, 19 bp, Artificial Sequence
  Synthetic Oligonucleotide
cgtagtgtcgcgtctcccg

SEQ ID NO: 296 - DNA, 23 bp, Artificial Sequence
  Synthetic Oligonucleotide
caggatgagttgtgtaataactt

SEQ ID NO: 297 - DNA, 23 bp, Artificial Sequence
  Synthetic Oligonucleotide
ccatagaggactttagccacagt

SEQ ID NO: 298 - DNA, 19 bp, Artificial Sequence
  Synthetic Oligonucleotide
tacaccgctacagcgtaat

SEQ ID NO: 299 - DNA, 21 bp, Artificial Sequence
  Synthetic Oligonucleotide
catatgcagagtgagcggtcc

SEQ ID NO: 300 - DNA, 21 bp, Artificial Sequence
  Synthetic Oligonucleotide
tcaattctttcaaagaccagc

SEQ ID NO: 301 - DNA, 26 bp, Artificial Sequence
  Synthetic Oligonucleotide
ccattaacttcttcaaacgatgtatg

SEQ ID NO: 302 - DNA, 17 bp, Artificial Sequence
  Synthetic Oligonucleotide
acccgtgctgtcgctat

SEQ ID NO: 303 - DNA, 22 bp, Artificial Sequence
  Synthetic Oligonucleotide
gtgctgtcgctatggaaatgtg

SEQ ID NO: 304 - DNA, 22 bp, Artificial Sequence
  Synthetic Oligonucleotide
aaccaaaccactaggttatctt

SEQ ID NO: 305 - DNA, 23 bp, Artificial Sequence
  Synthetic Oligonucleotide
gtcagtgtttacaagaaccacca

SEQ ID NO: 306 - DNA, 21 bp, Artificial Sequence
  Synthetic Oligonucleotide
atgcatacgtgggaatagatt

SEQ ID NO: 307 - DNA, 18 bp, Artificial Sequence
  Synthetic Oligonucleotide
cggaagtatccgcgcgcc

SEQ ID NO: 308 - DNA, 18 bp, Artificial Sequence
  Synthetic Oligonucleotide
ttcgatcacggcacgatc

SEQ ID NO: 309 - DNA, 21 bp, Artificial Sequence
  Synthetic Oligonucleotide
cgaaccagcttggttcccaag

SEQ ID NO: 310 - DNA, 17 bp, Artificial Sequence
  Synthetic Oligonucleotide
tcactgcgtgttcgctc

SEQ ID NO: 311 - DNA, 23 bp, Artificial Sequence
  Synthetic Oligonucleotide
gatgctgtactttgtgatgccta

SEQ ID NO: 312 - DNA, 19 bp, Artificial Sequence
  Synthetic Oligonucleotide
cgcttggcaagtactgttc

SEQ ID NO: 313 - DNA, 22 bp, Artificial Sequence
  Synthetic Oligonucleotide
gcaagaaagcccttgaatgagc

SEQ ID NO: 314 - DNA, 20 bp, Artificial Sequence
  Synthetic Oligonucleotide
gcgttatcactgtattgcac

SEQ ID NO: 315 - DNA, 21 bp, Artificial Sequence
  Synthetic Oligonucleotide
aatcaacaaactgctgccgct

SEQ ID NO: 316 - DNA, 20 bp, Artificial Sequence
  Synthetic Oligonucleotide
gctgtacttgtcatccttgt

SEQ ID NO: 317 - DNA, 18 bp, Artificial Sequence
  Synthetic Oligonucleotide
ccagtctgccggcaccgc

SEQ ID NO: 318 - DNA, 17 bp, Artificial Sequence
  Synthetic Oligonucleotide
tcgagcgcgagtctagc

SEQ ID NO: 319 - DNA, 19 bp, Artificial Sequence
  Synthetic Oligonucleotide
ccgactgcccagtctgccg

SEQ ID NO: 320 - DNA, 17 bp, Artificial Sequence
  Synthetic Oligonucleotide
cgagcgcgagtctagcc

SEQ ID NO: 321 - DNA, 28 bp, Artificial Sequence
  Synthetic Oligonucleotide
gtaaatagatgatcttaatttggttcac

SEQ ID NO: 322 - DNA, 17 bp, Artificial Sequence
  Synthetic Oligonucleotide
ttgctggccaatcgtcg

SEQ ID NO: 323 - DNA, 20 bp, Artificial Sequence
  Synthetic Oligonucleotide
cacagcctgactttcgccgc

SEQ ID NO: 324 - DNA, 20 bp, Artificial Sequence
  Synthetic Oligonucleotide
caagcaggagatcaacctgc

SEQ ID NO: 325 - DNA, 19 bp, Artificial Sequence
  Synthetic Oligonucleotide
ggtggtcgataccgcctgg

SEQ ID NO: 326 - DNA, 17 bp, Artificial Sequence
  Synthetic Oligonucleotide
gtgaaatccgcccgacg

SEQ ID NO: 327 - DNA, 22 bp, Artificial Sequence
  Synthetic Oligonucleotide
catgtcgagataggaagtgtgc

SEQ ID NO: 328 - DNA, 17 bp, Artificial Sequence
  Synthetic Oligonucleotide
tgatgcgcgtgagtcac

SEQ ID NO: 329 - DNA, 20 bp, Artificial Sequence
  Synthetic Oligonucleotide
caatctgccatcgcgcgatt

SEQ ID NO: 330 - DNA, 18 bp, Artificial Sequence
  Synthetic Oligonucleotide
cggcaatctcggtgatgc

SEQ ID NO: 331 - DNA, 22 bp, Artificial Sequence
  Synthetic Oligonucleotide
cgaagcaggtacatactcggtc

SEQ ID NO: 332 - DNA, 23 bp, Artificial Sequence
  Synthetic Oligonucleotide
acgagctaaatcttgataaactt

SEQ ID NO: 333 - DNA, 23 bp, Artificial Sequence
  Synthetic Oligonucleotide
tagaatagcggaagctttcatgg

SEQ ID NO: 334 - DNA, 21 bp, Artificial Sequence
  Synthetic Oligonucleotide
agctagcttgtaagcaaactg

SEQ ID NO: 335 - DNA, 23 bp, Artificial Sequence
  Synthetic Oligonucleotide
caagtccaatacgacgagctaaa

SEQ ID NO: 336 - DNA, 21 bp, Artificial Sequence
  Synthetic Oligonucleotide
gaatagcatggattgcacttc

SEQ ID NO: 337 - DNA, 22 bp, Artificial Sequence
  Synthetic Oligonucleotide
ggtacatactcggtcgaagcac

SEQ ID NO: 338 - DNA, 23 bp, Artificial Sequence
  Synthetic Oligonucleotide
aatcttgataaactgaaatagcg

SEQ ID NO: 339 - DNA, 22 bp, Artificial Sequence
  Synthetic Oligonucleotide
ggtacatactcggtcgatgcac

SEQ ID NO: 340 - DNA, 21 bp, Artificial Sequence
  Synthetic Oligonucleotide
tcttgataaaccggaatagcg

SEQ ID NO: 341 - DNA, 25 bp, Artificial Sequence
  Synthetic Oligonucleotide
gtaattgaactagctaatgccgtac

SEQ ID NO: 342 - DNA, 21 bp, Artificial Sequence
  Synthetic Oligonucleotide
ttatgacaccagtttctaggc

SEQ ID NO: 343 - DNA, 23 bp, Artificial Sequence
  Synthetic Oligonucleotide
caagtactgttcctgtacgtcag

SEQ ID NO: 344 - DNA, 19 bp, Artificial Sequence
  Synthetic Oligonucleotide
gcccagttgtgatgcattc

SEQ ID NO: 345 - DNA, 23 bp, Artificial Sequence
  Synthetic Oligonucleotide
tctctttcccattgtttcatggc

SEQ ID NO: 346 - DNA, 20 bp, Artificial Sequence
  Synthetic Oligonucleotide
tgcggaaattctaagctgac

SEQ ID NO: 347 - DNA, 26 bp, Artificial Sequence
  Synthetic Oligonucleotide
gtaggttatgcagttattaggttcag

SEQ ID NO: 348 - DNA, 18 bp, Artificial Sequence
  Synthetic Oligonucleotide
gactcagccgagtcaagc

SEQ ID NO: 349 - DNA, 23 bp, Artificial Sequence
  Synthetic Oligonucleotide
gcagtaccaacatagctaaatgc

SEQ ID NO: 350 - DNA, 21 bp, Artificial Sequence
  Synthetic Oligonucleotide
aaataacaaatcacaggccac

SEQ ID NO: 351 - DNA, 21 bp, Artificial Sequence
  Synthetic Oligonucleotide
ggtcctgtggtggtttccacc

SEQ ID NO: 352 - DNA, 20 bp, Artificial Sequence
  Synthetic Oligonucleotide
cgcgataatggcttcattgg

SEQ ID NO: 353 - DNA, 20 bp, Artificial Sequence
  Synthetic Oligonucleotide
taaccgctgtggtcctgtgg

SEQ ID NO: 354 - DNA, 21 bp, Artificial Sequence
  Synthetic Oligonucleotide
tgcgcaataatagcttcattg

SEQ ID NO: 355 - DNA, 21 bp, Artificial Sequence
  Synthetic Oligonucleotide
ggaagcgttgcttgccatagt

SEQ ID NO: 356 - DNA, 20 bp, Artificial Sequence
  Synthetic Oligonucleotide
aaccgaagcaccatgtaatt

SEQ ID NO: 357 - DNA, 19 bp, Artificial Sequence
  Synthetic Oligonucleotide
gttcggtgcaaagacgccg

SEQ ID NO: 358 - DNA, 23 bp, Artificial Sequence
  Synthetic Oligonucleotide
tcgcagacttcaatatcaatatt

SEQ ID NO: 359 - DNA, 21 bp, Artificial Sequence
  Synthetic Oligonucleotide
cacctgatgcagaaccagcat

SEQ ID NO: 360 - DNA, 19 bp, Artificial Sequence
  Synthetic Oligonucleotide
aggccacgttatcactgtg

SEQ ID NO: 361 - DNA, 18 bp, Artificial Sequence
  Synthetic Oligonucleotide
cagctgccgttgcgaacg

SEQ ID NO: 362 - DNA, 21 bp, Artificial Sequence
  Synthetic Oligonucleotide
cgcagataaatcaccacaatc

SEQ ID NO: 363 - DNA, 19 bp, Artificial Sequence
  Synthetic Oligonucleotide
gctcagacgctggctggtc

SEQ ID NO: 364 - DNA, 19 bp, Artificial Sequence
  Synthetic Oligonucleotide
ccgcagataaatcaccacg

SEQ ID NO: 365 - DNA, 20 bp, Artificial Sequence
  Synthetic Oligonucleotide
gccagtagcagattggcggc

SEQ ID NO: 366 - DNA, 17 bp, Artificial Sequence
  Synthetic Oligonucleotide
gaacgggcgctcagacg

SEQ ID NO: 367 - DNA, 19 bp, Artificial Sequence
  Synthetic Oligonucleotide
ccactgcagcagatgccgt

SEQ ID NO: 368 - DNA, 21 bp, Artificial Sequence
  Synthetic Oligonucleotide
gtatcccgcagataaatcacc

SEQ ID NO: 369 - DNA, 22 bp, Artificial Sequence
  Synthetic Oligonucleotide
ttaatttgcttaagcggctgcg

SEQ ID NO: 370 - DNA, 17 bp, Artificial Sequence
  Synthetic Oligonucleotide
ccagctgttcgtcaccg

SEQ ID NO: 371 - DNA, 19 bp, Artificial Sequence
  Synthetic Oligonucleotide
gggaaagcgttcatcggcg

SEQ ID NO: 372 - DNA, 18 bp, Artificial Sequence
  Synthetic Oligonucleotide
tcgctcatggtaatggcg

SEQ ID NO: 373 - DNA, 18 bp, Artificial Sequence
  Synthetic Oligonucleotide
gcgaacgggcgctcagac

SEQ ID NO: 374 - DNA, 20 bp, Artificial Sequence
  Synthetic Oligonucleotide
ataaatcaccacaatgcgct

SEQ ID NO: 375 - DNA, 22 bp, Artificial Sequence
  Synthetic Oligonucleotide
tcttatcggcgataaaccagcc

SEQ ID NO: 376 - DNA, 17 bp, Artificial Sequence
  Synthetic Oligonucleotide
cgttgccagtgctcgat

SEQ ID NO: 377 - DNA, 24 bp, Artificial Sequence
  Synthetic Oligonucleotide
cagtccctcgatattcagatcaga

SEQ ID NO: 378 - DNA, 20 bp, Artificial Sequence
  Synthetic Oligonucleotide
ttaacaatttcgcaaccgtc

SEQ ID NO: 379 - DNA, 21 bp, Artificial Sequence
  Synthetic Oligonucleotide
cagctgcggtaaagctcatca

SEQ ID NO: 380 - DNA, 23 bp, Artificial Sequence
  Synthetic Oligonucleotide
catagttaagccagtatacactc

SEQ ID NO: 381 - DNA, 23 bp, Artificial Sequence
  Synthetic Oligonucleotide
gtcggaaagttgaccagacatta

SEQ ID NO: 382 - DNA, 25 bp, Artificial Sequence
  Synthetic Oligonucleotide
atactaggagaagttaataaatacg

SEQ ID NO: 383 - DNA, 27 bp, Artificial Sequence
  Synthetic Oligonucleotide
cattctctcgctttaatttattaacct

SEQ ID NO: 384 - DNA, 21 bp, Artificial Sequence
  Synthetic Oligonucleotide
atcgaccttctggacattatc

SEQ ID NO: 385 - DNA, 25 bp, Artificial Sequence
  Synthetic Oligonucleotide
gtaacaactttcatgctctcctaaa

SEQ ID NO: 386 - DNA, 20 bp, Artificial Sequence
  Synthetic Oligonucleotide
cggtaactgatgccgtattt

SEQ ID NO: 387 - DNA, 24 bp, Artificial Sequence
  Synthetic Oligonucleotide
gtgaagtgaatggtcagtatgttg

SEQ ID NO: 388 - DNA, 18 bp, Artificial Sequence
  Synthetic Oligonucleotide
agtgcgcaggagattagc

SEQ ID NO: 389 - DNA, 22 bp, Artificial Sequence
  Synthetic Oligonucleotide
cctgtcctacgagttgcatgat

SEQ ID NO: 390 - DNA, 19 bp, Artificial Sequence
  Synthetic Oligonucleotide
ataatggcctgcttctcgc

SEQ ID NO: 391 - DNA, 23 bp, Artificial Sequence
  Synthetic Oligonucleotide
cgtttccagactttacgaaacac

SEQ ID NO: 392 - DNA, 20 bp, Artificial Sequence
  Synthetic Oligonucleotide
acgttgtgagggtaaacaac

SEQ ID NO: 393 - DNA, 23 bp, Artificial Sequence
  Synthetic Oligonucleotide
cgttgcttacgcaaccaaatatc

SEQ ID NO: 394 - DNA, 21 bp, Artificial Sequence
  Synthetic Oligonucleotide
tgatcttgctcaatgaggtta

SEQ ID NO: 395 - DNA, 26 bp, Artificial Sequence
  Synthetic Oligonucleotide
catcatgttcatatttatcagagctc

SEQ ID NO: 396 - DNA, 24 bp, Artificial Sequence
  Synthetic Oligonucleotide
tagatttcataaagtctaacacac

SEQ ID NO: 397 - DNA, 21 bp, Artificial Sequence
  Synthetic Oligonucleotide
gtttccacatggtgaacggtg

SEQ ID NO: 398 - DNA, 21 bp, Artificial Sequence
  Synthetic Oligonucleotide
aaacctgtcactctgaatgtt

SEQ ID NO: 399 - DNA, 29 bp, Artificial Sequence
  Synthetic Oligonucleotide
caaatactaaattatacagtatcagagag

SEQ ID NO: 400 - DNA, 22 bp, Artificial Sequence
  Synthetic Oligonucleotide
atgcaaagcgttatgaaatttc

SEQ ID NO: 401 - DNA, 32 bp, Artificial Sequence
  Synthetic Oligonucleotide
gttcttattattataagtatctattaacagtt

SEQ ID NO: 402 - DNA, 19 bp, Artificial Sequence
  Synthetic Oligonucleotide
cattagtggctgctgcaat

SEQ ID NO: 403 - DNA, 23 bp, Artificial Sequence
  Synthetic Oligonucleotide
catcgggaaatggaagtcgttat

SEQ ID NO: 404 - DNA, 21 bp, Artificial Sequence
  Synthetic Oligonucleotide
gttcaatcgtcaaagttgttc

SEQ ID NO: 405 - DNA, 21 bp, Artificial Sequence
  Synthetic Oligonucleotide
cgtggtttgtgctgagcaaag

SEQ ID NO: 406 - DNA, 23 bp, Artificial Sequence
  Synthetic Oligonucleotide
caaagttaagttgtcagtttgag

SEQ ID NO: 407 - DNA, 19 bp, Artificial Sequence
  Synthetic Oligonucleotide
gccgcccgaaggacatcaa

SEQ ID NO: 408 - DNA, 18 bp, Artificial Sequence
  Synthetic Oligonucleotide
agacgggacgtacacaac

SEQ ID NO: 409 - DNA, 21 bp, Artificial Sequence
  Synthetic Oligonucleotide
gcaactcatcaccatcacgga

SEQ ID NO: 410 - DNA, 18 bp, Artificial Sequence
  Synthetic Oligonucleotide
tgatgcgtacgttgccac

SEQ ID NO: 411 - DNA, 20 bp, Artificial Sequence
  Synthetic Oligonucleotide
gcgacagccatgacagacgc

SEQ ID NO: 412 - DNA, 20 bp, Artificial Sequence
  Synthetic Oligonucleotide
ggacaatgagaccattggac

SEQ ID NO: 413 - DNA, 21 bp, Artificial Sequence
  Synthetic Oligonucleotide
aaacgactgcgttgcgatatg

SEQ ID NO: 414 - DNA, 19 bp, Artificial Sequence
  Synthetic Oligonucleotide
ttccgaaggacatcaacgc

SEQ ID NO: 415 - DNA, 20 bp, Artificial Sequence
  Synthetic Oligonucleotide
atgcgaccaaacgccatcgc

SEQ ID NO: 416 - DNA, 19 bp, Artificial Sequence
  Synthetic Oligonucleotide
atcgtcatggaagtgcgta

SEQ ID NO: 417 - DNA, 22 bp, Artificial Sequence
  Synthetic Oligonucleotide
gtcatgaaagtgcgtggagact

SEQ ID NO: 418 - DNA, 19 bp, Artificial Sequence
  Synthetic Oligonucleotide
accgggatagaagagctct

SEQ ID NO: 419 - DNA, 22 bp, Artificial Sequence
  Synthetic Oligonucleotide
gaacaggcttatgtcaactggg

SEQ ID NO: 420 - DNA, 21 bp, Artificial Sequence
  Synthetic Oligonucleotide
cataacatcaaacatcgaccc

SEQ ID NO: 421 - DNA, 22 bp, Artificial Sequence
  Synthetic Oligonucleotide
acgaaccgaacaggcttatgtc

SEQ ID NO: 422 - DNA, 17 bp, Artificial Sequence
  Synthetic Oligonucleotide
taacgcgcttgctgctt

SEQ ID NO: 423 - DNA, 21 bp, Artificial Sequence
  Synthetic Oligonucleotide
gctgtaattatgacgacgccg

SEQ ID NO: 424 - DNA, 20 bp, Artificial Sequence
  Synthetic Oligonucleotide
ctcggtgagattcagaatgc

SEQ ID NO: 425 - DNA, 23 bp, Artificial Sequence
  Synthetic Oligonucleotide
catcatagacgcggtcaaataga

SEQ ID NO: 426 - DNA, 19 bp, Artificial Sequence
  Synthetic Oligonucleotide
actcatcaccatcacggac

SEQ ID NO: 427 - DNA, 23 bp, Artificial Sequence
  Synthetic Oligonucleotide
gtgtatgtcagcgatttgtccat

SEQ ID NO: 428 - DNA, 21 bp, Artificial Sequence
  Synthetic Oligonucleotide
tgtcatattgtcttgccgatt

SEQ ID NO: 429 - DNA, 21 bp, Artificial Sequence
  Synthetic Oligonucleotide
gtccacctcgccaacaatcaa

SEQ ID NO: 430 - DNA, 21 bp, Artificial Sequence
  Synthetic Oligonucleotide
atatcaacacgggaaagacct

SEQ ID NO: 431 - DNA, 21 bp, Artificial Sequence
  Synthetic Oligonucleotide
gcgtgattatcacgttcggca

SEQ ID NO: 432 - DNA, 20 bp, Artificial Sequence
  Synthetic Oligonucleotide
cttgcagatttaaccgacac

SEQ ID NO: 433 - DNA, 23 bp, Artificial Sequence
  Synthetic Oligonucleotide
ggctcgacttcctgatgaatacg

SEQ ID NO: 434 - DNA, 19 bp, Artificial Sequence
  Synthetic Oligonucleotide
tgaaaccgggcagagtatt

SEQ ID NO: 435 - DNA, 24 bp, Artificial Sequence
  Synthetic Oligonucleotide
caacgatgtatgtcaacgatttgt

SEQ ID NO: 436 - DNA, 20 bp, Artificial Sequence
  Synthetic Oligonucleotide
attgcgtagtccaattcgtc

SEQ ID NO: 437 - DNA, 20 bp, Artificial Sequence
  Synthetic Oligonucleotide
caggctgtttcgggctgtga

SEQ ID NO: 438 - DNA, 24 bp, Artificial Sequence
  Synthetic Oligonucleotide
gggttattaataaagatgataggc

SEQ ID NO: 439 - DNA, 22 bp, Artificial Sequence
  Synthetic Oligonucleotide
ggctcggcttcctgatgaatac

SEQ ID NO: 440 - DNA, 21 bp, Artificial Sequence
  Synthetic Oligonucleotide
aggcatggtattgacttcatt

SEQ ID NO: 441 - DNA, 23 bp, Artificial Sequence
  Synthetic Oligonucleotide
taattcaagtgcaactctcgcaa

SEQ ID NO: 442 - DNA, 25 bp, Artificial Sequence
  Synthetic Oligonucleotide
tttattctctaatgcgctatatatt

SEQ ID NO: 443 - DNA, 24 bp, Artificial Sequence
  Synthetic Oligonucleotide
ggatagttacgactttctgcttca

SEQ ID NO: 444 - DNA, 22 bp, Artificial Sequence
  Synthetic Oligonucleotide
tgtattgctattatcgtcaacg

SEQ ID NO: 445 - DNA, 24 bp, Artificial Sequence
  Synthetic Oligonucleotide
cagtatttcaccttgtccgtaacc

SEQ ID NO: 446 - DNA, 20 bp, Artificial Sequence
  Synthetic Oligonucleotide
gtttacgacttgttgcatgc

SEQ ID NO: 447 - DNA, 27 bp, Artificial Sequence
  Synthetic Oligonucleotide
aatgtttatatctttaacgcctaaact

SEQ ID NO: 448 - DNA, 20 bp, Artificial Sequence
  Synthetic Oligonucleotide
atgctttggtctttctgcat

SEQ ID NO: 449 - DNA, 19 bp, Artificial Sequence
  Synthetic Oligonucleotide
ctggcccttgaggtcgcgg

SEQ ID NO: 450 - DNA, 18 bp, Artificial Sequence
  Synthetic Oligonucleotide
cggtcttcacctcgacac

SEQ ID NO: 451 - DNA, 20 bp, Artificial Sequence
  Synthetic Oligonucleotide
gacgtagatcgggtcgagct

SEQ ID NO: 452 - DNA, 19 bp, Artificial Sequence
  Synthetic Oligonucleotide
acggaaacctcggagaatt

SEQ ID NO: 453 - DNA, 20 bp, Artificial Sequence
  Synthetic Oligonucleotide
ggcgtactgctgcttgctca

SEQ ID NO: 454 - DNA, 18 bp, Artificial Sequence
  Synthetic Oligonucleotide
tgacgtcgacgtagatcg

SEQ ID NO: 455 - DNA, 20 bp, Artificial Sequence
  Synthetic Oligonucleotide
cctgttcctgggtcgaagcc

SEQ ID NO: 456 - DNA, 16 bp, Artificial Sequence
  Synthetic Oligonucleotide
cttcggtcaccgcgga

SEQ ID NO: 457 - DNA, 27 bp, Artificial Sequence
  Synthetic Oligonucleotide
gtcaggctaaatatagctatcttatcg

SEQ ID NO: 458 - DNA, 24 bp, Artificial Sequence
  Synthetic Oligonucleotide
tcagttactgctatagaaattgat

SEQ ID NO: 459 - DNA, 23 bp, Artificial Sequence
  Synthetic Oligonucleotide
catcctaagccaagtgtagactc

SEQ ID NO: 460 - DNA, 27 bp, Artificial Sequence
  Synthetic Oligonucleotide
aagatatatggtaatattccttataac

SEQ ID NO: 461 - DNA, 25 bp, Artificial Sequence
  Synthetic Oligonucleotide
gtttataagtgggtaaaccgtgaat

SEQ ID NO: 462 - DNA, 20 bp, Artificial Sequence
  Synthetic Oligonucleotide
gaaacgagctttaggtttgc

SEQ ID NO: 463 - DNA, 27 bp, Artificial Sequence
  Synthetic Oligonucleotide
gcagcacttgaccgccatgagtgacca

SEQ ID NO: 464 - DNA, 23 bp, Artificial Sequence
  Synthetic Oligonucleotide
catcgcaccaacaacaataatcg

SEQ ID NO: 465 - DNA, 26 bp, Artificial Sequence
  Synthetic Oligonucleotide
gtgatcactgatgcaccagatgaagt

SEQ ID NO: 466 - DNA, 24 bp, Artificial Sequence
  Synthetic Oligonucleotide
atcttgatattcaagtctatgacg

SEQ ID NO: 467 - DNA, 28 bp, Artificial Sequence
  Synthetic Oligonucleotide
gatattattgatcatggtgccaagccaa

SEQ ID NO: 468 - DNA, 21 bp, Artificial Sequence
  Synthetic Oligonucleotide
caatatgaagctgacgacgcg

SEQ ID NO: 469 - DNA, 28 bp, Artificial Sequence
  Synthetic Oligonucleotide
gctgagcgtgaaggttcatggattatta

SEQ ID NO: 470 - DNA, 20 bp, Artificial Sequence
  Synthetic Oligonucleotide
ggtaaggcttacggtctcat

SEQ ID NO: 471 - DNA, 28 bp, Artificial Sequence
  Synthetic Oligonucleotide
gcatcttgtgcagcctgaatagcagcgt

SEQ ID NO: 472 - DNA, 26 bp, Artificial Sequence
  Synthetic Oligonucleotide
accacgttgaatatcaccttcggcat

SEQ ID NO: 473 - DNA, 28 bp, Artificial Sequence
  Synthetic Oligonucleotide
aagtccataattgcttgagtgtagtcat

SEQ ID NO: 474 - DNA, 26 bp, Artificial Sequence
  Synthetic Oligonucleotide
atcttcgcactgaataataagaacat

SEQ ID NO: 475 - DNA, 28 bp, Artificial Sequence
  Synthetic Oligonucleotide
gcttgctggttctgcacgtagcttactg

SEQ ID NO: 476 - DNA, 21 bp, Artificial Sequence
  Synthetic Oligonucleotide
aagatgaacaggctactgcaa

SEQ ID NO: 477 - DNA, 28 bp, Artificial Sequence
  Synthetic Oligonucleotide
gcagcgctgtgcaagttcaatgtattct

SEQ ID NO: 478 - DNA, 24 bp, Artificial Sequence
  Synthetic Oligonucleotide
ctcgtgcgagtattccttaagtgt

SEQ ID NO: 479 - DNA, 28 bp, Artificial Sequence
  Synthetic Oligonucleotide
gtataacactcggccagcgccaaggttc

SEQ ID NO: 480 - DNA, 24 bp, Artificial Sequence
  Synthetic Oligonucleotide
gttcacacatcgccacaatatgat

SEQ ID NO: 481 - DNA, 22 bp, Artificial Sequence
  Synthetic Oligonucleotide
accatgcagatacaatgaacca

SEQ ID NO: 482 - DNA, 23 bp, Artificial Sequence
  Synthetic Oligonucleotide
ggatgataagacacatccaattc

SEQ ID NO: 483 - DNA, 24 bp, Artificial Sequence
  Synthetic Oligonucleotide
catcaacagcttcttgaagcattc

SEQ ID NO: 484 - DNA, 24 bp, Artificial Sequence
  Synthetic Oligonucleotide
gtccaacaactataacagaacgtc

SEQ ID NO: 485 - DNA, 26 bp, Artificial Sequence
  Synthetic Oligonucleotide
aacatatcacctgatattctagtatc

SEQ ID NO: 486 - DNA, 27 bp, Artificial Sequence
  Synthetic Oligonucleotide
attccattatattcaacaggattgtga

SEQ ID NO: 487 - DNA, 20 bp, Artificial Sequence
  Synthetic Oligonucleotide
gctgttgcttgcggatactg

SEQ ID NO: 488 - DNA, 21 bp, Artificial Sequence
  Synthetic Oligonucleotide
cgtatatgtagctcaagttgc

SEQ ID NO: 489 - DNA, 27 bp, Artificial Sequence
  Synthetic Oligonucleotide
aagagctaatgcagctattgcacttat

SEQ ID NO: 490 - DNA, 24 bp, Artificial Sequence
  Synthetic Oligonucleotide
catacacttcagctataagaccat

SEQ ID NO: 491 - DNA, 24 bp, Artificial Sequence
  Synthetic Oligonucleotide
aacaagagcagaagttacagacgt

SEQ ID NO: 492 - DNA, 22 bp, Artificial Sequence
  Synthetic Oligonucleotide
gtataatggtggctagaggtga

SEQ ID NO: 493 - DNA, 25 bp, Artificial Sequence
  Synthetic Oligonucleotide
actcgtgaagaccatgcagatacaa

SEQ ID NO: 494 - DNA, 21 bp, Artificial Sequence
  Synthetic Oligonucleotide
aatacttacaatgcctgagga

SEQ ID NO: 495 - DNA, 21 bp, Artificial Sequence
  Synthetic Oligonucleotide
accatgcagatacaatgaacc

SEQ ID NO: 496 - DNA, 22 bp, Artificial Sequence
  Synthetic Oligonucleotide
cctgaggatgataagacacatc

SEQ ID NO: 497 - DNA, 27 bp, Artificial Sequence
  Synthetic Oligonucleotide
gcatctgctgcttctattgctcctact

SEQ ID NO: 498 - DNA, 25 bp, Artificial Sequence
  Synthetic Oligonucleotide
acatgaactgatattagttctccaa

SEQ ID NO: 499 - DNA, 23 bp, Artificial Sequence
  Synthetic Oligonucleotide
gcacaagctggagataacatcgg

SEQ ID NO: 500 - DNA, 24 bp, Artificial Sequence
  Synthetic Oligonucleotide
gtagaggacgtattcacaatcact

SEQ ID NO: 501 - DNA, 25 bp, Artificial Sequence
  Synthetic Oligonucleotide
ctctatcagcttctactgcttcttc

SEQ ID NO: 502 - DNA, 25 bp, Artificial Sequence
  Synthetic Oligonucleotide
ccatctcatccacagttaatatatc

SEQ ID NO: 503 - DNA, 28 bp, Artificial Sequence
  Synthetic Oligonucleotide
agatgagattcatactatcgttggagct

SEQ ID NO: 504 - DNA, 24 bp, Artificial Sequence
  Synthetic Oligonucleotide
agcagagagaatagtaagaggaga

SEQ ID NO: 505 - DNA, 23 bp, Artificial Sequence
  Synthetic Oligonucleotide
catcaacagcttcttgaagcatt

SEQ ID NO: 506 - DNA, 22 bp, Artificial Sequence
  Synthetic Oligonucleotide
gtccaacaactataacagaacg

SEQ ID NO: 507 - DNA, 27 bp, Artificial Sequence
  Synthetic Oligonucleotide
gtcagcaatacgccaccaagctcctat

SEQ ID NO: 508 - DNA, 20 bp, Artificial Sequence
  Synthetic Oligonucleotide
gtggtggatatcctgttacc

SEQ ID NO: 509 - DNA, 27 bp, Artificial Sequence
  Synthetic Oligonucleotide
gcgcaatagagttgtataagagtgctg

SEQ ID NO: 510 - DNA, 28 bp, Artificial Sequence
  Synthetic Oligonucleotide
agcattaattatagattataatgtataa

SEQ ID NO: 511 - DNA, 23 bp, Artificial Sequence
  Synthetic Oligonucleotide
ggcataataggatggatagatga

SEQ ID NO: 512 - DNA, 23 bp, Artificial Sequence
  Synthetic Oligonucleotide
actaatccaacttctactgctat

SEQ ID NO: 513 - DNA, 26 bp, Artificial Sequence
  Synthetic Oligonucleotide
gtacattcacatatagaccatcttaa

SEQ ID NO: 514 - DNA, 25 bp, Artificial Sequence
  Synthetic Oligonucleotide
acataggtgcaggtagaatagtata

SEQ ID NO: 515 - DNA, 24 bp, Artificial Sequence
  Synthetic Oligonucleotide
ccataccagtatcttggcatattg

SEQ ID NO: 516 - DNA, 25 bp, Artificial Sequence
  Synthetic Oligonucleotide
ataatgaataacagcaggtgtatta

SEQ ID NO: 517 - DNA, 24 bp, Artificial Sequence
  Synthetic Oligonucleotide
agatgaagcacaagctggagataa

SEQ ID NO: 518 - DNA, 21 bp, Artificial Sequence
  Synthetic Oligonucleotide
aggacgtattcacaatcactg

SEQ ID NO: 519 - DNA, 27 bp, Artificial Sequence
  Synthetic Oligonucleotide
ataatcattcacctccatcattcataa

SEQ ID NO: 520 - DNA, 20 bp, Artificial Sequence
  Synthetic Oligonucleotide
actgaatatggttcgtctca

SEQ ID NO: 521 - DNA, 25 bp, Artificial Sequence
  Synthetic Oligonucleotide
gtacattcacatatagaccatctta

SEQ ID NO: 522 - DNA, 22 bp, Artificial Sequence
  Synthetic Oligonucleotide
acataggtgcaggtagaatagt

SEQ ID NO: 523 - DNA, 20 bp, Artificial Sequence
  Synthetic Oligonucleotide
actccaccaggatgttgtcc

SEQ ID NO: 524 - DNA, 20 bp, Artificial Sequence
  Synthetic Oligonucleotide
gtaggaccgtcgtgtccaag

SEQ ID NO: 525 - DNA, 28 bp, Artificial Sequence
  Synthetic Oligonucleotide
gcaatatcaatggtatcgaaggcactat

SEQ ID NO: 526 - DNA, 27 bp, Artificial Sequence
  Synthetic Oligonucleotide
gtattgaaggtactattagcgatatgc

SEQ ID NO: 527 - DNA, 23 bp, Artificial Sequence
  Synthetic Oligonucleotide
gtgccggtctcggttactcaatg

SEQ ID NO: 528 - DNA, 24 bp, Artificial Sequence
  Synthetic Oligonucleotide
ggattattataatgcagctagaag

SEQ ID NO: 529 - DNA, 24 bp, Artificial Sequence
  Synthetic Oligonucleotide
gtacattcacatatagaccatctt

SEQ ID NO: 530 - DNA, 23 bp, Artificial Sequence
  Synthetic Oligonucleotide
acataggtgcaggtagaatagta

SEQ ID NO: 531 - DNA, 28 bp, Artificial Sequence
  Synthetic Oligonucleotide
agttccttcatatgactcagttgattga

SEQ ID NO: 532 - DNA, 27 bp, Artificial Sequence
  Synthetic Oligonucleotide
gttatatcttcaattatacattcctgc

SEQ ID NO: 533 - DNA, 23 bp, Artificial Sequence
  Synthetic Oligonucleotide
cagcagttgttgctagaggtatg

SEQ ID NO: 534 - DNA, 21 bp, Artificial Sequence
  Synthetic Oligonucleotide
gcatcaccaggtgcagcaagt

SEQ ID NO: 535 - DNA, 23 bp, Artificial Sequence
  Synthetic Oligonucleotide
agtggtgaaggtgttcaacaagc

SEQ ID NO: 536 - DNA, 23 bp, Artificial Sequence
  Synthetic Oligonucleotide
actgaagctggatatgttggaga

SEQ ID NO: 537 - DNA, 28 bp, Artificial Sequence
  Synthetic Oligonucleotide
gcaattctctgttgttgtcctccactca

SEQ ID NO: 538 - DNA, 25 bp, Artificial Sequence
  Synthetic Oligonucleotide
agtaagagcctcttcttggtcatga

SEQ ID NO: 539 - DNA, 28 bp, Artificial Sequence
  Synthetic Oligonucleotide
ctattcctgataataagtgtgtcctcat

SEQ ID NO: 540 - DNA, 23 bp, Artificial Sequence
  Synthetic Oligonucleotide
cggcatcatctaacaattcttct

SEQ ID NO: 541 - DNA, 28 bp, Artificial Sequence
  Synthetic Oligonucleotide
gtaattccaattacttctagctctggtg

SEQ ID NO: 542 - DNA, 21 bp, Artificial Sequence
  Synthetic Oligonucleotide
taccatcttctccatgtgtat

SEQ ID NO: 543 - DNA, 22 bp, Artificial Sequence
  Synthetic Oligonucleotide
ccatgcagatacaatgaaccag

SEQ ID NO: 544 - DNA, 23 bp, Artificial Sequence
  Synthetic Oligonucleotide
gatgataagacacatccaattcc

SEQ ID NO: 545 - DNA, 28 bp, Artificial Sequence
  Synthetic Oligonucleotide
ccttctgccattgtagaacaagctccat

SEQ ID NO: 546 - DNA, 20 bp, Artificial Sequence
  Synthetic Oligonucleotide
cctgtaactgtccactgagc

SEQ ID NO: 547 - DNA, 25 bp, Artificial Sequence
  Synthetic Oligonucleotide
caatcatgatagaattagatggaac

SEQ ID NO: 548 - DNA, 24 bp, Artificial Sequence
  Synthetic Oligonucleotide
agcaatagttccatcaggagcatc

SEQ ID NO: 549 - DNA, 22 bp, Artificial Sequence
  Synthetic Oligonucleotide
agtggtgaaggtgttcaacaag

SEQ ID NO: 550 - DNA, 22 bp, Artificial Sequence
  Synthetic Oligonucleotide
actgaagctggatatgttggag

SEQ ID NO: 551 - DNA, 23 bp, Artificial Sequence
  Synthetic Oligonucleotide
cgcctcttcagaagcggatatca

SEQ ID NO: 552 - DNA, 21 bp, Artificial Sequence
  Synthetic Oligonucleotide
gccagacttccgccacaacct

SEQ ID NO: 553 - DNA, 25 bp, Artificial Sequence
  Synthetic Oligonucleotide
ggcataataggatggatagatgagc

SEQ ID NO: 554 - DNA, 23 bp, Artificial Sequence
  Synthetic Oligonucleotide
gcagcagttgtacctacaactaa

SEQ ID NO: 555 - DNA, 27 bp, Artificial Sequence
  Synthetic Oligonucleotide
agttccttcatatgactcagttgattg

SEQ ID NO: 556 - DNA, 28 bp, Artificial Sequence
  Synthetic Oligonucleotide
gttatatcttcaattatacattcctgcg

SEQ ID NO: 557 - DNA, 27 bp, Artificial Sequence
  Synthetic Oligonucleotide
gcatggtagttcgccagccgctggaac

SEQ ID NO: 558 - DNA, 24 bp, Artificial Sequence
  Synthetic Oligonucleotide
acagcaaccgcaagttcttgacat

SEQ ID NO: 559 - DNA, 28 bp, Artificial Sequence
  Synthetic Oligonucleotide
aatatcatggtcgtgtccaggcactggc

SEQ ID NO: 560 - DNA, 24 bp, Artificial Sequence
  Synthetic Oligonucleotide
gttctggtagctgcttctactgta

SEQ ID NO: 561 - DNA, 27 bp, Artificial Sequence
  Synthetic Oligonucleotide
aacttacaactacgcgcacttgaatcg

SEQ ID NO: 562 - DNA, 23 bp, Artificial Sequence
  Synthetic Oligonucleotide
gagtgttgtatgatagtctcggt

SEQ ID NO: 563 - DNA, 28 bp, Artificial Sequence
  Synthetic Oligonucleotide
gcaagttgaggagatgctggcatgattc

SEQ ID NO: 564 - DNA, 26 bp, Artificial Sequence
  Synthetic Oligonucleotide
acatggctctggaagatgtgctgatc

SEQ ID NO: 565 - DNA, 28 bp, Artificial Sequence
  Synthetic Oligonucleotide
gcgataattgtaatgattcgtggtgtta

SEQ ID NO: 566 - DNA, 25 bp, Artificial Sequence
  Synthetic Oligonucleotide
ccgttgtcaatccagttagtagact

SEQ ID NO: 567 - DNA, 27 bp, Artificial Sequence
  Synthetic Oligonucleotide
actgtggcagtctatgttccaattgta

SEQ ID NO: 568 - DNA, 24 bp, Artificial Sequence
  Synthetic Oligonucleotide
cttatcgacataatcctgataatc

SEQ ID NO: 569 - DNA, 21 bp, Artificial Sequence
  Synthetic Oligonucleotide
gcgtcgcttcttgcgctcgcc

SEQ ID NO: 570 - DNA, 21 bp, Artificial Sequence
  Synthetic Oligonucleotide
aatgtattcataccgtcaagt

SEQ ID NO: 571 - DNA, 28 bp, Artificial Sequence
  Synthetic Oligonucleotide
gccttcacaactacgttggaaggtcttc

SEQ ID NO: 572 - DNA, 20 bp, Artificial Sequence
  Synthetic Oligonucleotide
ctaacagtcctgccgactac

SEQ ID NO: 573 - DNA, 27 bp, Artificial Sequence
  Synthetic Oligonucleotide
gccttcacaactacgttggaaggtctt

SEQ ID NO: 574 - DNA, 21 bp, Artificial Sequence
  Synthetic Oligonucleotide
ctaacagtcctgccgactact

SEQ ID NO: 575 - DNA, 27 bp, Artificial Sequence
  Synthetic Oligonucleotide
gccgctgagcggcggcaagccgatggc

SEQ ID NO: 576 - DNA, 24 bp, Artificial Sequence
  Synthetic Oligonucleotide
gaatggcaggccaagctgaaggcg

SEQ ID NO: 577 - DNA, 27 bp, Artificial Sequence
  Synthetic Oligonucleotide
gccaagcggcattctggcgccagtgga

SEQ ID NO: 578 - DNA, 28 bp, Artificial Sequence
  Synthetic Oligonucleotide
ccagaccggagtggacaacgtcgaggcg

SEQ ID NO: 579 - DNA, 28 bp, Artificial Sequence
  Synthetic Oligonucleotide
gccgtatatcatcggcaataaccgcacg

SEQ ID NO: 580 - DNA, 21 bp, Artificial Sequence
  Synthetic Oligonucleotide
gcatgatggtcaacaaggtgc

SEQ ID NO: 581 - DNA, 27 bp, Artificial Sequence
  Synthetic Oligonucleotide
acgagccgagataggtctgcagcgtac

SEQ ID NO: 582 - DNA, 24 bp, Artificial Sequence
  Synthetic Oligonucleotide
gtactgatattcaccatactgccg

SEQ ID NO: 583 - DNA, 27 bp, Artificial Sequence
  Synthetic Oligonucleotide
gcaatatcttcaccggcagccaccgcg

SEQ ID NO: 584 - DNA, 21 bp, Artificial Sequence
  Synthetic Oligonucleotide
ggtatatggcacgccaatcgc

SEQ ID NO: 585 - DNA, 24 bp, Artificial Sequence
  Synthetic Oligonucleotide
aataaccttaacgtcgccaacacg

SEQ ID NO: 586 - DNA, 23 bp, Artificial Sequence
  Synthetic Oligonucleotide
ctcggtgaacacctcctggcacg

SEQ ID NO: 587 - DNA, 24 bp, Artificial Sequence
  Synthetic Oligonucleotide
gcggaactgcttggcgtagtaagc

SEQ ID NO: 588 - DNA, 25 bp, Artificial Sequence
  Synthetic Oligonucleotide
catgtagtgccgtagaccttcacca

SEQ ID NO: 589 - DNA, 28 bp, Artificial Sequence
  Synthetic Oligonucleotide
gcgagaccggcggcaccatcgtctccag

SEQ ID NO: 590 - DNA, 27 bp, Artificial Sequence
  Synthetic Oligonucleotide
ttctgcctgatggacgtctccggctcg

SEQ ID NO: 591 - DNA, 28 bp, Artificial Sequence
  Synthetic Oligonucleotide
gcggttcacctgttcgccttcgaacacg

SEQ ID NO: 592 - DNA, 28 bp, Artificial Sequence
  Synthetic Oligonucleotide
gcgcagcatctgacgcaggatggtctcg

SEQ ID NO: 593 - DNA, 28 bp, Artificial Sequence
  Synthetic Oligonucleotide
actccatcgccatcaaggacatggccgg

SEQ ID NO: 594 - DNA, 27 bp, Artificial Sequence
  Synthetic Oligonucleotide
atcgacgtgttccgcatcttcgacgcg

SEQ ID NO: 595 - DNA, 23 bp, Artificial Sequence
  Synthetic Oligonucleotide
gcctgatgcactacagcgcctgg

SEQ ID NO: 596 - DNA, 27 bp, Artificial Sequence
  Synthetic Oligonucleotide
taccacatggtcgatctcgacgactgc

SEQ ID NO: 597 - DNA, 23 bp, Artificial Sequence
  Synthetic Oligonucleotide
gcgcatccaggacggcgagtacg

SEQ ID NO: 598 - DNA, 24 bp, Artificial Sequence
  Synthetic Oligonucleotide
cttcgagtgcctgcacgagctgaa

SEQ ID NO: 599 - DNA, 28 bp, Artificial Sequence
  Synthetic Oligonucleotide
gctggagaacgtcaaggtggtgatcatc

SEQ ID NO: 600 - DNA, 23 bp, Artificial Sequence
  Synthetic Oligonucleotide
accgataacgacgaccgcatcaa

SEQ ID NO: 601 - DNA, 26 bp, Artificial Sequence
  Synthetic Oligonucleotide
acgattggagaaggcagtgtgattgg

SEQ ID NO: 602 - DNA, 19 bp, Artificial Sequence
  Synthetic Oligonucleotide
ggacagattacaattggcg

SEQ ID NO: 603 - DNA, 20 bp, Artificial Sequence
  Synthetic Oligonucleotide
gccgcaataccgatattcca

SEQ ID NO: 604 - DNA, 22 bp, Artificial Sequence
  Synthetic Oligonucleotide
ccattgtccaccagctgaaccg

SEQ ID NO: 605 - DNA, 23 bp, Artificial Sequence
  Synthetic Oligonucleotide
gtgaaggtcgtgctcctatcggt

SEQ ID NO: 606 - DNA, 23 bp, Artificial Sequence
  Synthetic Oligonucleotide
agatctggtgaagttcgtatgat

SEQ ID NO: 607 - DNA, 23 bp, Artificial Sequence
  Synthetic Oligonucleotide
gctggtacttgtacttatatcga

SEQ ID NO: 608 - DNA, 26 bp, Artificial Sequence
  Synthetic Oligonucleotide
atcagaagatgatatcgttacgtcat

SEQ ID NO: 609 - DNA, 27 bp, Artificial Sequence
  Synthetic Oligonucleotide
gcgcatattgcattaatggctatagat

SEQ ID NO: 610 - DNA, 20 bp, Artificial Sequence
  Synthetic Oligonucleotide
gccagcaggttatacactcg

SEQ ID NO: 611 - DNA, 28 bp, Artificial Sequence
  Synthetic Oligonucleotide
gcaattcttaccacagcacgaagaacag

SEQ ID NO: 612 - DNA, 24 bp, Artificial Sequence
  Synthetic Oligonucleotide
atctagatgaagataatgaagtcg

SEQ ID NO: 613 - DNA, 28 bp, Artificial Sequence
  Synthetic Oligonucleotide
gcatcttcatacaatacttctagcttac

SEQ ID NO: 614 - DNA, 21 bp, Artificial Sequence
  Synthetic Oligonucleotide
cacaataccagttgtattacg

SEQ ID NO: 615 - DNA, 27 bp, Artificial Sequence
  Synthetic Oligonucleotide
gcttcagcgccattaccgccaccagct

SEQ ID NO: 616 - DNA, 24 bp, Artificial Sequence
  Synthetic Oligonucleotide
actcttgatatattcttgtaagcg

SEQ ID NO: 617 - DNA, 27 bp, Artificial Sequence
  Synthetic Oligonucleotide
gttcacacaacgcgccgactagaatcc

SEQ ID NO: 618 - DNA, 27 bp, Artificial Sequence
  Synthetic Oligonucleotide
cacgatatccaagataatgattggcta

SEQ ID NO: 619 - DNA, 27 bp, Artificial Sequence
  Synthetic Oligonucleotide
gcgcacctacaatcgccattactacac

SEQ ID NO: 620 - DNA, 28 bp, Artificial Sequence
  Synthetic Oligonucleotide
actcattatcgactgttacatcgactga

SEQ ID NO: 621 - DNA, 27 bp, Artificial Sequence
  Synthetic Oligonucleotide
agcgcacatgtgacagcgtgtaggtta

SEQ ID NO: 622 - DNA, 24 bp, Artificial Sequence
  Synthetic Oligonucleotide
gtgccttagattgttcagaacaat

SEQ ID NO: 623 - DNA, 28 bp, Artificial Sequence
  Synthetic Oligonucleotide
cgaatggatatgtaccatggtcgatatc

SEQ ID NO: 624 - DNA, 20 bp, Artificial Sequence
  Synthetic Oligonucleotide
ctctctaatatgatgtccat

SEQ ID NO: 625 - DNA, 28 bp, Artificial Sequence
  Synthetic Oligonucleotide
actacaacagcaaccgcattacaatggc

SEQ ID NO: 626 - DNA, 20 bp, Artificial Sequence
  Synthetic Oligonucleotide
ggtgctaagaggtcatcgga

SEQ ID NO: 627 - DNA, 24 bp, Artificial Sequence
  Synthetic Oligonucleotide
agcttcagataagtacctatctga

SEQ ID NO: 628 - DNA, 28 bp, Artificial Sequence
  Synthetic Oligonucleotide
ggaagaatagttattcttgataatgtat

SEQ ID NO: 629 - DNA, 22 bp, Artificial Sequence
  Synthetic Oligonucleotide
cgtattgctcgaatacatgata

SEQ ID NO: 630 - DNA, 20 bp, Artificial Sequence
  Synthetic Oligonucleotide
acaatgtatcaaggccagct

SEQ ID NO: 631 - DNA, 24 bp, Artificial Sequence
  Synthetic Oligonucleotide
gcgaccagttgttatcgaccgtgt

SEQ ID NO: 632 - DNA, 22 bp, Artificial Sequence
  Synthetic Oligonucleotide
cagaacgatacggtgctgtata

SEQ ID NO: 633 - DNA, 28 bp, Artificial Sequence
  Synthetic Oligonucleotide
caattacattgtctgttgcgtagatacc

SEQ ID NO: 634 - DNA, 21 bp, Artificial Sequence
  Synthetic Oligonucleotide
gttgtggctaatgtgccagtt

SEQ ID NO: 635 - DNA, 27 bp, Artificial Sequence
  Synthetic Oligonucleotide
gcaccactctatagcagtagcgtattg

SEQ ID NO: 636 - DNA, 25 bp, Artificial Sequence
  Synthetic Oligonucleotide
acagccaatgtcacctaagtcaaca

SEQ ID NO: 637 - DNA, 28 bp, Artificial Sequence
  Synthetic Oligonucleotide
acagtccgaataagatacgactattcga

SEQ ID NO: 638 - DNA, 25 bp, Artificial Sequence
  Synthetic Oligonucleotide
cgttgtaacgtatatgaatagttga

SEQ ID NO: 639 - DNA, 28 bp, Artificial Sequence
  Synthetic Oligonucleotide
agatgcaataacaggtcgaatattaatt

SEQ ID NO: 640 - DNA, 20 bp, Artificial Sequence
  Synthetic Oligonucleotide
gccatagtgagagtagtgaa

SEQ ID NO: 641 - DNA, 28 bp, Artificial Sequence
  Synthetic Oligonucleotide
caataacaggtcgaatattaattaattg

SEQ ID NO: 642 - DNA, 21 bp, Artificial Sequence
  Synthetic Oligonucleotide
gccatagtgagagtagtgaac

SEQ ID NO: 643 - DNA, 26 bp, Artificial Sequence
  Synthetic Oligonucleotide
agatgcaataacaggtcgaatattaa

SEQ ID NO: 644 - DNA, 22 bp, Artificial Sequence
  Synthetic Oligonucleotide
acacatacggccatagtgagag

SEQ ID NO: 645 - DNA, 24 bp, Artificial Sequence
  Synthetic Oligonucleotide
gaacataacgcgacgttccagctg

SEQ ID NO: 646 - DNA, 21 bp, Artificial Sequence
  Synthetic Oligonucleotide
gcttcagaggtgttgtagtcg

SEQ ID NO: 647 - DNA, 25 bp, Artificial Sequence
  Synthetic Oligonucleotide
gcgctggcgcagtatcgtgaactgg

SEQ ID NO: 648 - DNA, 22 bp, Artificial Sequence
  Synthetic Oligonucleotide
accaacgtaatctctattaccg

SEQ ID NO: 649 - DNA, 28 bp, Artificial Sequence
  Synthetic Oligonucleotide
gctgtaatgcaagtagcgtatgcgctca

SEQ ID NO: 650 - DNA, 20 bp, Artificial Sequence
  Synthetic Oligonucleotide
aaggccgccaatgcctgacg

SEQ ID NO: 651 - DNA, 27 bp, Artificial Sequence
  Synthetic Oligonucleotide
gcctgtagcaacagtaccacgaccagt

SEQ ID NO: 652 - DNA, 21 bp, Artificial Sequence
  Synthetic Oligonucleotide
caccacgtaataatgcaccaa

SEQ ID NO: 653 - DNA, 27 bp, Artificial Sequence
  Synthetic Oligonucleotide
actacgctgaagctggtgacaacattg

SEQ ID NO: 654 - DNA, 21 bp, Artificial Sequence
  Synthetic Oligonucleotide
gttgaggacgtattctcaatc

SEQ ID NO: 655 - DNA, 20 bp, Artificial Sequence
  Synthetic Oligonucleotide
gctggtacttacgttcagat

SEQ ID NO: 656 - DNA, 21 bp, Artificial Sequence
  Synthetic Oligonucleotide
acggtgaacgccgttacatcc

SEQ ID NO: 657 - DNA, 22 bp, Artificial Sequence
  Synthetic Oligonucleotide
gcaattcttaccacagcacgaa

SEQ ID NO: 658 - DNA, 24 bp, Artificial Sequence
  Synthetic Oligonucleotide
atctagatgaagataatgaagtcg

SEQ ID NO: 659 - DNA, 23 bp, Artificial Sequence
  Synthetic Oligonucleotide
gcggcggcaggcggtaacgccag

SEQ ID NO: 660 - DNA, 21 bp, Artificial Sequence
  Synthetic Oligonucleotide
acgcggttatctaccacggcg

SEQ ID NO: 661 - DNA, 26 bp, Artificial Sequence
  Synthetic Oligonucleotide
gcacctacttgtccagcaccagccat

SEQ ID NO: 662 - DNA, 22 bp, Artificial Sequence
  Synthetic Oligonucleotide
aataccaccaccaatacaagca

SEQ ID NO: 663 - DNA, 24 bp, Artificial Sequence
  Synthetic Oligonucleotide
gcgcggtaacatgccatattctgc

SEQ ID NO: 664 - DNA, 20 bp, Artificial Sequence
  Synthetic Oligonucleotide
cctgaatgacatcacagtcg

SEQ ID NO: 665 - DNA, 23 bp, Artificial Sequence
  Synthetic Oligonucleotide
aatcaggtcaaggaactgcaagc

SEQ ID NO: 666 - DNA, 25 bp, Artificial Sequence
  Synthetic Oligonucleotide
gtctcaatcatatgcaccggaatac

SEQ ID NO: 667 - DNA, 26 bp, Artificial Sequence
  Synthetic Oligonucleotide
gaacatatgtgtatgacgatgcgcgg

SEQ ID NO: 668 - DNA, 27 bp, Artificial Sequence
  Synthetic Oligonucleotide
gtacatgtcgcttatctgccagaaggt

SEQ ID NO: 669 - DNA, 25 bp, Artificial Sequence
  Synthetic Oligonucleotide
cgtgtgcgtagtgacgagttggaga

SEQ ID NO: 670 - DNA, 27 bp, Artificial Sequence
  Synthetic Oligonucleotide
agaatacgatgatgtaaggtacaccta

SEQ ID NO: 671 - DNA, 21 bp, Artificial Sequence
  Synthetic Oligonucleotide
caggagttacttctgttccat

SEQ ID NO: 672 - DNA, 21 bp, Artificial Sequence
  Synthetic Oligonucleotide
ttgaacaattagatcacctcg

SEQ ID NO: 673 - DNA, 28 bp, Artificial Sequence
  Synthetic Oligonucleotide
cgtaatctccattaccgatggtcagatc

SEQ ID NO: 674 - DNA, 25 bp, Artificial Sequence
  Synthetic Oligonucleotide
acgtattctacctccactctcgtct

SEQ ID NO: 675 - DNA, 23 bp, Artificial Sequence
  Synthetic Oligonucleotide
cattcgacgttctggtattactt

SEQ ID NO: 676 - DNA, 27 bp, Artificial Sequence
  Synthetic Oligonucleotide
cacgctccgcatcagcagcaccacgtt

SEQ ID NO: 677 - DNA, 25 bp, Artificial Sequence
  Synthetic Oligonucleotide
ctgaaccacggattactggagtgtc

SEQ ID NO: 678 - DNA, 23 bp, Artificial Sequence
  Synthetic Oligonucleotide
gcctgttactactgtaccacgac

SEQ ID NO: 679 - DNA, 25 bp, Artificial Sequence
  Synthetic Oligonucleotide
gaatcgaacggtctcattaacagat

SEQ ID NO: 680 - DNA, 22 bp, Artificial Sequence
  Synthetic Oligonucleotide
gctttccagggatataagacgc

SEQ ID NO: 681 - DNA, 20 bp, Artificial Sequence
  Synthetic Oligonucleotide
cccgcagagtcacactcgga

SEQ ID NO: 682 - DNA, 22 bp, Artificial Sequence
  Synthetic Oligonucleotide
actcttggtactactcactagc

SEQ ID NO: 683 - DNA, 27 bp, Artificial Sequence
  Synthetic Oligonucleotide
gagtctctttcaacctggattagatat

SEQ ID NO: 684 - DNA, 24 bp, Artificial Sequence
  Synthetic Oligonucleotide
aagattaatagcgtactttactcc

SEQ ID NO: 685 - DNA, 25 bp, Artificial Sequence
  Synthetic Oligonucleotide
atcccgcagatactaggttcttaat

SEQ ID NO: 686 - DNA, 25 bp, Artificial Sequence
  Synthetic Oligonucleotide
gaactattcatattacaccctaagg

SEQ ID NO: 687 - DNA, 23 bp, Artificial Sequence
  Synthetic Oligonucleotide
cagtgggctatcctaagccaaag

SEQ ID NO: 688 - DNA, 24 bp, Artificial Sequence
  Synthetic Oligonucleotide
cataagcgaactaactatcactta

SEQ ID NO: 689 - DNA, 26 bp, Artificial Sequence
  Synthetic Oligonucleotide
acaaagcgttctaaacgattagaact

SEQ ID NO: 690 - DNA, 23 bp, Artificial Sequence
  Synthetic Oligonucleotide
cgagaaaggaaacaggatagtac

SEQ ID NO: 691 - DNA, 25 bp, Artificial Sequence
  Synthetic Oligonucleotide
ccaatggagaagtctaaatgtccaa

SEQ ID NO: 692 - DNA, 25 bp, Artificial Sequence
  Synthetic Oligonucleotide
ttatcagagatacatgactcttagg

SEQ ID NO: 693 - DNA, 28 bp, Artificial Sequence
  Synthetic Oligonucleotide
cgaatcactggactacatttatatttct

SEQ ID NO: 694 - DNA, 23 bp, Artificial Sequence
  Synthetic Oligonucleotide
agcgaacctttatatttgaccat

SEQ ID NO: 695 - DNA, 26 bp, Artificial Sequence
  Synthetic Oligonucleotide
ctcaagtcttgccctgatagaattat

SEQ ID NO: 696 - DNA, 25 bp, Artificial Sequence
  Synthetic Oligonucleotide
tcacgacttatctactttagaaatc

SEQ ID NO: 697 - DNA, 27 bp, Artificial Sequence
  Synthetic Oligonucleotide
agtgttaggtctttattaattagccca

SEQ ID NO: 698 - DNA, 25 bp, Artificial Sequence
  Synthetic Oligonucleotide
tttgatttgcctattgagaaattaa

SEQ ID NO: 699 - DNA, 26 bp, Artificial Sequence
  Synthetic Oligonucleotide
ggtgatcgttattatgatagtacggc

SEQ ID NO: 700 - DNA, 21 bp, Artificial Sequence
  Synthetic Oligonucleotide
ctcggttaagggaattacgac

SEQ ID NO: 701 - DNA, 25 bp, Artificial Sequence
  Synthetic Oligonucleotide
actcggatggtaggtttattaaagc

SEQ ID NO: 702 - DNA, 24 bp, Artificial Sequence
  Synthetic Oligonucleotide
gtgatcgttattatgatagtacgg

SEQ ID NO: 703 - DNA, 22 bp, Artificial Sequence
  Synthetic Oligonucleotide
ggagcggtaacaagtttccacc

SEQ ID NO: 704 - DNA, 25 bp, Artificial Sequence
  Synthetic Oligonucleotide
ggaatattgttggatttaaagacaa

SEQ ID NO: 705 - DNA, 23 bp, Artificial Sequence
  Synthetic Oligonucleotide
acaatcgttgtcgcactgcatag

SEQ ID NO: 706 - DNA, 21 bp, Artificial Sequence
  Synthetic Oligonucleotide
gaacttggtctaccgtaccac

SEQ ID NO: 707 - DNA, 27 bp, Artificial Sequence
  Synthetic Oligonucleotide
ggataatacaatcctaatacgtacgga

SEQ ID NO: 708 - DNA, 20 bp, Artificial Sequence
  Synthetic Oligonucleotide
gctgctgtaactagggtagc

SEQ ID NO: 709 - DNA, 24 bp, Artificial Sequence
  Synthetic Oligonucleotide
ctatattcaacgggtcacgggtag

SEQ ID NO: 710 - DNA, 23 bp, Artificial Sequence
  Synthetic Oligonucleotide
tcattgattcgatctcgtaactc

SEQ ID NO: 711 - DNA, 24 bp, Artificial Sequence
  Synthetic Oligonucleotide
aatgttattgtggttgcgtgttcg

SEQ ID NO: 712 - DNA, 20 bp, Artificial Sequence
  Synthetic Oligonucleotide
tactttggaagtgccctgac

SEQ ID NO: 713 - DNA, 25 bp, Artificial Sequence
  Synthetic Oligonucleotide
catgtcttctagtacaggtttgccg

SEQ ID NO: 714 - DNA, 20 bp, Artificial Sequence
  Synthetic Oligonucleotide
tgtaagaggccgctaacttc

SEQ ID NO: 715 - DNA, 19 bp, Artificial Sequence
  Synthetic Oligonucleotide
ctctggctcgtgggctcgg

SEQ ID NO: 716 - DNA, 24 bp, Artificial Sequence
  Synthetic Oligonucleotide
ttcttgagatagtccggtataatc

SEQ ID NO: 717 - DNA, 21 bp, Artificial Sequence
  Synthetic Oligonucleotide
attcgatcacgatgggctggg

SEQ ID NO: 718 - DNA, 21 bp, Artificial Sequence
  Synthetic Oligonucleotide
aatttcctgtgtcatacacgc

SEQ ID NO: 719 - DNA, 26 bp, Artificial Sequence
  Synthetic Oligonucleotide
caattgatttagccactacaccttac

SEQ ID NO: 720 - DNA, 19 bp, Artificial Sequence
  Synthetic Oligonucleotide
cactattctggcgaccacc

SEQ ID NO: 721 - DNA, 24 bp, Artificial Sequence
  Synthetic Oligonucleotide
gataaagaagcgtcttgacccagt

SEQ ID NO: 722 - DNA, 19 bp, Artificial Sequence
  Synthetic Oligonucleotide
atctggtgctccttgacgc

SEQ ID NO: 723 - DNA, 24 bp, Artificial Sequence
  Synthetic Oligonucleotide
gcaaatttagagagtgcatgcatg

SEQ ID NO: 724 - DNA, 20 bp, Artificial Sequence
  Synthetic Oligonucleotide
ggaagaggacggcatacaac

SEQ ID NO: 725 - DNA, 23 bp, Artificial Sequence
  Synthetic Oligonucleotide
catttcatctagaccgctcgtgt

SEQ ID NO: 726 - DNA, 21 bp, Artificial Sequence
  Synthetic Oligonucleotide
gcttgaagtgtatgttgggac

SEQ ID NO: 727 - DNA, 19 bp, Artificial Sequence
  Synthetic Oligonucleotide
gtcgccctcgtgctaacgt

SEQ ID NO: 728 - DNA, 20 bp, Artificial Sequence
  Synthetic Oligonucleotide
ggttctttgatgtaccggtt

SEQ ID NO: 729 - DNA, 23 bp, Artificial Sequence
  Synthetic Oligonucleotide
gctgatgacggtgaagtttatca

SEQ ID NO: 730 - DNA, 22 bp, Artificial Sequence
  Synthetic Oligonucleotide
cattatcgcacatattgaccac

SEQ ID NO: 731 - DNA, 24 bp, Artificial Sequence
  Synthetic Oligonucleotide
gaaattagctaaagggatatcgcg

SEQ ID NO: 732 - DNA, 19 bp, Artificial Sequence
  Synthetic Oligonucleotide
aactttccgccaatcctgc

SEQ ID NO: 733 - DNA, 21 bp, Artificial Sequence
  Synthetic Oligonucleotide
cacctacgttctcacctgcac

SEQ ID NO: 734 - DNA, 22 bp, Artificial Sequence
  Synthetic Oligonucleotide
attcgatagtaccagttacgtc

SEQ ID NO: 735 - DNA, 21 bp, Artificial Sequence
  Synthetic Oligonucleotide
gttgcttatagcgtcgctgct

SEQ ID NO: 736 - DNA, 22 bp, Artificial Sequence
  Synthetic Oligonucleotide
ctggttatcgagaagataaagg

SEQ ID NO: 737 - DNA, 22 bp, Artificial Sequence
  Synthetic Oligonucleotide
gtaagcgtagcgatacgttgag

SEQ ID NO: 738 - DNA, 18 bp, Artificial Sequence
  Synthetic Oligonucleotide
gagtgaacgcaccactgg

SEQ ID NO: 739 - DNA, 23 bp, Artificial Sequence
  Synthetic Oligonucleotide
tcaggtagagaatactcaggcgc

SEQ ID NO: 740 - DNA, 19 bp, Artificial Sequence
  Synthetic Oligonucleotide
cggagaaggctaggttgtc

SEQ ID NO: 741 - DNA, 20 bp, Artificial Sequence
  Synthetic Oligonucleotide
gcaacccactcccatggtgt

SEQ ID NO: 742 - DNA, 21 bp, Artificial Sequence
  Synthetic Oligonucleotide
cgttcttcatcagacaatctg

SEQ ID NO: 743 - DNA, 24 bp, Artificial Sequence
  Synthetic Oligonucleotide
gccctttcaggactttgatactgg

SEQ ID NO: 744 - DNA, 21 bp, Artificial Sequence
  Synthetic Oligonucleotide
tgtacggagacggagttatcg

SEQ ID NO: 745 - DNA, 23 bp, Artificial Sequence
  Synthetic Oligonucleotide
acactgaccgattcatcctcgtg

SEQ ID NO: 746 - DNA, 21 bp, Artificial Sequence
  Synthetic Oligonucleotide
cttgaaagtgcgttaacaacc

SEQ ID NO: 747 - DNA, 22 bp, Artificial Sequence
  Synthetic Oligonucleotide
cggaagcccaccaagtgagtac

SEQ ID NO: 748 - DNA, 22 bp, Artificial Sequence
  Synthetic Oligonucleotide
cgaaaccagtttgtccttagtc

SEQ ID NO: 749 - DNA, 24 bp, Artificial Sequence
  Synthetic Oligonucleotide
accagcttgtctttagtctgagag

SEQ ID NO: 750 - DNA, 21 bp, Artificial Sequence
  Synthetic Oligonucleotide
ctttacgacgggtcatttcac

SEQ ID NO: 751 - DNA, 25 bp, Artificial Sequence
  Synthetic Oligonucleotide
cattggtttgttctgtttgagaggc

SEQ ID NO: 752 - DNA, 23 bp, Artificial Sequence
  Synthetic Oligonucleotide
gattcatcttcgtgaattgtgac

SEQ ID NO: 753 - DNA, 25 bp, Artificial Sequence
  Synthetic Oligonucleotide
ggactttgatactggaggagtcata

SEQ ID NO: 754 - DNA, 21 bp, Artificial Sequence
  Synthetic Oligonucleotide
tgtacggaaacggagttatcg

SEQ ID NO: 755 - DNA, 22 bp, Artificial Sequence
  Synthetic Oligonucleotide
atgctggaggagtcgtacgttt

SEQ ID NO: 756 - DNA, 21 bp, Artificial Sequence
  Synthetic Oligonucleotide
gtcgcgcacactaatagattc

SEQ ID NO: 757 - DNA, 25 bp, Artificial Sequence
  Synthetic Oligonucleotide
aactaaacctacacggaattggttc

SEQ ID NO: 758 - DNA, 21 bp, Artificial Sequence
  Synthetic Oligonucleotide
gcagatacacgacgtttatgt

SEQ ID NO: 759 - DNA, 22 bp, Artificial Sequence
  Synthetic Oligonucleotide
gccgcttcacctacgttaggaa

SEQ ID NO: 760 - DNA, 23 bp, Artificial Sequence
  Synthetic Oligonucleotide
cgtaaagatgagtctttaacgtc

SEQ ID NO: 761 - DNA, 23 bp, Artificial Sequence
  Synthetic Oligonucleotide
gacgtttgtgcgtaatctcagac

SEQ ID NO: 762 - DNA, 21 bp, Artificial Sequence
  Synthetic Oligonucleotide
gaggaaaccgtattcgttcgt

SEQ ID NO: 763 - DNA, 24 bp, Artificial Sequence
  Synthetic Oligonucleotide
acaacactttaccacttgagtggg

SEQ ID NO: 764 - DNA, 22 bp, Artificial Sequence
  Synthetic Oligonucleotide
gtaactgcccatgtcaagatac

SEQ ID NO: 765 - DNA, 22 bp, Artificial Sequence
  Synthetic Oligonucleotide
ccacgtttagttgaaccaccgc

SEQ ID NO: 766 - DNA, 22 bp, Artificial Sequence
  Synthetic Oligonucleotide
tcaatacgccagttgttagttc

SEQ ID NO: 767 - DNA, 25 bp, Artificial Sequence
  Synthetic Oligonucleotide
aatcgataataagtacggtgcatcc

SEQ ID NO: 768 - DNA, 22 bp, Artificial Sequence
  Synthetic Oligonucleotide
gaagaatacattcgcgtacatc

SEQ ID NO: 769 - DNA, 25 bp, Artificial Sequence
  Synthetic Oligonucleotide
aagcaagatcgagtcttcatagttg

SEQ ID NO: 770 - DNA, 23 bp, Artificial Sequence
  Synthetic Oligonucleotide
gatatacacgatacctgattcgt

SEQ ID NO: 771 - DNA, 25 bp, Artificial Sequence
  Synthetic Oligonucleotide
ccgatattcatacgagaaggtacac

SEQ ID NO: 772 - DNA, 23 bp, Artificial Sequence
  Synthetic Oligonucleotide
cagtaactctattgtcaaacggt

SEQ ID NO: 773 - DNA, 23 bp, Artificial Sequence
  Synthetic Oligonucleotide
gtagtgagtcgggtgtacgtctc

SEQ ID NO: 774 - DNA, 22 bp, Artificial Sequence
  Synthetic Oligonucleotide
tcttcgatagcagacagatagt

SEQ ID NO: 775 - DNA, 24 bp, Artificial Sequence
  Synthetic Oligonucleotide
acctacacggaattggttctcagt

SEQ ID NO: 776 - DNA, 21 bp, Artificial Sequence
  Synthetic Oligonucleotide
gatacacgacgtttgtgtgta

SEQ ID NO: 777 - DNA, 24 bp, Artificial Sequence
  Synthetic Oligonucleotide
caacatcattagcttggtcgtggg

SEQ ID NO: 778 - DNA, 20 bp, Artificial Sequence
  Synthetic Oligonucleotide
ttgcgtgttaccaactcgtc

SEQ ID NO: 779 - DNA, 21 bp, Artificial Sequence
  Synthetic Oligonucleotide
cggcacgtccgaatcgtatca

SEQ ID NO: 780 - DNA, 20 bp, Artificial Sequence
  Synthetic Oligonucleotide
tcgtgtcccgtatatgttgg

SEQ ID NO: 781 - DNA, 24 bp, Artificial Sequence
  Synthetic Oligonucleotide
aatagaggcccacaagtcttgttc

SEQ ID NO: 782 - DNA, 20 bp, Artificial Sequence
  Synthetic Oligonucleotide
cgctctccactatgggtagt

SEQ ID NO: 783 - DNA, 26 bp, Artificial Sequence
  Synthetic Oligonucleotide
gctacattaatcactatggacagaca

SEQ ID NO: 784 - DNA, 21 bp, Artificial Sequence
  Synthetic Oligonucleotide
gatggtcgatctatcgtctct

SEQ ID NO: 785 - DNA, 25 bp, Artificial Sequence
  Synthetic Oligonucleotide
gaagtgttattcaaactttggtccc

SEQ ID NO: 786 - DNA, 21 bp, Artificial Sequence
  Synthetic Oligonucleotide
cttgaacccttggttcaaggt

(19) United States
(12) Patent Application Publication
(10) Pub. No.: US 2015/0344977 A1
(43) Pub. Date: Dec. 3, 2015

(54) Method And System For Detection Of An Organism

(71) Applicant: Pathogenica, Inc., Cambridge, MA (US)

(72) Inventor: Philip Alexander Rolfe, Newton, MA (US)

(21) Appl. No.: 14/355,408

(22) PCT Filed: Nov. 1, 2012
(86) PCT No.: PCT/US2012/063042

§ 371 Date: Apr. 30, 2014

Related U.S. Application Data
(60) Provisional application No. 61/554,129, filed on Nov. 1, 2011.
(60) Provisional application No. 61/608,558, filed on Mar. 8, 2012.

(51) Int. Cl. C12Q 1/70; C12Q 1/68

(52) U.S. Cl. CPC C12Q 1/708; C12Q 1/689; C12Q 2600/16; C12Q 2600/156

(57) ABSTRACT

Provided herein are systems and method of detecting an organism, such as a microbe, microorganism or pathogen. The system can comprise one or more probi for detecting a strain with high sensitivity. The system can also detect the strain within a short time frame.

### RELATED APPLICATIONS

This application claims the benefit of U.S. Provisional Application Nos. 61/554,129, filed on Nov. 1, 2011 and 61/608,558 filed Mar. 8, 2012.

The entire teachings of the above applications are incorporated herein by reference.

### BACKGROUND

Detection of different organisms is important in many applications, such as in clinical diagnosis (for example, detection of viruses, parasites, bacteria, fungus), clinical monitoring (for example, viral/bacterial load, pathogen biomarkers, biomarkers of a host or subject), environmental biosurveillance (for example, hospital acquired infections, biological agents, controlled genetically modified organisms), as well as, in biological safety (detection of contaminants or foreign organism in blood supply, biologic agents, food/water agriculture, livestock pathogen surveillance and breeding, genetically modified crop pathogen and breeding, biodefense such as large volume air/water supply, surface swabs, and rapid identification from blood samples). In many cases, it is advantageous for a single test to be able to detect a large number of organisms. For example, a sepsis test or a respiratory panel may detect dozens or even several hundred different species in order to provide a complete diagnostic in a single test. For surveillance applications, it is often useful to determine both the strain or substrain in addition to the species or genus; such detailed information allows epidemiologists or infection control officers to track the spread of an organism through a geographic area or healthcare facility.

Sequencing platforms such as the Ion Torrent PGM and Proton, the Illumina MiSeq and HiSeq, 454's GS and GSJr, and the PacBio RS can simultaneously sequence thousands to millions of DNA molecules. Sequencing DNA from a pathogen's genome can identify the pathogen at the genus or species level, reveal the strain or sub-strain, and can also provide information about virulence factors or drug resistances. Thus, sequencing offers the ability to combine current techniques for detection or drug resistance testing, such as culture and qPCR, with techniques for strain typing, such as pulsed-field gel electrophoresis (PFGE) and multilocus sequencing typing (MLST), into a single test.

A simple application of sequencing to organism detection sequences all of the DNA or RNA from a sample such as a nasal swab, wound swab, blood sample, aspirate, urine, sputum, environmental surface swab, etc. However, this simple approach incurs a high sequencing cost as much of the DNA may be from the host. To ensure reliable identification of pathogens at low levels compared to the host genome, a user must sequence tens or hundreds of millions of DNA fragments.

Whole-sample sequencing also incurs a high analysis cost in terms of computer time and requires substantial technician time and expertise to interpret. Mapping or aligning sequencing reads to a large database of known genomes is computationally intensive, as is assembling a genome de-novo. Furthermore, both processes are relatively error prone because of the large number of variables both in the process (sequencing read count, sequencing read quality), the analysis (algorithm, parameters, genome database content), and the sample (number of organisms present, strains present, relative quantities, total amount of DNA). While sequencing from a purified isolate avoids host genome contamination, it requires additional time and laboratory steps such as culture to acquire the isolate and it still requires the same expensive and difficult analysis steps. For example, annotating the functional significance of genes in a newly sequenced genome is difficult-even if a gene family can be identified based on approximate protein homology, SNPs or other changes in the DNA sequence may substantially increase or decrease the activity of the resulting protein. In other cases, mutations in regulatory regions may change the organism's phenotype. While many tools exist to assist in the assembly, annotation, and functional analysis of genome sequence, this task has not been automated a remains a critical hurdle to the adoption of whole genome or whole sample sequencing as a routine clinical tool.

A better method of identifying organisms, determining the strain, and detecting clinically relevant phenotypes uses DNA sequencing to interrogate only key fingerprint or signature regions in the pathogen's genome. These techniques use one of several methods to select for or enrich certain regions of the organisms' genomes and sequence only those regions. The selection or enrichment largely avoids sequencing host DNA and can also reduce the amount of pathogen DNA to be sequenced by a factor of 1,000 or more. Furthermore, by only sequencing selected regions, the analysis of the resulting sequencing reads is vastly simpler. Mapping to or assembly only small genomic regions can reduce the computer time required by a factor of 100-1,000. Likewise, the analysis of such data can be automated more easily because each region was included in the test because it has a known relationship between the DNA sequence and the result. For example, one region may be known to distinguish between two species while another region may be the catalytic domain of an antibiotic resistance gene.

While selective-sequencing approaches offer many advantages in cost and simplicity, they may produce erroneous results when critical nucleotides within the fingerprint regions are sequenced incorrectly or when those regions are mutated in the isolate in the sample relative to a reference sequence. Thus, a critical aspect of designing a selective-sequencing test to identify organisms in a sample is to determine the number of loci or number of informative nucleotides that must be sequenced to achieve a desired level of confidence in the result.

### SUMMARY

The present invention uses DNA sequencing to determine the sequence of three or more regions of an organism's genome to determine the identity of the organism. The methods of this invention allow the identity to be determined with high specificity even in face of sequencing errors and natural genomic variability. In some embodiments, any of several techniques may be used choose regions of one or more genomes to sequence and then one of several techniques may be used to sequence only or primarily only those chosen regions of the genome or genomes. In other embodiments, the complete genome may be sequenced and only selected regions analyzed. In preferred embodiments, the regions chosen for sequencing or analysis are selected to achieve at least 99% specificity in distinguishing any organism in the target set from any other organism. In another preferred embodiment, the regions chosen for sequencing or analysis are selected to achieve at least 99% specificity in distinguishing known strains of an organism from each other.

The organism can be a microbe, microorganism, or pathogen, such as a virus, bacterium, or fungus. In one embodiment, an organism is distinguished from another organism. In another embodiment, a strain, variant or subtype of the organism is distinguished from another strain, variant, or subtype of the same organism. In other embodiments, the invention simultaneously determines the species and strain or subtype of the organism or organisms in a sample. For example, a strain, variant or subtype of a virus can be distinguished from another strain, variant or subtype of the same virus.

For use in a clinical setting, the number of hands-on steps, the amount of hands-on time, and the number of purification steps required substantially determine the utility of the method; fewer steps, less time, and fewer purifications or reagent transfers generally yield a simpler method that can be adopted in a wider range of facilities and used by technicians with less training. Furthermore, fewer steps and fewer transfers allow for easier adoption of a protocol for use on liquid handling robots or in microfluidic devices. Thus, this invention provides a protocol that may be performed in a single Eppendorf tube or other vessel using only serial additions of the reagents provided by a kit followed by a single purification for an entire set of samples that have been processed in parallel.

Also provided herein is a method of stratifying a host into a therapeutic group. In one embodiment, the method comprises determining the identity of a non-host organism or pathogenic strain, variant, or subtype from the sequencing and stratifying the host into a therapeutic group based on the identity of the non-host organism or pathogenic strain, variant, or subtype. In another embodiment, the method further comprises determining the genotype of the host, such as from the same or different sample. The method can also further comprise detecting one or more additional organisms or pathogens, or additional strains, variants, or subtypes of the same pathogen. In one embodiment, the identification of two pathogens or non-host organisms places a host in a therapeutic group that differs from that of which only one non-host organism or pathogen is identified. In yet another embodiment, the identification of two pathogenic strains, variants, or subtypes places the host in a therapeutic group that differs from that of which only one pathogenic strain, variant or subtype is identified.

In evaluating sequencing-based tests, the terms specificity and sensitivity are used slightly differently than for binary tests such as qPCR, ELISA, etc. In sequencing-based tests, it is rare for sequencing reads to be returned when no organism is present; thus, traditional false-positives are rare. Instead, errors are typically (1) false negatives in which no organism is detected when an organism was present in the sample or (2) mis-identifications in which the test incorrectly labels an organism present in the sample. To describe sequencing-based tests, we use specificity to mean the fraction or percent of cases in which the organism is correctly identified when the test detects and organism and we use sensitivity to mean one minus the fraction (or 100 minus the percent) of cases in which the test returns “no organism present” when an organism was present in the sample.

### BRIEF DESCRIPTION OF THE DRAWINGS

FIG. 1: Selecting only the most informative genomic regions substantially reduces the analysis time. Full bacterial genomes are typically 1 MB to 5 MB in size; a database of the several thousand sequenced bacterial genomes would include several gigabases of sequence. A probeset can be applied in-silico to the full genome database to produce a vastly smaller database that contains only the sequence of the informative region. Given that a probe set may select 1 kb to 10 kb of sequence from each full genome, the resulting signature regions database will be roughly 1,000 times smaller than the full genomes database, potentially increasing the analysis speed by a similar factor. Note that not all probes work against all genomes and that certain probes may target multiple regions in a single genome. The in-silico application of the probes to the genomes database can be performed with standard sequence alignment tools such as Blast, Blat, Bowtie, SOAP, etc.

FIG. 2: Sequencing reads are analyzed in a two step process. In the first step, the portion of the sequencing read that comes from the probe or primer is aligned against the list of probe or primer sequences; this list typically contains hundreds or thousands of relatively short sequences (perhaps 20-40 bp each). In the second step, the remainder of the sequencing read is compared against the set of sequences that the probe was predicted to produce from the set of full genomes; this set may contain hundreds or perhaps thousands of sequences of varying length, but typically 100-300 bp. Both comparisons can be performed quickly using well known algorithms such as Needleman-Wunsch or Needleman-Wunsch with hashing.

FIG. 3: A molecular inversion probeset designed to detect 13 common bacterial pathogens and 15 common drug resistance genes was used to assay DNA isolated from 3 bacterial samples. The resulting sequencing libraries sequenced on the Ion Torrent PGM. Result analysis was automatically generated using a plugin analysis pipeline that reports species and strain identity, and in addition the resistance gene sequences detected. The figure depicts the resistance gene profiles for the 3 samples, and the readcount of sequences mapping to each resistance gene within each sample. This report demonstrates the ability to stratify samples by the resistance gene sequences they contain, for instance the co-presence of aminoglycoside, quaternary ammonium compound and blaVIM-4 Type Metallo-β-Lactamase resistance genes in sample A, or Erythromycin and Methicilin resistance with potential β-Lactamase resistance within samples B and C.

FIG. 4 illustrates the workflow from DNA extraction to output of pathogen identification processed from sequencing data. The sample capture method described here enables sample to result workflow to be achieved in 14.5 hours (allowing for a 200 base sequencing run on the Ion Torrent PGM sequencing platform).

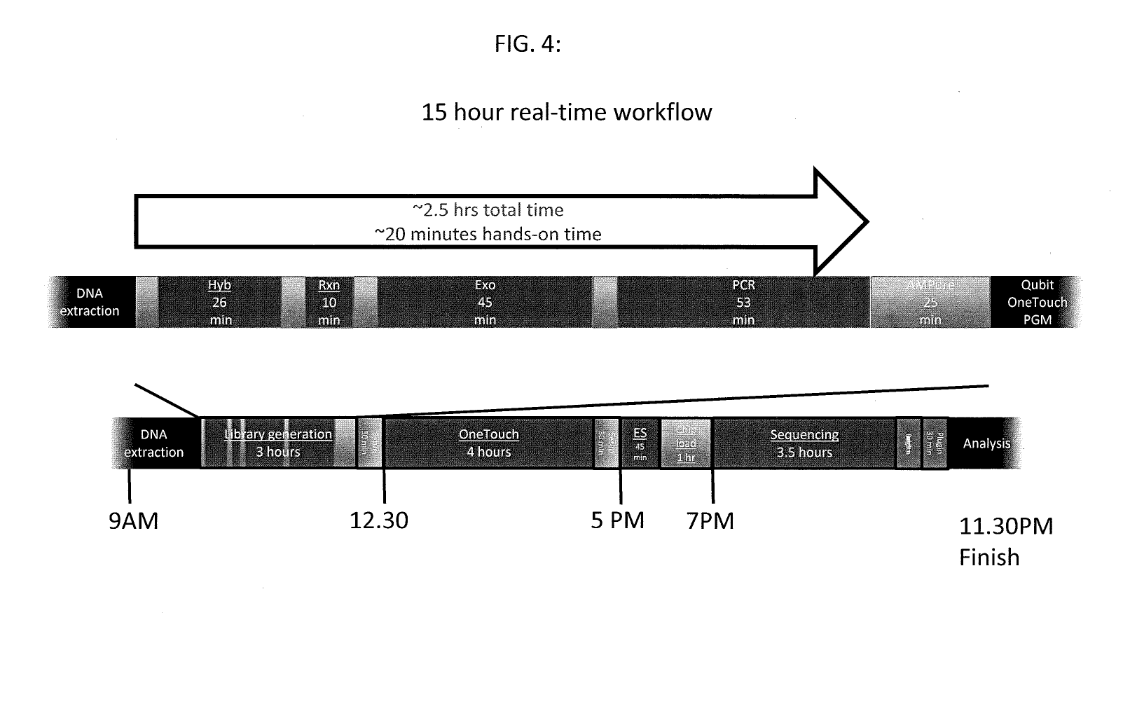

FIG. 5 summarizes results in an experiment where 21 samples of circulating nucleic acid <250 nt in size were extracted from human blood samples obtained from patients with active Hepatitis B infections. Additional control samples were generated at varying DNA concentrations using plasmids containing cloned regions of the HBV genome. The nucleic acid samples were contacted with molecular inversion probes targeting loci within the HBV viral genome, and circularized products generated were sequenced in duplicate on an Ion Torrent PGM sequencer. Readcounts per sample are recorded, alongside qPCR copy number determination using Sybr green and PCR primers to conserved regions of the HBV genome. The data demonstrates detection of circulating HBV fragments from blood to ˜10̂5 copies of target per sample, and broadly linear readcounts correlating with 10 fold dilutions of plasmid control samples.

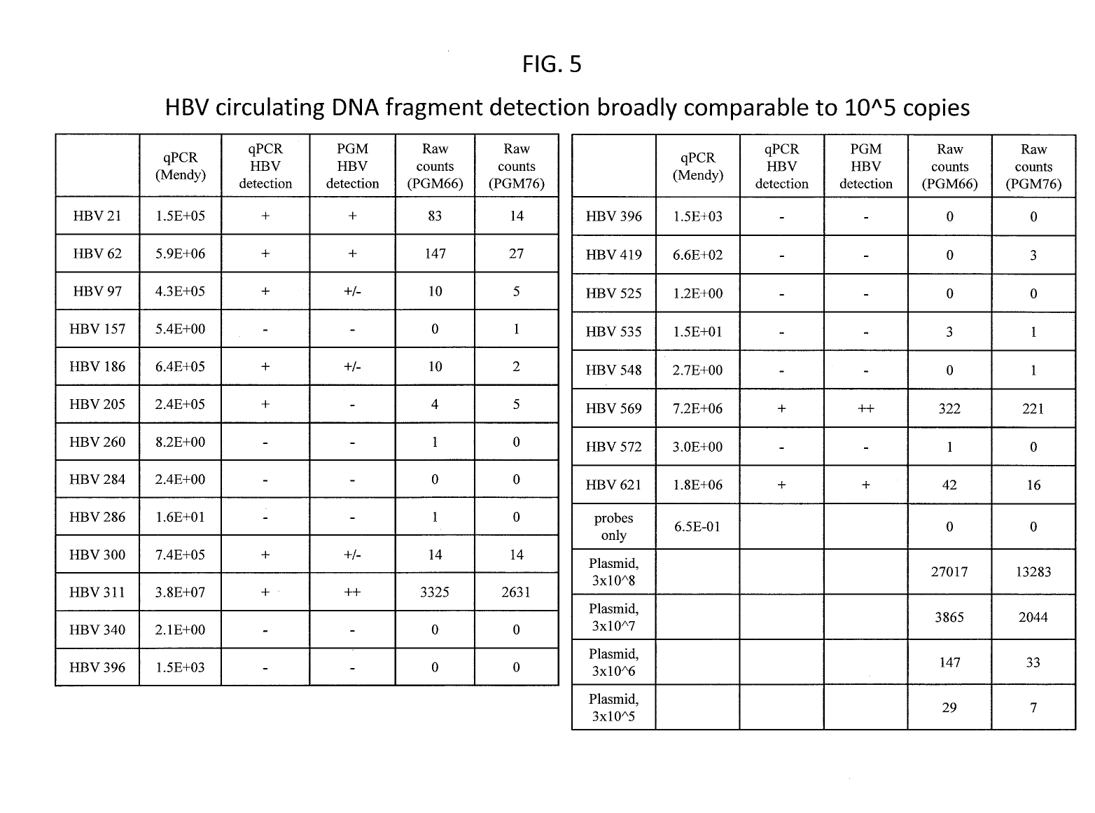

FIG. 6 Shows a table that records readcount generated from the assaying and sequencing of samples of circulating HBV DNA extracted from blood. Variant detection indicates the detection of amino acid codon variants that lead to a change in coding amino acid in the viral protein. % variant indicates the fraction of total circulating nucleic acid within an individual patient sample that contained a specifiedviral variant.

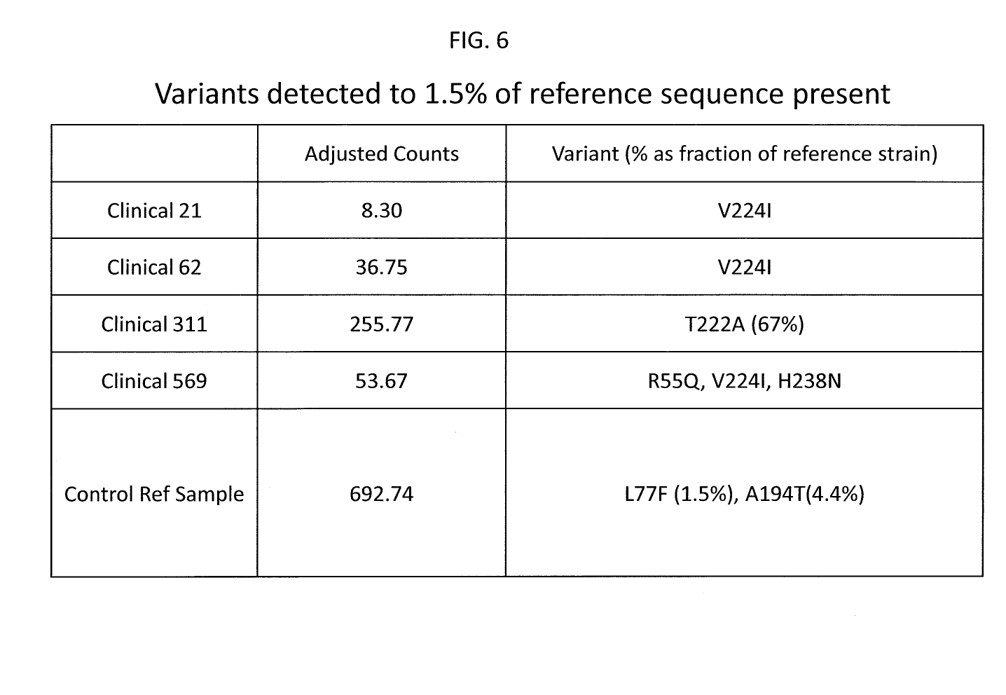

FIG. 7 Shows DNA from Nine Thinprep cervical brush samples were assayed using a molecular inversion probeset containing probes targeting 30 high-risk HPV variants, and the human TP53 gene locus. The combined probeset assay was performed in a single tube, and the sequencing libraries for each sample prepared and sequenced on the Ion Torrent PGM sequencer. The table records the identification of HPV viral subtypes present within each sample, and the nucleotide sequence of ˜a dozen SNPs in the TP53 gene for the individual from which the cervical brush sample was acquired.

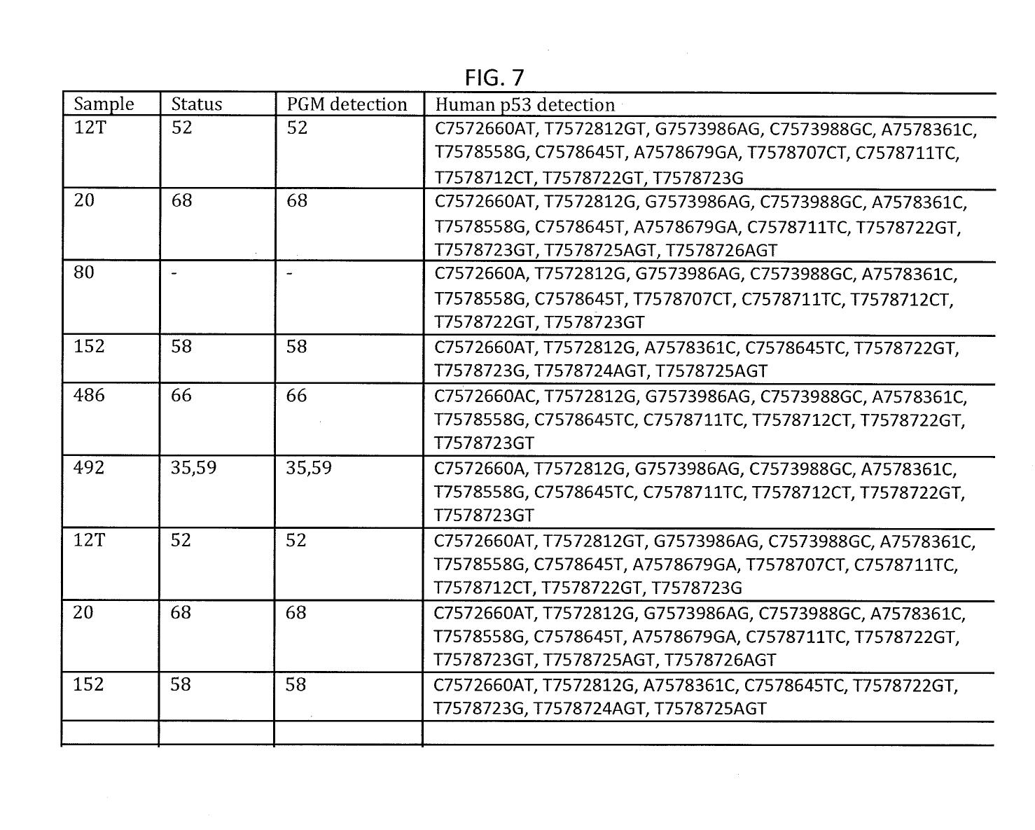

FIG. 8 DNA from Nine Thinprep cervical brush samples were assayed using three techniques: Roche HPV Linear Array kit, Cervista Invader technology, and a molecular inversion probeset (Dx-seq) containing probes targeting 30 high-risk HPV variants. The Roche and Cervista assays were performed as to manufacturer's instructions, and the molecular inversion probeset was sequenced on the Ion Torrent PGM platform. The results for HPV subtype identification are recorded and compared between technologies. The results demonstrate cases in which the Roche and or Cervista technology are unable to determine the HPV subtype present with a sample, but Dx-seq identifies a HPV subtype present, and also cases in which discordance between Roche and Cervista tests is resolved by the Dx-seq test, which confirms the subtype present within the sample. Also illustrated is an example in which the Dx-seq tests detects multiple HPV strains present within a sample, a case in which neither competing technology can accurately determine that both subtypes are present within the sample. The final column of the table demonstrates the ability to stratify specific HPV type by previously assessed risk criteria, e.g. established pathoglogical standard practice. Infections are classified by the type of condition most associated with (e.g. genital warts), or the calculated risk of developing cervical cancer.

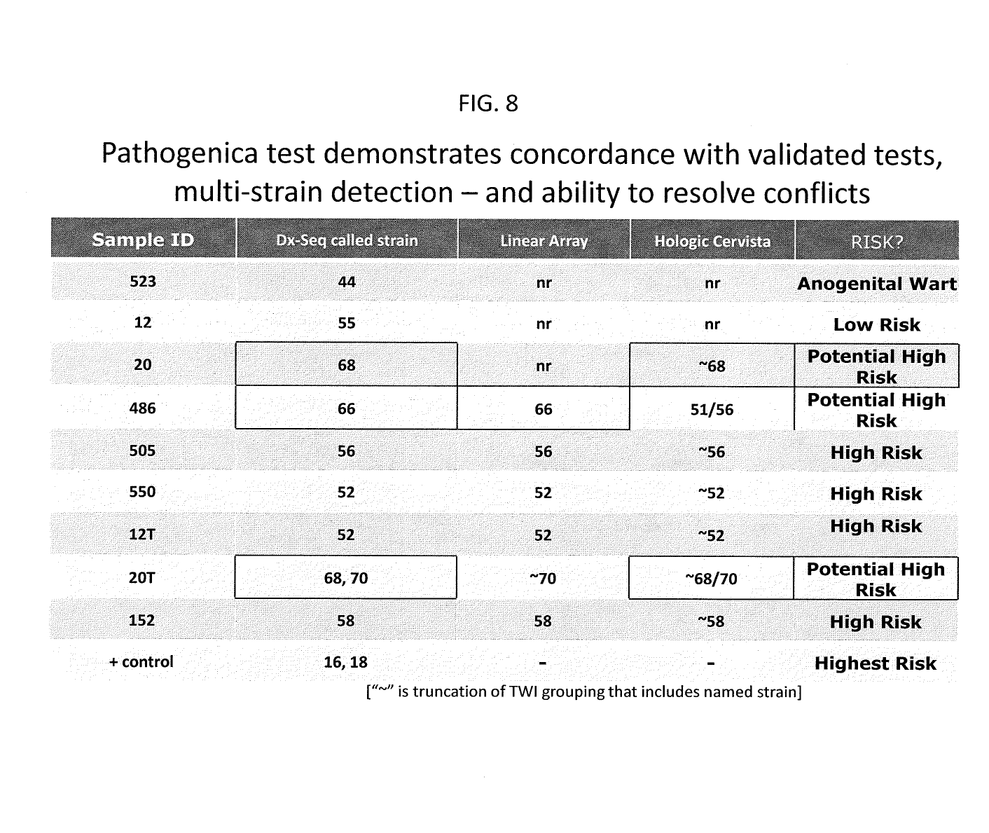

FIG. 9 DNA from Thinprep cervical brush samples YP1, YP10, YP 26, YP26, YP28 was assayed using a molecular inversion probeset containing probes targeting 30 high-risk HPV variants. Additionally, the probeset included probes capable of circularizing on Lactobacillus and Candida genomic DNA. Sample YP1 was sub-aliquoted, and genomic DNA from Candida albicans added to create a “spiked sample”. Sequencing libraries were prepared and sequenced on the Ion Torrent PGM. The table indicates the HPV subtype detected from each sample, and additional Lactobacillus or Candida genomic DNA detected in each sample (relative proportions in brackets), demonstrating the correct detection of both HPV viral and bacterial or fungal DNA from a Thinprep sample. The bar graph further illustrates reproducible quantitative detection between replicates of YP1 sample.

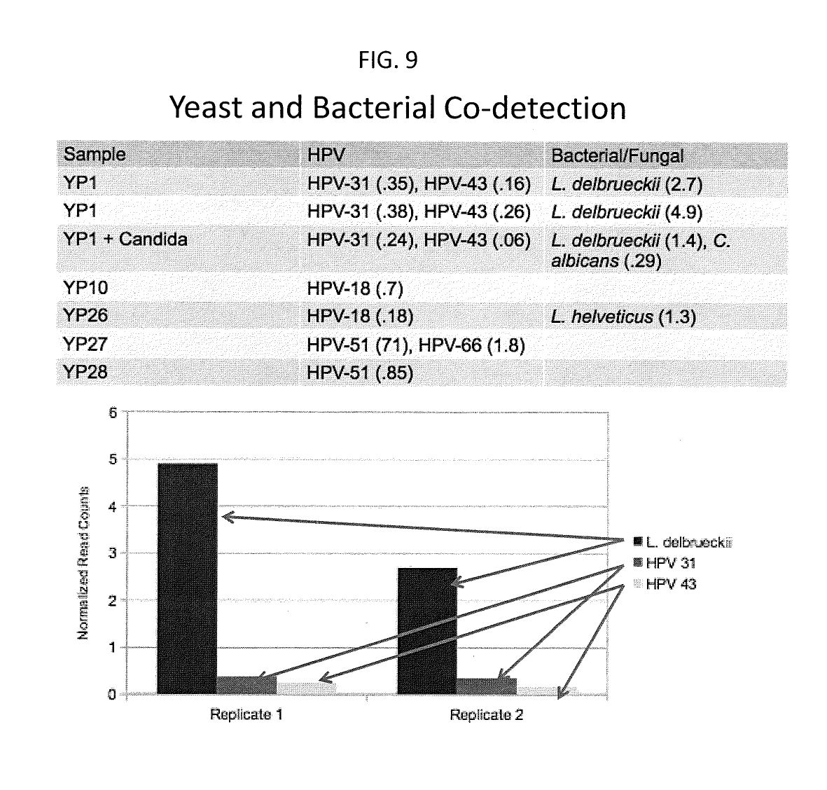

FIG. 10 Viral genomic DNA from HPV 16 was quantified, and added to human genomic DNA samples in copy numbers from 1000 to 10000000. These samples were assayed using a molecular inversion probeset containing probes targeting 30 high-risk HPV variants, and an internal calibration control sequence. Libraries were prepared and sequenced on an Ion Torrent PGM. The readcounts aligning to HPV 16 genomic sequence were quantified and normalized using the internal calibration control. A tight linear correlation between input copy number and sequencing read quantification is demonstrated.

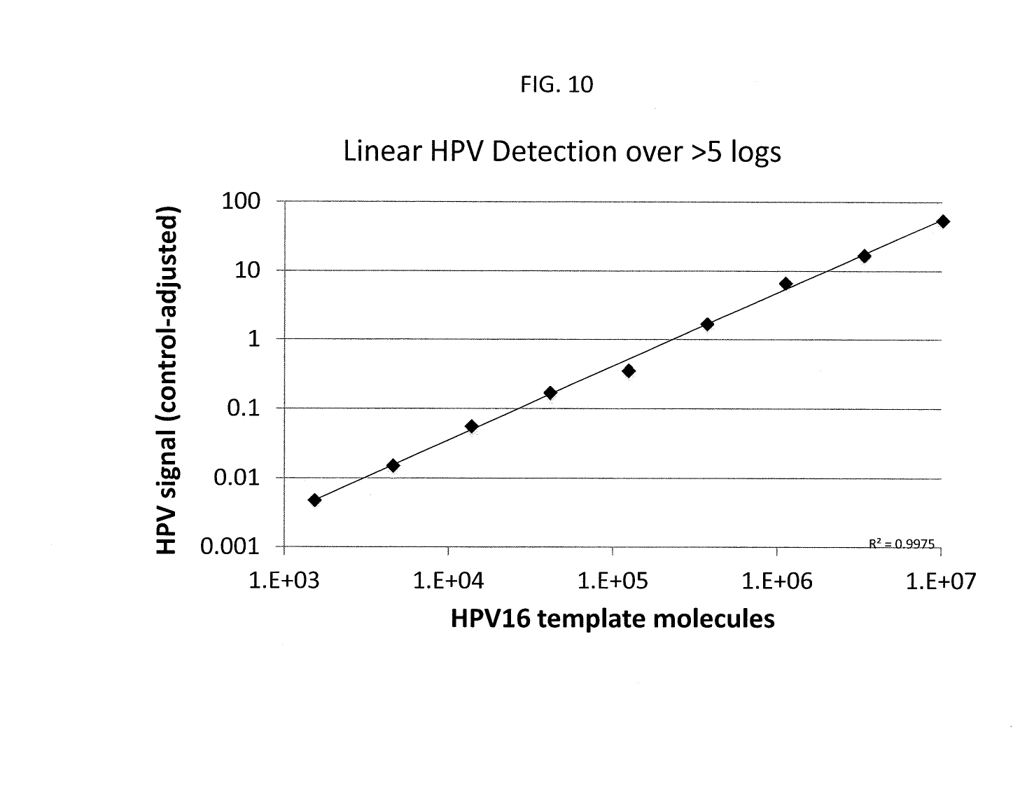

FIG. 11 Viral genomic cDNA from HIV CNO09 was quantified, and added to human genomic DNA samples in copy numbers from 10 to 100000000. These samples were assayed using a molecular inversion probeset containing probes targeting resistance gene regions within the HIV genome. Libraries were prepared and sequenced on an Ion Torrent PGM. The readcounts aligning to HIV genomic sequence were quantified. A tight linear correlation between input copy number and sequencing read quantification is demonstrated over 6 orders of magnitude.

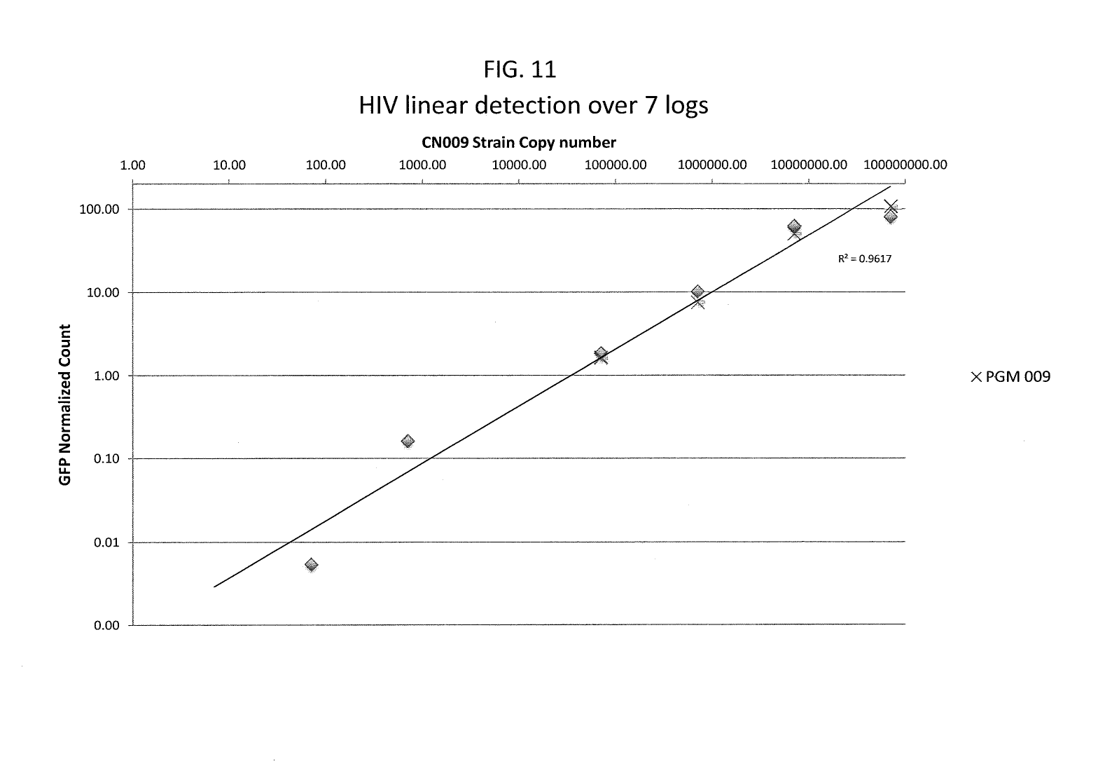

FIG. 12 Four genomic DNA samples from Enterococcus bacteria were sequenced using a multiplex probeset of >400 molecular inversion probes designed to capture >12 common bacterial pathogens. Libraries were sequenced on an Ion Torrent PGM. Sequence reads from a subset of these probes were aligned to the expected reads from Enterococcus genomes, and concatenated into a contig representing the Enterococcus genotype for this probeset. An alignment of a fraction of this contig that varies between the four samples is illustrated, which demonstrates >30 nucleotide differences that enable the four samples to be distinguished from each other with >99% specificity (taking into account the error characteristics of this sequencing platform, these specific probes, and the variance within the Enterococcus genome).

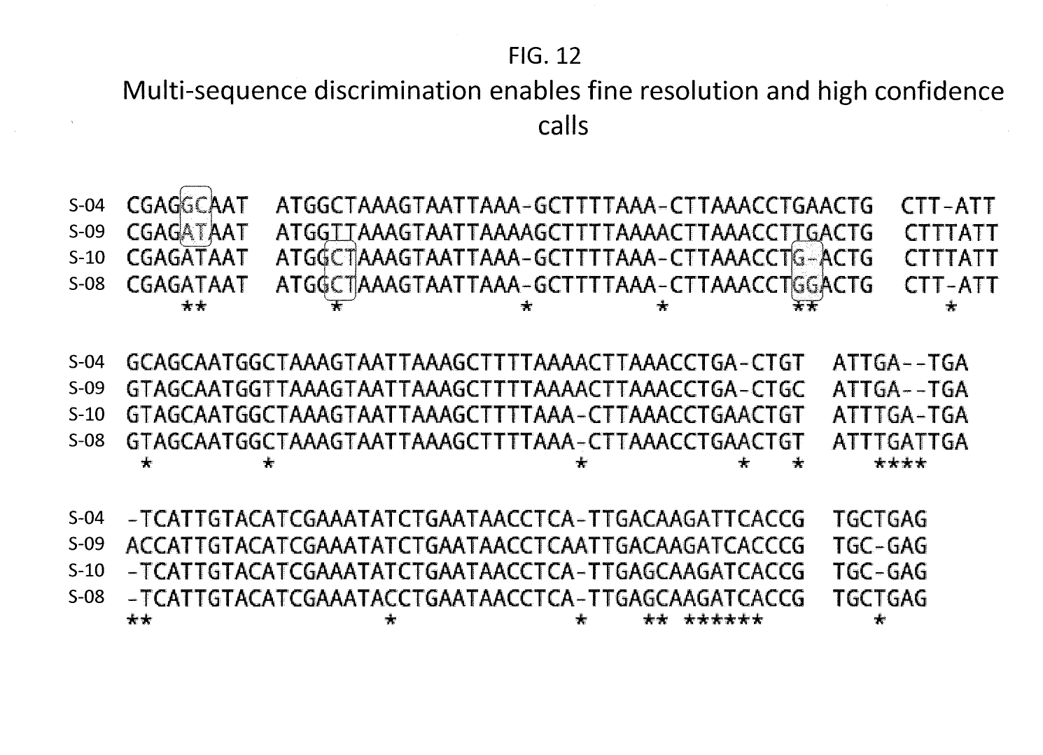

FIG. 13 Five synthetic 100 base DNA constructs were synthesized, each containing common “5′ Synthetic Gene Regions” and “3′ Synthetic Gene Regions”, but differing by a central “Synthetic Gene Variable Region” of 6 nucleotides. The synthetic sequences indicated WT Control, 1 and 2 were mixed into a sample, and contacted by a molecular inversion probeset designed to bind to ˜25 nucleotide regions of the 5′ 3′ synthetic gene regions. Libraries were sequenced on an Ion Torrent PGM, and the readcount for each synthetic construct quantified, revealing high readcount detection of WT control, and synthetic sequences 1 and 2. Sequence 3 was correctly absent, whereas sequences 4 and 5 produced low readcounts attributed to background contamination and sequence errors.

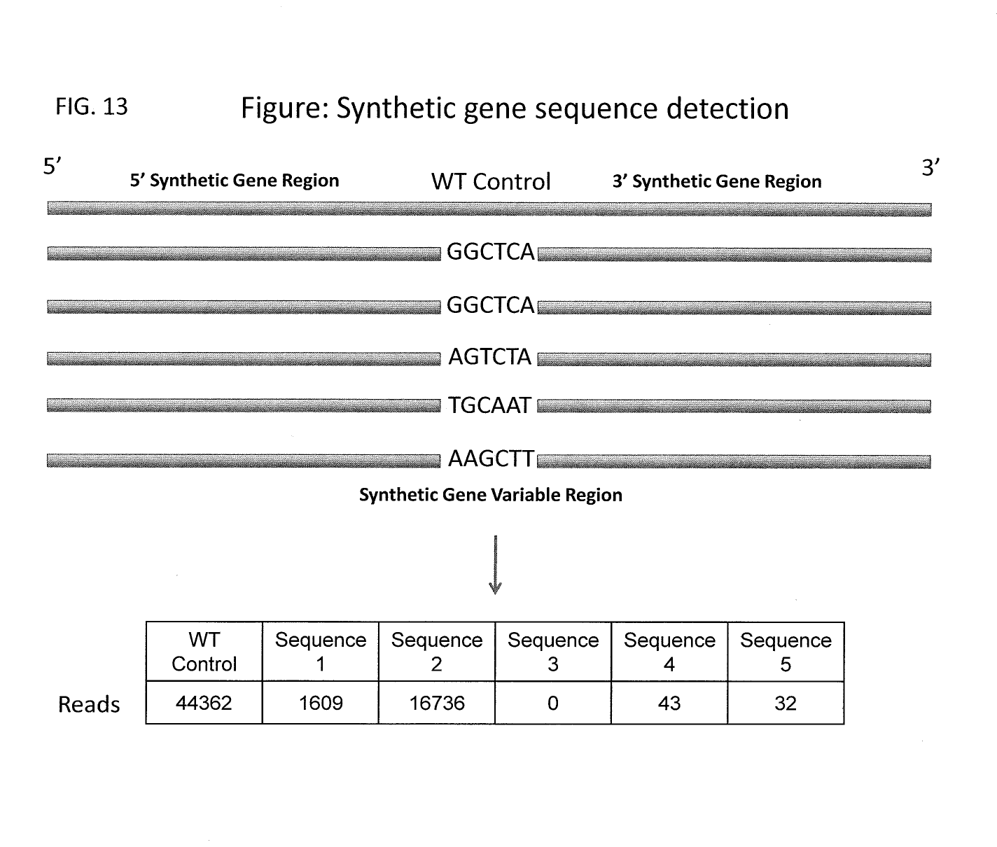

FIG. 14 A molecular inversion probeset was contacted with a control target sequence, and subjected to varying DX-seq assay conditions in terms of amplification primer content, library dilution and amplification stage cycle number. DNA products produced were visualized on a 1% agarose gel using Sybr Safe stain. The resultant amplification products demonstrate controlled production of concatemer sequences of defined unit length that were further verified by Sanger sequencing, and long unit spanning reads generated from Ion Torrent PGM library sequencing.

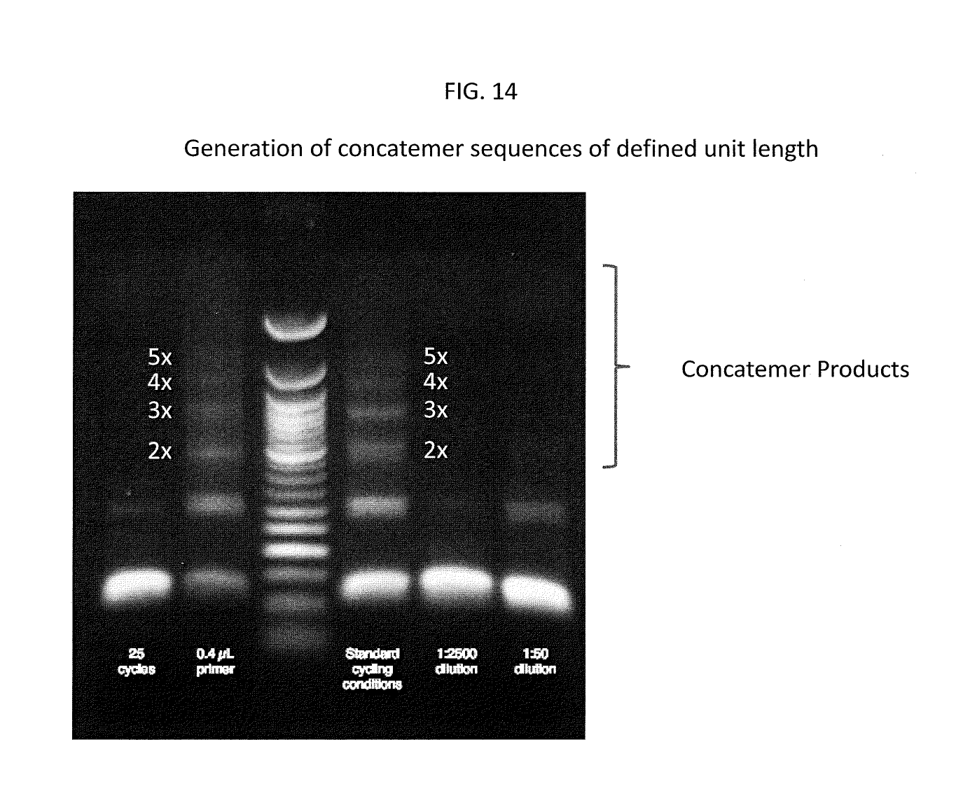

FIG. 15 Biotinylated synthetic dsDNA sequences were prepared. The DNA comprised known sequence flanking variable barcode sequences (labeled “GFP-WT” and “GFP-A”). The synthetic DNA sequences were separately bound via their biotin moiety to a steptavidin-antibody conjugate with high affinity for Green fluorescent protein (GFP). This generated antibody-DNA fusions that differed by their attached DNA sequence. Each antibody-DNA fusion was incubated separately with a GFP-HisTag protein, washed with binding buffer, and precipitated using magnetic bead conjugated antibody that binds to the HisTag portion of the GFP protein. Precipitated antibody-protein-DNA mixture was subject to a molecular inversion probe assay specific to the known flanking sequences of the synthetic DNA. Following PCR amplification the products were visualized on a 1% agarose gel using Sybr Safe stain, and indicated the precipitation of antibody-DNA sequence by the HisTag magnetic beads (lanes 5,6,7). A small amount of synthetic DNA was detected in the sample with no precipitating beads (lane 3), which may be due to insufficient washing of the sample tubes, but precipitation resulted in a 5-10 fold greater recovery of synthetic DNA. These results are taken to demonstrate the ability of a DNA-antibody conjugate to bind to a target protein and be detected by a molecular inversion probe assay in preparation for next generation sequencing.

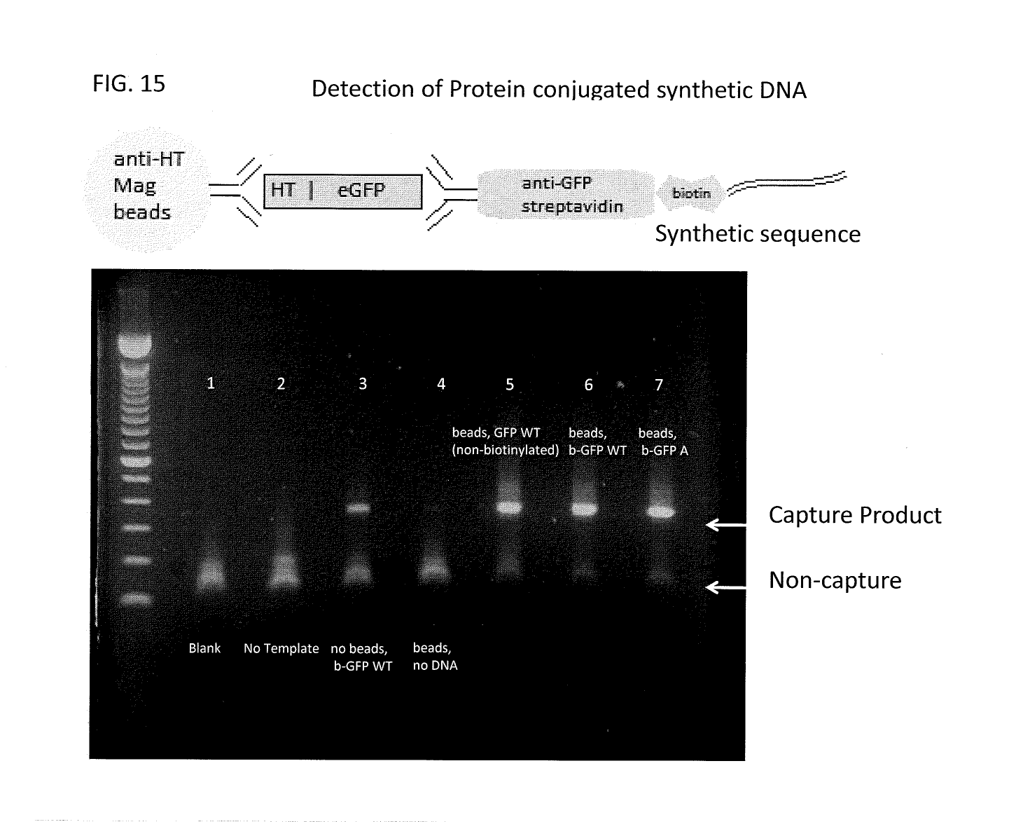

FIG. 16 A molecular inversion probeset designed to detect 13 common bacterial pathogens was used to assay pure genomic DNA isolated from each of the 13 pathogens, and the resulting sequencing libraries sequenced on the Ion Torrent PGM. Each genomic DNA sample was assayed in triplicate at 3 different copy number amounts in the molecular inversion probe assay. The results were analyzed using a 30 minute automated bioinformatics plugin specific for this probeset. Pass criteria indicated detection of >1000 reads of the target pathogen, with less than 100 reads of an unexpected pathogen from the pure gDNA samples. User errors were identified in cases of manual error or sample mix-ups, or failure was indicated if the sample did not meet the pass criteria. The table indicates that of 139 samples tested, there were 9 cases of user error, and only one case of assay failure. There were no cases in which the sample pathogens were misidentified as another species. This indicates a >99% sensitivity and specificity for this assay.

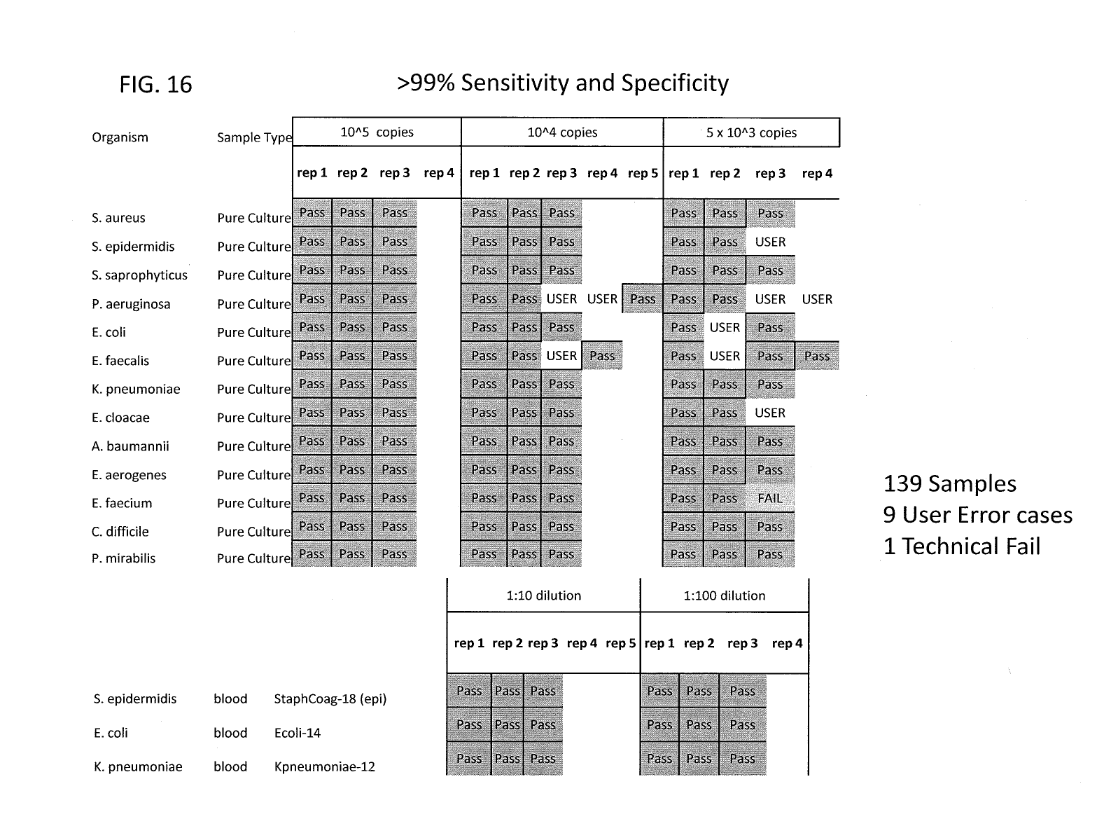

FIG. 17 A protocol is described in which a molecular inversion probe assay is performed by serial addition of components to a single ependorf tube during a 2 hr 35 minute protocol within a thermal cycler. This protocol enables the detection of target nucleic acid within a sample, and preparation of a DNA library for sequencing on an Ion Torrent PGM, but is compatible with other next generation sequencing technologies.

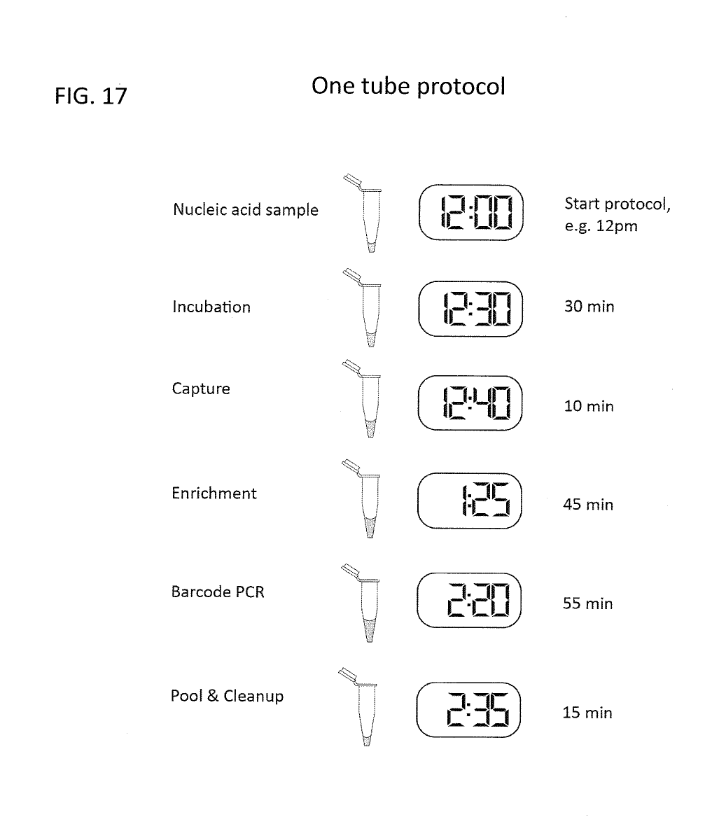

### DETAILED DESCRIPTION

### Definitions

“Capture primers” are linear oligonucleotides suitable for use in methods of polymerase and/or ligase-mediated capture of a region of interest. Capture primers can be either a “conventional” pair of linear oligonucleotide primers with their 3′ ends oriented towards each other suitable for polymerase chain reaction amplification of an intervening region (the “region of interest”) between the regions bound by the pair or a “circularizing capture primer,” also known a molecular inversion probe (MIP), which is a single linear oligonucleotide comprising two homologous probe regions that hybridize to nucleic acid regions adjacent to the region of interest and is suitable for polymerase and/or ligase-mediated circularizing capture of the region of interest.

A “panel” of capture primers is a plurality of capture primers, e.g., either two or more pairs of “conventional” primers or two or more “circularizing capture primers” directed to one or more predetermined organisms of interest.

“High specificity” refers to at least 80% specificity, e.g., at least 80, 85, 86, 86, 88, 89, 90, 91, 92, 93, 94, 95, 95,5, 96, 96.5, 97, 97.5, 98, 98.5, 99, 99.1, 99.2, 99.3, 99.4, 99.5, 99.6, 99.7, 99.8, 99.9, 99.95, 99.99, 99.995, 99.999%, or more, specificity.

“Specificity” as used in this application is the fraction or percent of cases in which the organism is correctly identified when the test detects an organism. “Sensitivity” is one minus the fraction (or 100 minus the percent) of cases in which the test returns “no organism present” when an organism was present in the sample. The methods provided by the invention provide panels of capture primers that achieve at least 80, 85, 86, 86, 88, 89, 90, 91, 92, 93, 94, 95, 95,5, 96, 96.5, 97, 97.5, 98, 98.5, 99, 99.1, 99.2, 99.3, 99.4, 99.5, 99.6, 99.7, 99.8, 99.9, 99.95, 99.99, 99.995, 99.999%, or more, sensitivity.

“Error probability of nucleic acid sequencing” is an error function for sequencing results that accounts for the nucleic acid sequencing modality and organism(s) being sequenced.

“Multiplex organism detection” refers to method of simultaneously detecting and resolving the presence of two or more organisms that may be present in a sample.

“Sequencing library” refers to a collection of nucleic acids suitable for sequencing, either directly without further amplification, with additional amplification, and/or by appending additional nucleic acid sequences, such as adapters for a particular sequencing modality. In certain embodiments, a sequencing library is suitable for nucleic acid sequencing in the absence of additional nucleic acid amplification. In other embodiments, the sequencing library may undergo addition amplification. In more particular embodiments of methods either entailing additional amplification or not, additional sequences can be appended to the termini of the nucleic acids to be sequences, e.g., adapter sequences suitable for use in a particular sequencing modality. In certain embodiments, adapter sequences are appended to the sequencing library in the amplification step.

“Circularizing capture” refers to a circularizing capture primer becoming circularized by incorporating the sequence complementary to a region of interest. Basic design principles for circularizing capture primers, such as simple molecular inversion probes (MIPs) as well as related capture probes are known in the art and described in, for example, Nilsson et al., Science, 265:2085-88 (1994), Hardenbol et al., Genome Res., 15:269-75 (2005), Akharas et al., PLOS One, 9:e915 (2007), Porecca et al., Nature Methods, 4:931-36 (2007); Deng et al., Nat. Biotechnol., 27(4):353-60 (2009), U.S. Pat. Nos. 7,700,323 and 6,858,412, and International Publications WO 2011/156795, WO/1999/049079 and WO/1995/022623.

Certain aspects of the invention encompass a circularizing capture primer comprising a nucleic acid sequence of the formula:

5′-A-B-C-3′

wherein
A is a probe arm sequence listed in column 1 of table 1 or 3; and
C is the corresponding probe arm sequence listed in column 2 of table 1 or 3 and
B is a backbone sequence.

A circularizing capture primer may further comprise a backbone sequence, which contains a primer binding site between the homologous probe sequences. Typically, the homologous probe sequence at the 3′ end of the circularizing capture primer (probe segment C) is termed the extension arm and the homologous probe sequence at the 5′ end of the circularizing capture primer (probe segment A) is termed the ligation or anchor arm. Upon hybridization to the target sites in the genome of interest, the circularizing capture primer/target duplexes are suitable substrates for polymerase-dependent incorporation of at least two nucleotides on the probe (on the extension arm), and/or ligase-dependent circularization of the circularizing capture primer (either by circularizing a polymerase-extended circularizing capture primer or by sequence-dependent ligation of a linking polynucleotide that spans the region of interest).

“Capture reaction” refers to a process where one or more circularizing capture primers are contacted with a test sample has possibly undergone circularizing capture of a region of interest, wherein the first and second homologous probe sequences in the circularizing capture primer have specifically hybridized to their respective target sequence in the test sample to capture the region of interest between the first and second target sequences of the circularizing capture primer. A capture reaction may produce no circularized products containing a region of interest if none of the organisms targeted by the circularizing capture primers were present in the sample. “Capture reaction products” refers to the mixture of nucleic acids produced by completing a capture reaction with a test sample. “Amplification reaction” refers to the process of amplifying capture reaction products. An “amplification reaction product” refers to the mixture of nucleic acids produced by completing an amplification reaction with a capture reaction product.

A “homologous probe sequence” is a portion of a circularizing capture primer provided by the invention that specifically hybridizes to a target sequence present in the genome of a target organism. The terms “homologous probe sequence,” “probe arm,” “homologous probe arm,” “homer,” and “probe homology region” each refer to homologous probe sequences that may specifically hybridize to target genomic sequences, and are used interchangeably herein. “Target sequence” refers to a nucleic acid sequence on a single strand of nucleic acid in the genome of an organism of interest. In some embodiments, the homologous probe sequences in the circularizing capture primerare the sequences listed in tables 1 or 3, or their reverse complement. The term “hybridizes” refers to sequence-specific interactions between nucleic acids by Watson-Crick base-pairing (A with T or U and G with C). “Specifically hybridizes” means a nucleic acid hybridizes to a target sequence with a Tₘ of not more than 14° C. below that of a perfect complement to the target sequence.

An “organism” is any biologic with a genome, including viruses, bacteria, archaea, and eukaryotes including plantae, fungi, protists, and animals.

“Region of interest” refers to the sequence between the nearest termini of the two target sequences of the homologous probe sequences in a capture primer (i.e. a conventional primer pair or circularizing capture primer.

The capture primers provided by the invention may comprise the naturally occurring conventional nucleotides A, C, G, T, and U (in deoxyriobose and/or ribose forms) as well as modified nucleotides such as 2′O-Methyl-modified nucleotides (Dunlap et al, Biochemistry. 10(13):2581-7 (1971)), artificial base pairs such as IsodC or IsodG, or abasic furans (such as dSpacer) (Chakravorty, et al. Methods Mol Biol. 634:175-85 (2010)), that do not form canonical Watson-Crick hydrogen bonds), biotinylated nucleotides, adenylated nucleotides, nucleotides comprising blocking groups (including photocleavable blocking groups), and locked nucleic acids (LNAs; modified ribonucleotides, which provide enhanced base stacking interactions in a polynucleic acid; see, e.g., Levin et al. Nucleic Acid Res. 34(20):142 (2006)), as well as a peptide nucleic acid backbone. In particular embodiments, the 5′ or 3′ homologous probe sequences of a capture primer provided by the invention comprise, at their respective termini, a photocleavable blocking group, such as PC-biotin. In more particular embodiments, a capture primer provided by the invention comprises a photocleavable blocking group at its 5′ terminus to block ligation until photoactivation. In other particular embodiments, a capture primer provided by the invention comprises at its 3′ terminus a photocleavable blocking group to block polymerase-dependent extension or n-mer oligonucleotide ligation until photoactivation.

In other embodiments, the 5′-most nucleotide of a capture primer provided by the invention comprises an adenylated nucleotide to improve ligation and/or hybridization efficiency. See, e.g., Hogrefe et al., J Biol. Chem. 265 (10): 5561-5566, (1990). In more particular embodiments, the 5′ end of the 5′ homologous probe region (e.g., the ligation arm) comprises at least one LNA and in still more particular embodiments, the 5′ terminal nucleotide is a LNA.

In a particular embodiment, the capture primers are capped with a phosphate group at the 5′ end to improve the ligation efficiency.

The term “barcode” is used to refer to a nucleotide sequence that uniquely identifies a molecule or class of related molecules. Suitable barcode sequences that may be used in the capture primer s of the invention may include, for example, sequences corresponding to customized or prefabricated nucleic acid arrays, such as n-mer arrays as described in U.S. Pat. No. 5,445,934 to Fodor et al. and U.S. Pat. No. 5,635,400 to Brenner. In certain embodiments, the n-mer barcode may be at least 3, 4, 5, 6, 7, 8, 9, 10, 12, 15, 16, 17, 18, 19, 20, 21, 22, 23, 24, 25, 30, 35, 40, 50, 60, 70, 80, 90, 100, 200, 300, 400 or 500 nucleotides, e.g., from 18 to 20, 21, 22, 23, 24, or 25 nucleotides. In particular embodiments, the n-mer barcode is from 6 to 8 nucleotides. In further embodiments, the n-mer barcode is from 10 to 12 nucleotides. In particular embodiments the barcodes include sequences that have been designed to require greater than 1, 2, 3, 4 or 5 sequencing errors to allow this barcode to be inadvertently read as another in error. In some embodiments, the capture primers do not contain a barcode, while a primer that is used to amplify a circularized capture primer contains a barcode.

Selection of barcodes that may be utilized in a panel of capture primers used to test a sample from a patient may involve selecting a combination of barcodes that will provide >5% and not more than 50% representation of a particular nucleotide at each position in the barcode sequence within the pool. This is achieved by random addition and removal of barcodes to a pooled set until the conditions specified are met using a Perl script. Barcodes for which the reverse complement sequence is also present within the barcode pool may also be eliminated.

In some embodiments, the barcode is sample-specific, e.g., comprises one or more patient specific barcodes. In particular embodiments, more than one barcode will be assigned per patient sample, allowing replicate samples for each patient to be performed within the same sequencing reaction. By using sample nucleic acid-specific barcodes it is possible to both multiplex reactions as described in the present application, as well as detect cross-contamination between test samples that did not use a defined repertoire of specific barcodes. In certain embodiments, the barcode may be temporal, e.g., a barcode that specifies a particular period of time. By using a temporal barcode, it is possible to detect carry-over or contamination on an assay instrument, such as a sequencing instrument, between runs on different days. In more specific embodiments, sample and/or temporal barcodes may be used to automatically detect cross-contamination between samples and/or days and, for example, instruct an instrument operator to clean and/or decontaminate a sample handling system, such as a sequencing instrument.

In certain embodiments, the mixtures of the invention contain sample internal calibration nucleic acids (SICs). In particular embodiments, known quantities of one or more SICs are included in a mixture provided by the invention. In particular embodiments, at least 1, 2, 3, 4, 5, 6, 7, 8, 10, 15, 20, 25, or 30 different SICs are included in the mixture. In particular embodiments, there are about 4 different SICs in a mixture. In some embodiments, the SICs have a nucleotide composition characteristic of pathogenic DNA targets and are present in specific molar quantities that allow for reconstruction of a calibration curve for quality control, e.g., for the processing and sequencing steps for each individual test sample. In certain embodiments, the SICs makes up approximately 10% (molar quantity) of nucleic acids in a mixture, for example, 2, 4, 6, 8, 10, 12, 14, 16, 18, or 20% (molar) of nucleic acids in the mixture. In particular embodiments different SICs are present in different concentrations, for example, in a dilution series, over a 1, 2, 3, 4, 5, 6, 7, 8, 9, 10, 20, 50, 100, 200, 500, 1000, 5000, 10000, 50000, or 100000-fold concentration range from the most dilute to most concentrated SICs in 2, 3, 4, 5, 6, 7, 8, 9, 10, 20, 30, 40, or 50 steps. In particular embodiments, SICs are present in a sample (e.g., a mixture of capture primers and a test sample, a capture reaction, a capture reaction product, an amplification reaction, or an amplification reaction product) at concentrations of 5, 25, 100, and 250 copies/ml. By detecting the predetermined concentration of the SICs—for example, by using capture primers directed to the SICs—the skilled artisan can estimate the concentration of an organism of interest such as a virus in a test sample. In certain embodiments, this is accomplished by correlating the frequency that a captured sequence is detected to the volume of the sample from which the nucleic acids were obtained. Thus, an organism count per unit volume (e.g., copies/mL for liquid samples such as blood or urine) can be estimated for each organism detected.

In particular embodiments, the concentration of SICs and capture primers directed to the SICs are adjusted empirically so that sequences of SICs detected in a capture reaction product and/or amplification reaction product make up about 2, 4, 6, 8, 10, 12, 14, 16, 18, 20, 25, or 30% of sequences in the mixture. In particular embodiments, SICs make up 10-20% of sequence reads. In certain embodiments, the number of SICs sequence reads in a sequencing reaction is quantitatively evaluated to ensure that sample processing occurs within pre-defined parameters. In particular embodiments, the pre-defined parameters include one or more of the following: reproducibility within two standard deviations relative to all samples sequenced during a particular run, empirically determined criteria for reliable sequencing data (e.g., base calling reliability, error scores, percentage composition of total sequencing reads for each capture primer per target organism), no greater than about 15% deviation of GC or AU-rich SICs within a sequencing run. In embodiments in which patient samples are barcoded to allow pooling for multiplex sequencing, the SICs DNA in a sample will also comprise the same barcode(s) corresponding to unique samples, e.g., particular patient samples.

Test samples may be from any source and include swabs or extracts of any surface, or biological samples, such as patient samples.

Patients may be of any age, including adults, adolescents, and infants.

Biological samples from a subject or patient may include blood, whole cells, tissues, or organs, or biopsies comprising tissues originating from any of the three primordial germ layers—ectoderm, mesoderm or endoderm. Exemplary cell or tissue sources include skin, heart, skeletal muscle, smooth muscle, kidney, liver, lungs, bone, pancreas, central nervous tissue, peripheral nervous tissue, circulatory tissue, lymphoid tissue, intestine, spleen, thyroid, connective tissue, or gonad. Test samples may be obtained and immediately assayed or, alternatively processed by mixing, chemical treatment, fixation/preservation, freezing, or culturing. Biological samples from a subject include blood, pleural fluid, milk, colostrums, lymph, serum, plasma, urine, cerebrospinal fluid, synovial fluid, saliva, semen, tears, and feces. In particular embodiments, the biological sample is blood. Other samples include swabs, washes, lavages, discharges, or aspirates (such as, nasal, oral, nasopharyngeal, oropharyngeal, esophagal, gastric, rectal, or vaginal, swabs, washes, ravages, discharges, or aspirates), and combinations thereof, including combinations with any of the preceding biopsy materials.

### Capture Primers for Use in Methods Provided by the Invention

The methods provided by the invention employ capture primers as defined herein and described more fully in International Publication WO 2011/156795, which is incorporated by reference in its entirety (encompassing both the descriptions of conventional primer pair and molecular inversion probes (MIPs)).

### Selecting Regions to Sequence to Achieve Specificity

A number of inventions allow for the design of primers or probes to enable the selective sequencing or enrichment of a set of pieces of DNA from a complex sample of DNA molecules. For example, Life Technologies offers the Ion AmpliSeg™ Designer to design primer pairs for use in a multiplex PCR reaction. Similarly, Agilent offers custom panels for its SureSelect and HaloPlex products in which a customer can submit sequences to be captured. When using these techniques to design primers or probes to identify species or strains, the designer must choose a level of redudancy-how many SNPs or other differences should distinguish every pair of species or strains? Fewer probes or primers reduces the cost of the assay but may be more prone to erroneous results.

The present invention allows one skilled in the art to use any method of picking primers or probes that reveal differences between genomes to achieve a desired specificity in the face of potential sources of error in the experiment:
1. Sequencing error. All DNA sequencing technologies make mistakes with some frequency. Sequencing machines and the accompanying data analysis software typically achieve error rates around 1%.
2. Natural genomic variability. A method that distinguishes two species on the basis of a single nucleotide will report incorrect results with a frequency dependent on the natural frequency with which that nucleotide varies within isolates of a species.

A simple solution to these problems is to sequence more nucleotides. However, sequencing more of a genome incurs greater cost, as does increasing the number of regions sequenced by a probe set. Thus, it is advantageous to sequence the smallest number of regions, or an approximation thereof, that achieve the desired specificity. Note that the use of “probe” in the description of this invention is not limited to any particular type of probe; any invention able to select particular DNA molecules from a mixture may be used, including molecular inversion probes, microarray capture probes, bead-based capture probes, or primer pairs.

The present invention provides a method for using a probe selector or probe set designer to achieve a desired specificity. This invention uses estimates of the two error rates, p_error_seq and p_error_genome, to determine the number of differences that the probe set will sequence. These error rates may be summed into a single p_error that indicates the probability of an unreliable or incorrect observation at any nucleotide in the regions sequenced. The sequencing can be by second generation or third generation sequencing methods, such as using commercial platforms such as Illumina, 454, Solid, Ion Torrent, PacBio, Oxford, Life Technologies QDot, or any other available sequencing platform.

Consider a probe set that allows the sequencing of some number of genomic regions that are expected to reveal at least N differences between any pair of strains or any pair of species. When analyzing the data, a software tool or a human will decide whether the sample contained organism A or organism B based on a set of at least N informative nucleotides (the informative nucleotides may vary for different pairs of organisms). Knowing that the sequencing data may contain errors or that the isolate may not be perfectly isogenic to A or B, the data interpreter will assign the sample to whichever of A or B is most similar to the sample in the regions sequenced. Thus, if the sample contains A, the interpreter will assign the sample to A if the sequencing data matches A at a majority of the N or more informative nucleotides. Likewise, the interpreter will assign the sample to B if the sequencing data matches B at a majority of the N or more informative nucleotides. Thus, given the N informative nucleotides, the interpreter will make the correct decision if at least floor(N/2)+1 of the nucleotides are “correct” in that they were sequenced correctly and they have not mutated in the isolate in the sample relative to the correct reference strain.

To design the probe set such that the interpreter will make the correct assignment between A and B at least 99% of the time (that is, 99% specificity distinguishing A from B), the number of informative nucleotides N must be large enough that the probability that a majority are wrong is less than 99% given the sources of error. This process can be modeled with the Binomial distribution. More specifically, the probability of an incorrect assignment is described by Formula 1, below, the cumulative distribution function of the Binomial Distribution where N is the number of informative loci and p is the probability of an incorrect nucleotide observation (p=p_error_seq+p_error_genome):

$${\sum }_{X = ⌊ N / 2 ⌋ + 1}^{N} ( \begin{matrix}N \\ X\end{matrix} ) \cdot {p}^{X} \cdot {( 1 - p )}^{N - X}$$

For example, given 10 informative loci and a probability of error of 0.1, the probability that the interpreter makes an incorrect assignment is 1.5×10̂−4. Using the same 10 loci, the error probability could be as high as 0.22 without decreasing the specificity below 99%. The table below gives the probability of error for various values of N (the number of informative loci) and the error probability:

| | per-base error | |
|---|---|---|
| N | probability | specificity |
| 3 | .1 | .972 |
| 5 | .1 | .991 |
| 10 | .1 | >.999 |
| 20 | .1 | >.999 |
| 3 | .25 | .896 |
| 5 | .25 | .942 |
| 10 | .25 | .994 |
| 20 | .25 | >.999 |

Given an estimate of the combined error probabilities and a desired specificity, a value for N can be determined by a variety of methods, for example:
1. set N=1
2. using equation 1, compute the probability of an incorrect assignment
3. if the desired specificity is greater than one minus the probability of an incorrect assignment, increment N and go to step 2. Otherwise, stop.

This procedure can be implemented in many common scientific or statistical tools such as R, Matlab, Octave, etc.

The above method for determining the number of informative loci needed to achieve a desired specificity relies on the assumption that the informative loci report incorrect results independently of each other. However, this may not be true if several informative loci are nearby in the genome, such as when they are captured by a single probe or primer pair and observed by a single sequencing read. In this case, the set of loci may act as a single unit. For example, the native copy of a gene may be replaced by a foreign version transferred from another strain or species on a plasmid, thus generating multiple differences from a reference genome simultaneously. Thus, a more robust method for choosing informative loci treats sets of proximal loci as a single unit. Rather than letting N represent the number of informative nucleotides, this more conservative approach lets N represent the number of informative probes.

Determining or estimating the two error probabilities is critical for choosing a suitable N. In general, the error characteristics of sequencing machines are well-defined, though they may vary throughout the sequencing read. Numerous software packages such as FastQC, PIQA, and Reptile can plot the quality scores (presented as Q scores, where q=−10*log 10 probability of sequencing error) reported by the sequencing machine. While the quality scores typically decrease over the length of the sequencing read, one can determine a minimum value for a given read length. For example, quality scores from the Ion Torrent PGM are generally above Q20 (p_error_seq<=0.010) 200 nucleotides into the read. A sequencing run of lower quality might yield scores that decrease to Q15, indicating that p_error_seq<=0.031. Thus, a simple approach uses p_error_seq=0.01 for a probeset to be used with an Ion Torrent PGM sequencing machine and 200 bp reads.

Estimating the probability of mutations between a given isolate and a published genome presents a greater challenge. A simple approach uses a known value for the difference between species or between strains. For example, Konstantinidis et al (Phil. Trans. R. Soc. B. 361:1929-40(2006)) suggest that the nucleotide variation between bacterial species is 95% suggesting that p_error_genome=0.05.

The level of divergence or variation may also be computed from a set of sequenced genomes for an organism. For example, the genomes may be aligned using a program such as Muscle, Clustalw, or Mummer and the number of divergence rate computed between each pair of genomes. Then, the average or maximum divergence rate could be used as an estimate for p_error_genome.

A more complicated approach uses a variable value for p_error_genome. The value could be calculated per-base taking into account multiple sequence alignments, boundaries between coding and non-coding regions, a nucleotide's position within a codon, measures of amino acid conservation in a protein family, etc. Use of a variable p_error_genome complicates the task of determining the number of informative nucleotides or probes necessary to achieve a desired specificity as the value of p in equation 1 is no longer constant across all N nucleotides or probes. In fact, the value for p varies depending on which probes are chosen for use in the probe set. Thus, the value for N cannot be calculated before the probe set is chosen. Instead, the probability of an incorrect result is computed as each probe is added to the probe set. This probability of an incorrect result can be computed by summing the probability of X incorrect nucleotides for X=(floor(N/2)+1) to N. If p_error_i is the sum of p_error_seq and p_error_genome at nucleotide I, then the probability of X incorrect nucleotides is the sum, over all configurations of the N nucleotides in which X are incorrect of (the product of p_error_i for I in the X incorrect nucleotides)*(the product of (1−p_error_i) for the remaining nucleotides.

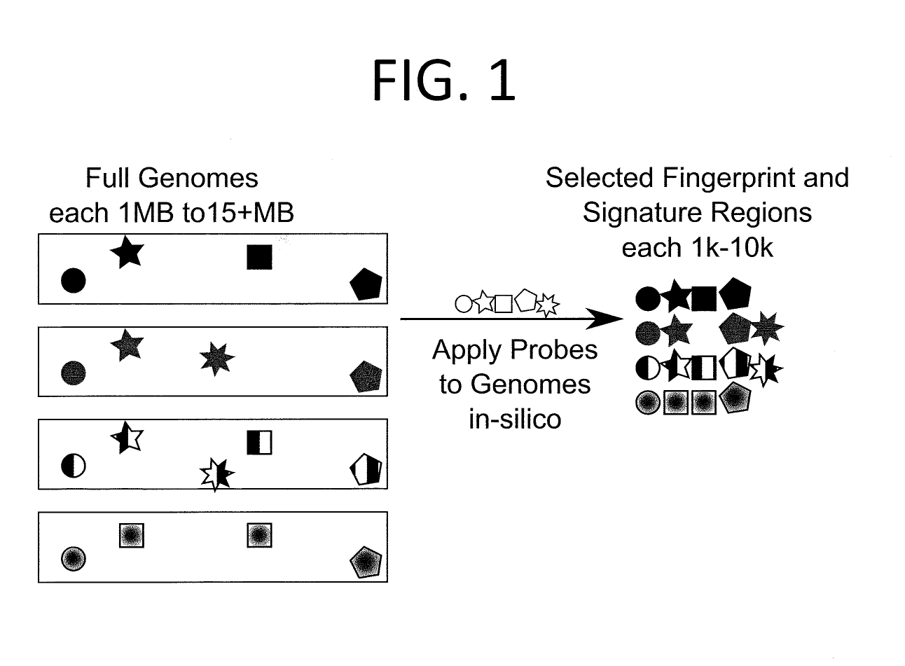

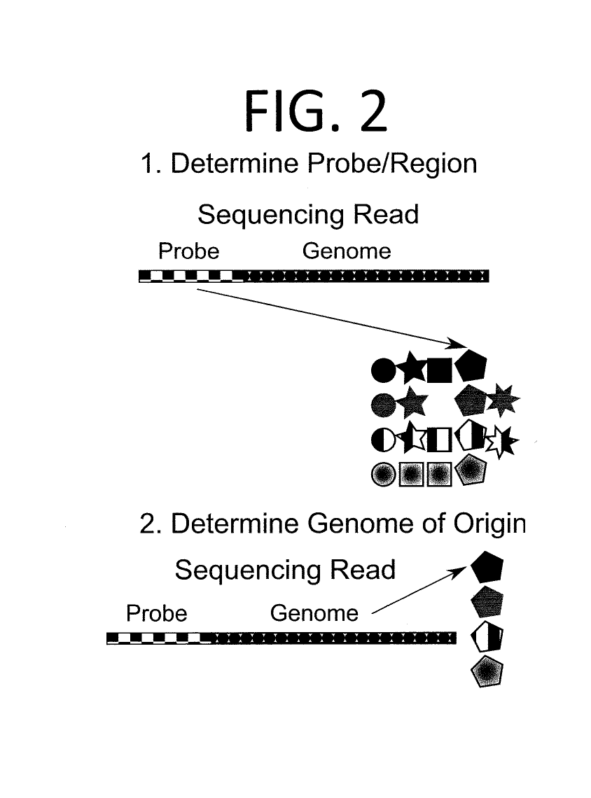

Given the sequencing reads for a set of selected regions, the reads can be analyzed quickly by comparing them to or aligning them to a database that contains the set of reads that could be generated by the probe set applied to a large collection of known full or partial genomes as shown in FIGS. 1 and 2. One skilled in the art can generate this database by aligning the probe sequences against the database of genomes and using the alignments to generate the expected sequencing reads. When using molecular inversion probes or primer pairs, the two ends of the probe or the two primers must map to nearby genomic locations in the correct orientation and will produce an expected read that is the genomic sequence between the two ends. When using hybridization probes, such as Agilent's SureSelect, the single probe sequence is aligned to the database of genomes and matching regions are expanded by a length corresponding to the longest possible read from the sequencing platform to account for the fact that the sequenced DNA fragments will not have well defined boundaries. The set of possible reads from the probe set is then pre-processed according to the aligner that will be used to map the sequencing reads from the sample. For example, common alignment programs such as Blast, Blat, Bowtie, or SOAP all come with a program to process sequences (eg, in a FASTA file) into a database format for the aligner.

This database enables rapid analysis because fraction of any genome selected by the probes is relatively small compared to the size of the genome. For example, a probe set might sequence 5 kb of a Staphylococcus aureus genome, or about 0.1%. Thus, an alignment database that contains the potential results of a probe set applied to thousands of genomes will be only about as large as a database that contained a few full genome sequences. For example, when the probes in Table 3 are applied to a database of hundreds of bacterial and fungal genomes and several mammalian genomes, the resulting alignment database contains only about 3 MB of sequence. Thus, the analysis of the sequencing reads from selected genomic regions relative to hundreds of bacterial genomes takes only as long as would the analysis of those sequencing reads against a single full genome sequence.

### Achieving Specificity by Selecting Regions in the Analysis

In another embodiment, the invention might use a virtual selection rather than a physical selection to analyze the most informative regions of genomes. In this embodiment, standard reagents might be used to generate sequencing reads from the entire genome of the organism or organisms in a sample. Analyzing this data with standard methods, however, is very difficult and requires substantial computing resources. For example, each sequencing read may be aligned against a large collection of genome sequences. Such a database may be dozens or hundreds of gigabases when generated from publicly available sources such as Genbank. As the time required to align reads generally increases linearly with the database size, large databases may become impractical. For example, aligning 10 million reads (as generated by an Illumina MiSeq machine) might take under half an hour to align against the human genome; however, aligning these reads against a database of known bacterial, fungal, and viral, and mammalian genomes might take sixteen hours or more.

Using the methods of probe selection from this disclosure, one skilled in the art can generate a small set of signature or fingerprint regions most useful for identifying a set of organisms. In typical usage, the total size of these regions might be 1/1000th the size of the input genome sequences, thus reducing the read alignment time by a factor of 1000.

When comparing these sequencing reads to the database, the read cannot be split into “probe” and “genome” parts as shown in FIG. 2. Instead, the entire read is “genome” and is compared to a database of genomic regions in a single step. This comparison may be performed using standard programs such as Blast, Blat, Bowtie, Bowtie2, MAQ, etc.

### Synthetic Nucleic Acid and Protein Detection

In addition to detecting nucleic acids from organisms, it is often desirable to detect synthetic nucleic acid sequences, such as from an internal calibration standard, or an exogenously synthesized gene plasmid or product. In some embodiments this synthetic nucleic acid may be associated with or conjugated to a non-nucleic acid biomolecule, or a small molecule, for example biotin, or a protein, for example an antibody. A nucleic acid conjugated to an antibody may be enriched using a secondary molecule with affinity for the antibody, or a molecule to which the antibody is bound with high affinity, such as the target epitope. Determination of the number of antibody molecules enriched may be achieved by sequencing of the synthetic nucleic acid sequence associated with the antibody. In some embodiments this sequencing may be next generation sequencing. In further embodiments the nucleic acid sample may contain a mix of unique synthetic nucleic acid sequences attached to unique antibodies of different identity. In this embodiment, sequencing of this library of synthetic nucleic acids may enable the relative amounts of each antibody present within the mixture to be quantified. In some embodiments this sequencing library is prepared by PCR primers containing a sequence which binds to the synthetic DNA target, and regions that interacts with the sequencing platform of choice. In other embodiments, a molecular inversion probeset may contact the synthetic nucleic acid target and capture the sequence information for next generation sequencing.

As an illustrative example, in a mixture of 10 antibodies in a tube, by preparing each antibody with a separate oligonucleotide conjugated to it, and then mixing the 10 together and then sequencing the abundance of the different sequences, one can then determine how much of each antibody is present in the tube. These methods are useful in a variety of contexts because, for example, antibodies can be contacted with a fixed set of targets, e.g., a tissue sample, and the amount of antibody retained by the tissue sample can subsequently determined by sequencing.

These methods are superior to existing methods, such as detecting the sequences attached by PCR or Sanger sequencing, because the detection method allows detection of individual molecules by the unique sequences attached. Quantifying a mix of 10 or 100 or 1000 labeled biomolecules, such as antibodies in a single tube/sample becomes possible using this aspect of the invention.

### Performing a Sensitive and Specific Selection in a Single Tube

Technologies with simple protocols are advantageous as they allow relatively unskilled technicians to perform the work. Key characteristics of simple protocols are the number of reagents needed, the number of cleanup steps, and the number of transfers from one tube or vessel to another. In many cases, these characteristics also allow for easier automation of a protocol either via microfluidics devices or liquid handling robots.

Several technologies enable the simultaneous capture of many DNA targets from a complex sample: multiplex PCR, molecular inversion probes, hybridization on a surface, or hybridization on beads. However, many of these technologies require complex protocols. For example, the Ampliseq multiplex PCR protocol requires three cleanup/purification steps and the DNA is transferred through five separate tubes. The Nextera library preparation system requires two cleanups and three separate tubes.

The present invention provides a method that allows an unskilled technician can capture hundreds or thousands of genomic regions from a complex sample and prepare them for sequencing using only a single tube per sample and only a single cleanup for an entire batch of samples. This invention uses molecular inversion probes, described in, for example, Nilsson et al., Science, 265:2085-88 (1994), Hardenbol et al., Genome Res., 15:269-75 (2005), Akharas et al., PLOS One, 9:e915 (2007), Porecca et al., Nature Methods, 4:931-36 (2007); Deng et al., Nat. Biotechnol., 27(4):353-60 (2009), U.S. Pat. Nos. 7,700,323 and 6,858,412, and International Publications WO 2011/156795, WO/1999/049079 and WO/1995/022623.

A common limitation of enzymatic nucleic acid amplification is that the mix of components within a reaction can interact to generate unintended products. In the case of detection by gel electrophoresis, a nucleic acid product of defined length may appear to be the predominant species in a sample, but a faint smear of unintentional nucleic acid products of varying sizes may comprise a significant amount of the total nucleic acid product in the reaction. In the case of detection by sequencing, both intended and unintended products may be sequenced, with the latter reducing the proportion of the sequencing reaction that can be usefully interpreted.

Common protocols for preparation of libraries for next generation sequencing include size separation or enrichment steps to reduce the amount of unintended product in a reaction, or transfer of components between multiple ependorf tubes to separate enzymatic steps that interfere with the efficiency of each other. Such steps increase the complexity of a workflow for operators, extend hands on time, and can impede the deployment of such reactions on liquid handling robots, or microfluidic devices. This invention describes an optimized method of sequencing library generation that in which reaction components are added by serial addition into the same volume of sample in the same tube from the steps of contacting the target nucleic acid sample through the completion of library amplification.

In the embodiments described, the nucleic acid target is mixed and incubated with a molecular inversion probe set. To this reaction a high fidelity processive polymerase and a thermostable ligase is then added, mixed and incubated. Further, an exonnuclese activity is added and incubated with the mixture to deplete linear nucleic acids within a sample. Finally, oligonucleotides are added to the mix in the presence of DNA polymerase and a PCR reaction performed to amplify the nucleic acid library within the sample.

The foregoing advantageous methods provided by the invention overcame the production of unwanted products, and requirement for gel electrophoresis of size selection beads prior to library amplification. This was achieved, at least in part by carefully selecting oligonucleotide components that interacted to a minimal extent to produce unwanted products, and employing exonuclease enzymes that eliminated nucleic acids that may be likely to generate unwanted products in the PCR step of library preparation. An exemplary protocol is provided below.

Protocol 1: MIP capture for 14 samples
Prepare the hybridization solution:
   22.5 μL 10× Ampligase buffer
   15 μL probe mix (with each probe at 3 nM)
   37.5 μL Nuclease free water
Add 5 μL of hybridization mix and 10 μL of DNA to each tube. A strip tube or plate with 200 μL wells is ideal.
Begin the MIP program on the thermocycler
   94°, 10 min
   Ramp to 60°, 0.1°/sec
   60°, 10 min
   60° hold
   60°, 10 min
   94° for 2 minutes
   37° hold
   37° for 30 minutes
   94° for 15 minutes
   4° hold
While the hybridization is running, prepare the extension and ligation mix on ice:
   5 μl 2× Phusion High Fidelity PCR Master Mix
   5 μL 10× Ampligase buffer
   20 μL Ampligase at 5 U/μL
   12.5 μL dNTPs at 1 mM
   7.5 μL Nuclease-free water
When the thermocycler reaches the 60° hold (approximately 26 minutes), add 2 μL of enzyme mix to each sample and then advance the thermocycler to the next step (60° for 10 min)
Prepare the exonuclease mix:
   10 μL of Exo I at 200,000U/mL
   10 μL of Exo III at 200,000U/mL
When the thermocycler reaches the 37° hold, add 1 μL of exonuclease mix to each sample and then advance the thermocycler to the next step (37° for 30 min)
When the thermocycler reaches the 4° hold, add 25 μL of Phusion Master mix and 3.54 of each primer mix to every sample where the primers are at 7 μM. The primers are:
   5′CCATCTCATCCCTGCGTGTCTCCGACTCAGBBBBBB GGAACGATGAGCCTCCAAC-3′ where BBBBBB is a barcoding sequence to identify the individual sample. 5′-CCACTACGCCTCCGCTTTCCTCTCTATGGGCAGTCGGTGAT CAGATGTTATGCTCGCAGGTC-3′
Begin the amplification program on the thermocycler
   94° for 3 minutes
   20 cycles of:
      94° for 15 seconds
      60° for 15 seconds
      72° for 30 seconds
   72° for 4 minutes
   After amplification, pool and purify the products. Gel matrix purification or Ampure enrichment should enrich a product sized between 180 and 250 bases, excluding both primer dimers (˜70-90 bases) and self-ligated probes (˜160 bases). Ampure purification is performed as follows:
      Combine the barcoding reactions from above in a clean 1.7 mL test tube. This mixture is referred to as the “pooled PCR product”.
      Add 80 μL of the pooled PCR product into a clean 1.7 mL test tube.
      Invert the bottle of Agencourt® AMPure® XP Reagent (Beckman Coulter, P/N A63880) several times to mix.
      Add 64 μL (0.8×) AMPure XP to the pooled PCR product.
      Pipette up and down 10 times to mix.
      Allow the reaction to sit at room temperature for 5 minutes.
      Place the tube on a magnet such as the DynaMag™ Magnet (Life Technologies) for 2 minutes.
      Remove and discard the supernatant.
      While the tube is still on the magnet, add 200 μL of 70% ethanol.
      Leave the solution on the magnet for 30 seconds.
      Remove the supernatant.
      Repeat steps 9 through 11 once.
      Allow the pellet to dry for no more than 5 minutes.
      Remove the tube from the magnet and add 40 μL Nuclease-free water.
      Place the tube on the magnet for 1 minute.
      The purified DNA is located in the supernatant. Remove 30 μL and place it in a clean 1.7 mL tube. Although the AMPure resin will not interfere with downstream processes, it can interfere with quantification. Leaving 10 μL in the tube ensures that a minimal amount of resin carries over.
      Proceed to the Ion Torrent template preparation workflow. Typically 12-24 samples are sequenced simultaneously on an Ion Torrent PGM using a 316 chip.

This protocol produces a sequencing-ready library for the Ion Torrent PGM platform. The protocol can be easily adapted to other sequencing platforms by replacing the 5′ ends of the IonAmpF and barcoding primers with the adapter sequences for the platform. For example, to prepare the material for sequencing on the Illumina MiSeq, GAII, or HiSeq platforms, the following primers would be used:

5′-CAAGCAGAAGACGGCATACGAGATCGGTCTCGGCATTCCTGCTGAAC
CGCTCTTCCGATCTCAGATGTTATGCTCGCAGGTxC-3′
5′-AATGATACGGCGACCACCGAGATCTACACTCTTTCCCTACACGACGC
TCTTCCGATCTBBBBBBGGAACGATGAGCCTCCAAxC-3′

The use of this protocol with the Ion Torrent PGM machine allows for a clinical or other sample to be processed completely into an analyzed result in 14.5 hours as follows:
30 minutes DNA extraction from sample
2.5 hours for Protocol 1 and to quantify the resulting material on a Qubit
30 minutes to setup the OneTouch emulsion PCR machine
4 hours processing on the OneTouch
30 minutes to setup the OneTouch ES machine
45 minutes on the OneTouch ES
60 minutes of PGM initialization and chip loading
3.5 hours sequencing on the PGM
30 minutes basecalling
30 minutes data analysis

### EXAMPLES

### Example 1

### HPV Screening

Detection and accurate strain typing of HPV are important for assessing the risk of cervical cancer as well as for choosing therapies for various head and neck cancers. Thus, we used the methods of this invention to design a set of probes to detect and distinguish the following HPV types: 6, 11, 16, 18, 26, 30, 31, 33, 35, 39, 40, 42, 43, 44, 45, 51, 52, 53, 56, 58, 59, 62, 66, 67, 68, 70, 71, 73, 82, and 84. We sought a probeset that would reveal at least 20 variant nucleotides across at least four probes for every pair of HPV types. As HPV is a DNA virus, its mutation rate is relatively low. For example, a multiple sequence alignment of fifteen type 16 genomes indicates a nucleotide divergence of 2%. A multiple sequence alignment of sixteen type 18 genomes indicates a maximum nucleotide divergence of 167 out of −7850 nucleotides for a rate of 2%. Given the 2% genomic divergence and a 1% sequencing error rate, 20 informative nucleotides provides a specificity greater than 99.99%. Using the more conservative calculation that treats probes as the unit of observation, the four probes produce a specificity of 99.5%.

The resulting probeset contains 83 molecular inversion probes. The probe arms (5′ arm and 3′ arm) are listed below in Table 1. The complete probes are formed by appending the 5′ arm to the backbone sequence GTTGGAGGCTCATCGTTCCTATATTCCACACCACTTATTGATGATTACAG ATGTTATGCTCGCAGGTC to the 3′ arm and adding a 5′ phosphate to the molecule.

TABLE 1

| 5′ arm | 3′ arm |
|---|---|
| ACCAATAGGGCTTATTAAACAACTG | ATTGAAATATAAATTGTAAATCATATTC |
| CAATTGTGTTTGTCTTTGTATCCATT | TGTCTACACATTATTCCACAATC |
| CATATACCATTGTTGTGGCCCTG | TTGAAATATAAATTGTAAATCATACTC |
| GCCTTATGTAACCAATATGGTTTATTA | ACTGCAAATCATATTCCTCAAC |
| GCTGTCACTAGGCCGCCAC | CGTACCCTAAACACCCTATATT |
| GTGTCTAGCAAACATTTGTTCCTT | AAGCCAATATGGTTTATTAAATAATT |
| ACCAATATAGTGCTGCAACACCA | CATATTCATATGCAATATCACTTTC |
| GCTGTCACTAGACCGCCACA | ATACCCTATATTGATATGCAGAC |
| CCTGTGCACGTTGCAACCAA | TGTAGTTCATATTCCTCCACAT |
| GTAATATCCAACACAGCAGGTGT | TTGCATAGGTATTTCCTCATAGC |
| GCTGTAATACTGTTTGTCTTTCTATC | TAATGTCTACACATTGTTCCAC |
| AACAGTTAATAATCTAGAACTGCCAG | ACGTTGTGTTTCAGGATTATAA |
| GACATTTGTAAATAATCAGGATATTTAC | GTCAGAGGTAACAATAGAGCC |
| AATAAATGTCTAACAAACATTTGCTCC | TTGCAACCAATATGGTTTATTAAA |
| TCTATCTAAACTTATAGGATTTCCATCT | TATACAGGATTACCATTATTATCTAAT |
| CGTTGTAACCAATATGGTTTATTAAATA | TAAACTGCAAATCATATTCTTCC |
| GTCCAGTTTGTATAATGCATTGTATT | AAACACAGATGTAGGACATAATAT |
| AGTAATAGGGATGTCCTACGGCA | GCCTACCTCCAAACCTACAC |
| CTTGGAGGTTCAATTAACATGCG | GTTATATCATTATCAAATGCCCAC |
| ACAAATACCATTGTTGTGTCCCTG | AAATATAAATTGCAAATCATATTCCT |
| CACCTACACAGGCCCAAACCA | GTGGTTTGCAACCAATTAAAC |
| GGTGTAATATCCAATACCGCAGG | ACACTTCCATAGGTATTTCCTC |
| TGCATTTGGAAATTCAAATACTGTTA | GCAACGCACTTAAACGTTC |
| CATACATGTTTCAGGAATTGATAGTAA | CATTATCATATGCCCATTGTATC |
| CAAATACCATTATTGTGTCCCTGAG | CTGAAATATAAATTGTAAATCAAATTC |
| TCTATCTAAACTTACAGGATTCCCAT | TTATTTAATTGATATACAGGATTACC |
| AATTTCCTTCCAGTTTGTATAATCCA | CCCACATGTACTTCCCATAC |
| GTTACAGGACTAAAGGGTGTTCC | TCCGTGGCAACAACTTTGG |
| AAACAATGCCTGTGCTGTCTCT | GTATTGCCATACCCGCTGT |
| CTGCCCGTTTATAATGTCTACACA | TGTTTGCAGGTCCATATAATAC |
| TATTAGTGTCTGCCAATTGTGCA | CAATTTGCTTCCAATCACCTC |
| CAGGAGTTGTTGTAGAAGAGGAAG | ATACCTCCATAGGTATTTCCTC |
| CATTATCATATGCCCATTGTATCATTT | TGTATCCATTGTCCCATTGTC |
| AAATAAATGTCTAACAAACATTTGCTC | AAATACCATTATTATGGCCTTGT |
| GAGTGGTATCTACCACAGTAACAA | AGCATGAATATATGTCATAACTTC |
| ACCAAAGCCAGTATCAACCATATC | GTCTAACAAACATTTGTTCCCT |
| GCATCATCTAATAATGCTACCTTGG | ACTGTCACTCTACTATGTAAATAC |
| GATATACCTGTTCTATACCAGTATAATG | TGAAATGCCATATCACTTTCAT |
| CTGGCAAATAATTGTTCCCGGC | CCAGTATGGCTTATTAAATAGTTG |
| ATTAAACTCATTCCAAAGCATGATTT | AATACTTGTAGGATTTCCATCTAA |
| AATCCATAGCTCCAAACCCTGTA | GTTTATTATAATAGTGCCTAGCAA |
| CCTGTGCACGTTGTAACCAGT | GAAATATAAATTGTAATTCATATTCTTC |
| ACATATACCATTATTGTGTCCTTGTG | ACACAATTGAAATATAAATTGCAC |
| CATCCTCATCCTCTGAGTTGTCC | CCTAGTGTACCCATAAGCAAC |
| TAATATTAGACAAACCTGTTCTATACCA | CTGTGCATATTTATATGCTATGTC |
| AAATAATTGATTATGCCAACAAATACC | GAATTCATAGAATGTATATATGTCAT |
| GCTGTCATTAGTACGCCACAAA | CCCTATACTGAAAGGCAGATAC |
| ATATCTTTCCAATTACCACCATCATT | CTAGTTGAATTTACATACGAAATAA |
| ATATGCCCACTGCACCATGTC | GCTTTATCCACTCAGCCATT |
| TATATAAACCAAGGCGTGCCACA | AGCAGGCCTATGTAATGCA |
| AGATATACGGTATTGTCACTAGGC | TGCACCCTAAATACTCTATATTG |
| CAACACCTACACAGGCCCAGA | GGTACACAGCCAATAATACAC |
| CCATCTAATAGGTTTCTCATATATGTAT | AGTTCATATACTGCATTCCCAT |
| CTCATGCACCTTATTGATAAATTATATA | GCCGTGGTCCATGCATAT |
| CCAGTAAGGTTTATTAAACAACTGAG | ACTGTAATTCATATTCCTCTACAT |
| AATACTAGTATCAGGTAAACCAAATTTA | CATAACTGTGTCTGCTTATAATC |
| ACAACCAATAATGCATAATTGTGTTT | ATCCAATGGCACATCAGATTT |
| ACCAATCTATTATGTAAATAAGGCCA | CCTGACACACATTTAAACGTT |
| AATAATGCGCGGGCTGCCT | AGTATTGCCATATCCGCTGT |
| ACATAATTGTGTTTGTTTATAGTCCAT | TATCTAATGGAACATCAGATTTATT |
| GACAGTCTTTCAAAGAAACATTTCC | AGTAATACACTTTCCAATCGTAT |
| CTCCATAAGCTTCTTTGAATTTATATAA | TCCTCTGTCACACGTTAAAC |
| GTACACCTTATTGTCACTAGGCC | TACCCTAAACACTCTATACTGAT |
| GTCTACTATGTAAATACGGCCACC | CCTGTAACACATTTAAACGTTG |
| GTCCACTGAAACATTGTCCCTAC | AGCACCATATCCTGTATCAATC |
| GAGTGGTATCAACCACGGTAACA | AGTATGTATATATGTCATTATCTCTG |
| ATATCTTAAGAATTGTACTATGGGTCT | CTGGTGGAGTTTACATATGAAA |
| ACATATGCCATTATTGTGGCCCT | ATATATGTCATAACATCAGCTGT |
| GAGGAAGATATACCTTGCTGTCAC | CTCTAAATACCCTGTACTGATAG |
| CCTGTTCTATACCAATATAGTGCAG | CATATTCAAATGCCATATCGCT |
| AATTTCACTATCATCTGTTATGTCATG | AACATCTATATTGTAGCCATTGT |
| CTTTCGTCCCAAAGGAAACTGAT | TAACACATACAACATATACACAAA |
| AAGCATGATTTACCTGTATTTGGC | CTTATGGGATTTCCATCTACCA |
| ATTAATATCTAATATAGCAGGTGTGGT | AACAATAAATGTATCCATAGGAATT |
| ACAGATATGTTGTCCCTAACATCC | TGTCCACCATATCGCCATC |
| AAAGCTTTCAAATGCAGGATTATCA | TCCTGATAATAATGTATTCTAGCT |
| TCACAGTCGTCTGTTATATCATTATC | AATCACATCTATGTTGTATCCAT |
| GTTCCAATTTGCATCATTTAATTCATA | GCACTTTCATAACGTATATATTTC |
| TCTCTTCTTTAGTAATATCTATGTTGGA | TAAACGCTTGGCTATTGCTT |
| TCCACATTTATATCTTAATAATGCTAAT | TATAATTGTTAGTCTTTGTATCCAT |
| TATTCTTAAATCTGCAAATACAAAGTCA | GACAAATAATACATCTAATTAATATTTC |
| CTGTCAAATGGAAATGTATTTGGAAA | TATTACTGTCCAGTTCATAATAGT |
| ATATTCATCCGTGCTTACAACCTT | CAGGTAAATGTATTCTAAATACCC |

Analysis of the resulting probes against a set of 211 HPV genome sequences representing 77 types indicates that the probe set reveals at least 20 SNPs or 5 type-specific probes between every pair of the genomes taken from any pair of the 32 target HPV types.

These probes were applied to a set of ThinPrep and FFPE samples.

TABLE 2

| | This | PCR + | | | |
|---|---|---|---|---|---|
| Sample | Invention | Sanger | LA | TWI | Risk |
| 12 | 44 | | — | — | Anogenital Wart |
| 12T | 52 | 52 | 52 | ~52 | High Risk |
| 20 | 68 | 68 | — | A7 | Potential High Risk |
| 20T | 68, 70 | 68 | ~70 | ~68/70 | Potential High Risk |
| 80 | — | | 52, 59, 62 | — | |
| 152 | 58 | 58, 66 | 58 | ~58 | High Risk |
| 486 | 66 | 66 | 66 | 51/56 | Potential High Risk |
| 492 | 35, 59 | 35, 59 | 35, 59 | ~35, ~59 | |
| 505 | 56 | 56 | 56 | ~56 | High Risk |
| 515 | — | | | | |
| 522 | — | | | | |
| 523 | 44 | | — | — | Anogenital Wart |
| 536 | 16 | 16 | 16 | ~16 | Highest Risk |
| 537 | | | | | |
| 550 | 52, 30 | 52 | 52 | ~52 | High Risk |

Table 2: DNA from Thinprep cervical brush samples were assayed using three techniques: Roche HPV Linear Array kit, Cervista/Third Wave Invader technology, and a molecular inversion probeset (Table 1 or a subset thereof) containing probes targeting 32 HPV variants. The Roche and Cervista assays were performed as to manufacturer's instructions, and the, molecular inversion probeset was used with Protocol 1 and sequenced on the Ion Torrent PGM platform, 12-16 samples per sequencing run on a 316 chip. The results for HPV subtype identification are recorded and compared between technologies. In the table, a “~” before a type name indicates a truncation of the TWI or LA grouping that includes the named strain.

The results demonstrate cases in which the Roche and or Cervista technology are unable to determine the HPV subtype present with a sample, but the probeset produced by this invention identifies a HPV subtype present, and also cases in which discordance between Roche and Cervista tests is resolved by our test, which confirms the subtype present within the sample. Also illustrated is an example in which our test detects multiple HPV strains present within a sample, a case in which neither competing technology can accurately determine that both subtypes are present within the sample. Further, the data indicate the utility of broad panels, in that the Cervista and Linear Array tests do not detect type 44.

The final column of the table demonstrates the ability to stratify specific HPV type by previously assessed risk criteria, e.g. established pathological standard practice. Infections are classified by the type of condition most associated with (e.g. genital warts), or the calculated risk of developing cervical cancer.

### Example 2

### Bacterial Detection

In diagnostic or epidemiological settings, it is advantageous to be able to detect many species of bacteria simultaneously. For example, the species in Table 2.5 account for more than 90% of the healthcare associated infections in the United States. Thus, a kit than can detect all of these species at once offers substantial advantages over using individual tests. Furthermore, a test that can provide results in hours rather than the 2-4 days required by traditional culture techniques offers the possibility of earlier treatment or earlier detection of pathogen transmission in a healthcare facility.

TABLE 2.5

Staphylococcus aureus
Staphylococcus epidermidis
Staphylococcus saprophyticus
Acinetobacter baumannii
Enterococcus faecalis
Enterobacter cloacae
Enterobacter aerogenes
Enterococcus faecium
Klebsiella pneumoniae
Escherichia coli
Clostridium difficile
Proteus mirabilis
Pseudomonas aeruginosa

To detect and differentiate these organisms, a set of molecular inversion probes were designed using the invention disclosed herein. The probeset sequences genomic regions such that every pair of species is distinguished by at least 21 nucleotides from at least three probes. Furthermore, each of the three probes reveals at least four informative nucleotides. Thus, under a model of independent nucleotide mutation and a summed error rate of 0.15, this probe set is expected to provide a specificity of 0.9999. Under a worst-case assumption that all nucleotides within a probe are linked, the probe set provides a specificity of 0.94. To further differentiate these organisms, additional probes were designed to differentiate the various strains of each organism. The resulting combined probe set provides at least 20 differences or at least five species-unique probes for every pair of species, as determined by comparing all finished genomes for the target species available from Genbank.

The probe arms are listed below in Table 3. The complete probes are formed by appending the 5′ arm to the backbone sequence GTTGGAGGCTCATCGTTCCTATATTCCACACCACTTATTATTACAGATGT TATGCTCGCAGGTC to the 3′ arm and adding a 5′ phosphate to the molecule.

TABLE 3

| Probe arm 1 | Probe arm 2 |
|---|---|
| GCTGTCACCGTCCAGACGCTGTTGGC | TCCGTGCCTTCAAGCGCG |
| GACTCCGCAGAATACGGCACCGTGCGCA | GCGTACAGGCCAGTCAGC |
| GCAGTCGGTAACCTCGCGC | GCGCTATCTCTGCTCTCACTGC |
| GCTGTCCTGGCTGCAAGCCTGG | CCGAACTGCTGATGGACGT |
| GACAGCAGACTCACCGGCTGGITCCGCT | GCAAGATGCTGCTGGCCACACTG |
| GACAGAACAAGTTCCGCTCCGG | CACGGATACGCCGCGCAT |
| GCAATACCAGGAAGGAAGTCTTACTGCT | ACTAGTCATTGGAGTACAGATGATT |
| GAGGACCGAAGGAGCTAACCG | CGCCGCATACACTATTCTC |
| GCTGTAATGCAAGTAGCGTATGCGCTC | GAACAGCAAGGCCGCCAATGCCTGACG |
| GAACGTCTGGCGCTGGTCGCCTGCC | GCACAGGTGCTGACGTGGT |
| CGCATATGCTGAATGATTATCTCGTTGC | ATCTTGCTCAATGAGGTTATTCA |
| GACGACAGATGCAGGTTGA | CGCATCGCCGATGCTCATC |
| CGCCTGCTCCAGTGCATCCAGCACGAAT | ATGCTCTCCGCCATCGCGTTGTCA |
| AGTGCGTTCACCGAATACGTGCGCA | CAGGTTATGCCGCTCAATTC |
| AATCCAGGTCCTGACCGTTCTGTCCGT | ACCTCCGTTGAGCTGATGGA |
| GAGGTGGCCAACACCATGTGTGACC | GACGCCGGTATATCGGTATCGAGCTGCT |
| CGCATATGCTGAATGATTATCTCGTTG | ACGGTGATCTTGCTCAATGAGGTTATTC |
| GAAGTGCCGGACTTCTGCAGA | GCACGGCCTGATGGAGGCCGC |
| GCTAATCGCATAACAGCTAC | CATCACGTAACTTATTGATGATATT |
| GCTGCGGTATTCCACGGTCGGCC | GCAGGAACGCTGCCTGTGGTC |
| GAATCAATTATCTTCTTCATTATTGAT | CTGCGGCTCAACTCAAGCA |
| GTCACACGTCACGCAGTCC | GCATTCATGGCGCTGATGGC |
| GTGTTACTCGGTAGAATGCTCGCAAGG | ACTAGATGACATATCATGTAAGTT |
| CGGAACTGCCTGCTCGTAT | AACGATATAGTCCGTTAT |
| GCTCTCCGACTCCTGGTACGTCAG | GCGCGCATTAATGAAGCAC |
| GATGTTGCGATTACTTCGCCAACTATTG | GCTGTAATTATGACGACGCCG |
| CTCATTCCAGAAGCAACTTCTTCTT | GGATAGCCATGGCTACAAGAATA |
| GCAATACCAGGAAGGAAGTCTTACTG | GTCATTGGAGAACAGATGATTGATGT |
| GTATCGCCACAATAACTGCCGGAA | AACGATATAGTCCGTTATG |
| GCTGTGGCACAGGCTGAACGCCG | GGTGATGTCATTCTGGTTAAGA |
| ACATAATCTGAATCTGAGACAACATC | ACGCACTCTGGCCACACTGG |
| GTGAAGCGCATCCGGTCACC | ATGGCATAGGCCAGGTCAATAT |
| GGTTCTGGACCAGTTGCGTGAGCGC | CGTAACATCGTTGCTGCTCCAT |
| CGCTGGATTTCACGCCATAGGC | TGTCGCTACCGTTGATGATT |
| CGTATAGGTGGCTAAGTGCAGC | GTAACTCATTCCTGAGGGTTTC |
| GTACATACTCGATCGAAGCACGA | CCGGAATAGCGGAAGCTTTC |
| AAGGTCGAAGCAGGTACATACTCG | AGACATGAGCTCAAGTCCAAT |
| GAAGCTTTCATAGCGTCGCCTAG | TTAGCTAGCTTGTAAGCAAATTG |
| GAAGCTTTCATGGCATCGCCTAG | AGCTAGCTTGTAAGCAAACTG |
| CGCTACCGGTAGTATTGCCCTT | AGAATATCCCGACGGCTTTC |
| ATCGCCACGTTATCGCTGTACT | TTTACCCAGCGTCAGATTCC |
| CAAGTACTGTTCCTGTACGTCAGC | TCGCCAGTAACTGGTCTATTC |
| CAACGTCTGCGCCATCGCC | CGCAATATCATTGGTGGTGC |
| GCCGCCCGAAGGACATCAAC | CAGACGGGACGTACACAAC |
| CGTGCTGGCTATTGCCTTAGG | GTAATACTCCTAGCACCAAATC |
| CATTAGGAGTTGTCGTATCCCTCA | AATACTCCGAGCACCAAATC |
| AAATTGCAGTTCGCGCTTAGC | GTTCCATAGCGTTAAGGTTTC |
| GCGCCAAACAGACCAATGCT | GATTTCACGCCATAGGCTC |
| GTATAGGTGGCTAAGTGCAGCA | TCGTAACTCATTCCTGAGGG |
| GTCATCGCCTCTTCGTAGCTC | GCCATATCGATAACGCTGG |
| AGTATCTTACCTGAAATTCCCTCAC | CCTCTCGTCATAAGTCGAATG |
| CATCACGAAGCCCGCCACA | GCCCTTGAGCGGAAGTATC |
| ACCAATACGCCAGTAGCGAGA | GCAACGTAGCTGCCAAATC |
| CAATCAGTGTGTTTGATTTGCACC | TACCCGGAATAGCCTGCTC |
| CGGATAACGCCACGGGATGA | ACCGGGTCAAAGAATTCCTC |
| GCGGCGTGGTGGTGTCTC | CGCTGCCGGTCTTATCAC |
| GCCACGTCACCAGCTGCG | CGGCTGGGTGAAGTAAGTC |
| GCTCGTAGCGTCGCGTCTC | TTGACCGACAGAGGCAAC |
| CAGCAGGTCCGCCAATTTCTC | AGTGGACGTCAGTGCGC |
| CGTAGTGTCGCGTCTCCCG | CAGGATGAGTTGTGTAATAACTT |
| CCATAGAGGACTTTAGCCACAGT | TACACCGCTACAGCGTAAT |
| CATATGCAGAGTGAGCGGTCC | TCAATTCTTTCAAAGACCAGC |
| CCATTAACTTCTTCAAACGATGTATG | ACCCGTGCTGTCGCTAT |
| GTGCTGTCGCTATGGAAATGTG | AACCAAACCACTAGGTTATCTT |
| GTCAGTGTTTACAAGAACCACCA | ATGCATACGTGGGAATAGATT |
| CGGAAGTATCCGCGCGCC | TTCGATCACGGCACGATC |
| CGAACCAGCTTGGTTCCCAAG | TCACTGCGTGTTCGCTC |
| GATGCTGTACTTTGTGATGCCTA | CGCTTGGCAAGTACTGTTC |
| GCAAGAAAGCCCTTGAATGAGC | GCGTTATCACTGTATTGCAC |
| AATCAACAAACTGCTGCCGCT | GCTGTACTTGTCATCCTTGT |
| CCAGTCTGCCGGCACCGC | TCGAGCGCGAGTCTAGC |
| CCGACTGCCCAGTCTGCCG | CGAGCGCGAGTCTAGCC |
| GTAAATAGATGATCTTAATTTGGTTCAC | TTGCTGGCCAATCGTCG |
| CACAGCCTGACTTTCGCCGC | CAAGCAGGAGATCAACCTGC |
| GGTGGTCGATACCGCCTGG | GTGAAATCCGCCCGACG |
| CATGTCGAGATAGGAAGTGTGC | TGATGCGCGTGAGTCAC |
| CAATCTGCCATCGCGCGATT | CGGCAATCTCGGTGATGC |
| CGAAGCAGGTACATACTCGGTC | ACGAGCTAAATCTTGATAAACTT |
| TAGAATAGCGGAAGCTTTCATGG | AGCTAGCTTGTAAGCAAACTG |
| CAAGTCCAATACGACGAGCTAAA | GAATAGCATGGATTGCACTTC |
| GGTACATACTCGGTCGAAGCAC | AATCTTGATAAACTGAAATAGCG |
| GGTACATACTCGGTCGATGCAC | TCTTGATAAACCGGAATAGCG |
| GTAATTGAACTAGCTAATGCCGTAC | TTATGACACCAGTTTCTAGGC |
| CAAGTACTGTTCCTGTACGTCAG | GCCCAGTTGTGATGCATTC |
| TCTCTTTCCCATTGTTTCATGGC | TGCGGAAATTCTAAGCTGAC |
| GTAGGTTATGCAGTTATTAGGTTCAG | GACTCAGCCGAGTCAAGC |
| GCAGTACCAACATAGCTAAATGC | AAATAACAAATCACAGGCCAC |
| GGTCCTGTGGTGGTTTCCACC | CGCGATAATGGCTTCATTGG |
| TAACCGCTGTGGTCCTGTGG | TGCGCAATAATAGCTTCATTG |
| GGAAGCGTTGCTTGCCATAGT | AACCGAAGCACCATGTAATT |
| GTTCGGTGCAAAGACGCCG | TCGCAGACTTCAATATCAATATT |
| CACCTGATGCAGAACCAGCAT | AGGCCACGTTATCACTGTG |
| CAGCTGCCGTTGCGAACG | CGCAGATAAATCACCACAATC |
| GCTCAGACGCTGGCTGGTC | CCGCAGATAAATCACCACG |
| GCCAGTAGCAGATTGGCGGC | GAACGGGCGCTCAGACG |
| CCACTGCAGCAGATGCCGT | GTATCCCGCAGATAAATCACC |
| TTAATTTGCTTAAGCGGCTGCG | CCAGCTGTTCGTCACCG |
| GGGAAAGCGTTCATCGGCG | TCGCTCATGGTAATGGCG |
| GCGAACGGGCGCTCAGAC | ATAAATCACCACAATGCGCT |
| TCTTATCGGCGATAAACCAGCC | CGTTGCCAGTGCTCGAT |
| CAGTCCCTCGATATTCAGATCAGA | TTAACAATTTCGCAACCGTC |
| CAGCTGCGGTAAAGCTCATCA | CATAGTTAAGCCAGTATACACTC |
| GTCGGAAAGTTGACCAGACATTA | ATACTAGGAGAAGTTAATAAATACG |
| CATTCTCTCGCTTTAATTTATTAACCT | ATCGACCTTCTGGACATTATC |
| GTAACAACTTTCATGCTCTCCTAAA | CGGTAACTGATGCCGTATTT |
| GTGAAGTGAATGGTCAGTATGTTG | AGTGCGCAGGAGATTAGC |
| CCTGTCCTACGAGTTGCATGAT | ATAATGGCCTGCTTCTCGC |
| CGTTTCCAGACTTTACGAAACAC | ACGTTGTGAGGGTAAACAAC |
| CGTTGCTTACGCAACCAAATATC | TGATCTTGCTCAATGAGGTTA |
| CATCATGTTCATATTTATCAGAGCTC | TAGATTTCATAAAGTCTAACACAC |
| GTTTCCACATGGTGAACGGTG | AAACCTGTCACTCTGAATGTT |
| CAAATACTAAATTATACAGTATCAGAGAG | ATGCAAAGCGTTATGAAATTTC |
| GTTCTTATTATTATAAGTATCTATTAACAGTT | CATTAGTGGCTGCTGCAAT |
| CATCGGGAAATGGAAGTCGTTAT | GTTCAATCGTCAAAGTTGTTC |
| CGTGGTTTGTGCTGAGCAAAG | CAAAGTTAAGTTGTCAGTTTGAG |
| GCCGCCCGAAGGACATCAA | AGACGGGACGTACACAAC |
| GCAACTCATCACCATCACGGA | TGATGCGTACGTTGCCAC |
| GCGACAGCCATGACAGACGC | GGACAATGAGACCATTGGAC |
| AAACGACTGCGTTGCGATATG | TTCCGAAGGACATCAACGC |
| ATGCGACCAAACGCCATCGC | ATCGTCATGGAAGTGCGTA |
| GTCATGAAAGTGCGTGGAGACT | ACCGGGATAGAAGAGCTCT |
| GAACAGGCTTATGTCAACTGGG | CATAACATCAAACATCGACCC |
| ACGAACCGAACAGGCTTATGTC | TAACGCGCTTGCTGCTT |
| GCTGTAATTATGACGACGCCG | CTCGGTGAGATTCAGAATGC |
| CATCATAGACGCGGTCAAATAGA | ACTCATCACCATCACGGAC |
| GTGTATGTCAGCGATTTGTCCAT | TGTCATATTGTCTTGCCGATT |
| GTCCACCTCGCCAACAATCAA | ATATCAACACGGGAAAGACCT |
| GCGTGATTATCACGTTCGGCA | CTTGCAGATTTAACCGACAC |
| GGCTCGACTTCCTGATGAATACG | TGAAACCGGGCAGAGTATT |
| CAACGATGTATGTCAACGATTTGT | ATTGCGTAGTCCAATTCGTC |
| CAGGCTGTTTCGGGCTGTGA | GGGTTATTAATAAAGATGATAGGC |
| GGCTCGGCTTCCTGATGAATAC | AGGCATGGTATTGACTTCATT |
| TAATTCAAGTGCAACTCTCGCAA | TTTATTCTCTAATGCGCTATATATT |
| GGATAGTTACGACTTTCTGCTTCA | TGTATTGCTATTATCGTCAACG |
| CAGTATTTCACCTTGTCCGTAACC | GTTTACGACTTGTTGCATGC |
| AATGTTTATATCTTTAACGCCTAAACT | ATGCTTTGGTCTTTCTGCAT |
| CTGGCCCTTGAGGTCGCGG | CGGTCTTCACCTCGACAC |
| GACGTAGATCGGGTCGAGCT | ACGGAAACCTCGGAGAATT |
| GGCGTACTGCTGCTTGCTCA | TGACGTCGACGTAGATCG |
| CCTGTTCCTGGGTCGAAGCC | CTTCGGTCACCGCGGA |
| GTCAGGCTAAATATAGCTATCTTATCG | TCAGTTACTGCTATAGAAATTGAT |
| CATCCTAAGCCAAGTGTAGACTC | AAGATATATGGTAATATTCCTTATAAC |
| GTTTATAAGTGGGTAAACCGTGAAT | GAAACGAGCTTTAGGTTTGC |
| GCAGCACTTGACCGCCATGAGTGACCA | CATCGCACCAACAACAATAATCG |
| GTGATCACTGATGCACCAGATGAAGT | ATCTTGATATTCAAGTCTATGACG |
| GATATTATTGATCATGGTGCCAAGCCAA | CAATATGAAGCTGACGACGCG |
| GCTGAGCGTGAAGGTTCATGGATTATTA | GGTAAGGCTTACGGTCTCAT |
| GCATCTTGTGCAGCCTGAATAGCAGCGT | ACCACGTTGAATATCACCTTCGGCAT |
| AAGTCCATAATTGCTTGAGTGTAGTCAT | ATCTTCGCACTGAATAATAAGAACAT |
| GCTTGCTGGTTCTGCACGTAGCTTACTG | AAGATGAACAGGCTACTGCAA |
| GCAGCGCTGTGCAAGTTCAATGTATTCT | CTCGTGCGAGTATTCCTTAAGTGT |
| GTATAACACTCGGCCAGCGCCAAGGTTC | GTTCACACATCGCCACAATATGAT |
| ACCATGCAGATACAATGAACCA | GGATGATAAGACACATCCAATTC |
| CATCAACAGCTTCTTGAAGCATTC | GTCCAACAACTATAACAGAACGTC |
| AACATATCACCTGATATTCTAGTATC | ATTCCATTATATTCAACAGGATTGTGA |
| GCTGTTGCTTGCGGATACTG | CGTATATGTAGCTCAAGTTGC |
| AAGAGCTAATGCAGCTATTGCACTTAT | CATACACTTCAGCTATAAGACCAT |
| AACAAGAGCAGAAGTTACAGACGT | GTATAATGGTGGCTAGAGGTGA |
| ACTCGTGAAGACCATGCAGATACAA | AATACTTACAATGCCTGAGGA |
| ACCATGCAGATACAATGAACC | CCTGAGGATGATAAGACACATC |
| GCATCTGCTGCTTCTATTGCTCCTACT | ACATGAACTGATATTAGTTCTCCAA |
| GCACAAGCTGGAGATAACATCGG | GTAGAGGACGTATTCACAATCACT |
| CTCTATCAGCTTCTACTGCTTCTTC | CCATCTCATCCACAGTTAATATATC |
| AGATGAGATTCATACTATCGTTGGAGCT | AGCAGAGAGAATAGTAAGAGGAGA |
| CATCAACAGCTTCTTGAAGCATT | GTCCAACAACTATAACAGAACG |
| GTCAGCAATACGCCACCAAGCTCCTAT | GTGGTGGATATCCTGTTACC |
| GCGCAATAGAGTTGTATAAGAGTGCTG | AGCATTAATTATAGATTATAATGTATAA |
| GGCATAATAGGATGGATAGATGA | ACTAATCCAACTTCTACTGCTAT |
| GTACATTCACATATAGACCATCTTAA | ACATAGGTGCAGGTAGAATAGTATA |
| CCATACCAGTATCTTGGCATATTG | ATAATGAATAACAGCAGGTGTATTA |
| AGATGAAGCACAAGCTGGAGATAA | AGGACGTATTCACAATCACTG |
| ATAATCATTCACCTCCATCATTCATAA | ACTGAATATGGTTCGTCTCA |
| GTACATTCACATATAGACCATCTTA | ACATAGGTGCAGGTAGAATAGT |
| ACTCCACCAGGATGTTGTCC | GTAGGACCGTCGTGTCCAAG |
| GCAATATCAATGGTATCGAAGGCACTAT | GTATTGAAGGTACTATTAGCGATATGC |
| GTGCCGGTCTCGGTTACTCAATG | GGATTATTATAATGCAGCTAGAAG |
| GTACATTCACATATAGACCATCTT | ACATAGGTGCAGGTAGAATAGTA |
| AGTTCCTTCATATGACTCAGTTGATTGA | GTTATATCTTCAATTATACATTCCTGC |
| CAGCAGTTGTTGCTAGAGGTATG | GCATCACCAGGTGCAGCAAGT |
| AGTGGTGAAGGTGTTCAACAAGC | ACTGAAGCTGGATATGTTGGAGA |
| GCAATTCTCTGTTGTTGTCCTCCACTCA | AGTAAGAGCCTCTTCTTGGTCATGA |
| CTATTCCTGATAATAAGTGTGTCCTCAT | CGGCATCATCTAACAATTCTTCT |
| GTAATTCCAATTACTTCTAGCTCTGGTG | TACCATCTTCTCCATGTGTAT |
| CCATGCAGATACAATGAACCAG | GATGATAAGACACATCCAATTCC |
| CCTTCTGCCATTGTAGAACAAGCTCCAT | CCTGTAACTGTCCACTGAGC |
| CAATCATGATAGAATTAGATGGAAC | AGCAATAGTTCCATCAGGAGCATC |
| AGTGGTGAAGGTGTTCAACAAG | ACTGAAGCTGGATATGTTGGAG |
| CGCCTCTTCAGAAGCGGATATCA | GCCAGACTTCCGCCACAACCT |
| GGCATAATAGGATGGATAGATGAGC | GCAGCAGTTGTACCTACAACTAA |
| AGTTCCTTCATATGACTCAGTTGATTG | GTTATATCTTCAATTATACATTCCTGCG |
| GCATGGTAGTTCGCCAGCCGCTGGAAC | ACAGCAACCGCAAGTTCTTGACAT |
| AATATCATGGTCGTGTCCAGGCACTGGC | GTTCTGGTAGCTGCTTCTACTGTA |
| AACTTACAACTACGCGCACTTGAATCG | GAGTGTTGTATGATAGTCTCGGT |
| GCAAGTTGAGGAGATGCTGGCATGATTC | ACATGGCTCTGGAAGATGTGCTGATC |
| GCGATAATTGTAATGATTCGTGGTGTTA | CCGTTGTCAATCCAGTTAGTAGACT |
| ACTGTGGCAGTCTATGTTCCAATTGTA | CTTATCGACATAATCCTGATAATC |
| GCGTCGCTTCTTGCGCTCGCC | AATGTATTCATACCGTCAAGT |
| GCCTTCACAACTACGTTGGAAGGTCTTC | CTAACAGTCCTGCCGACTAC |
| GCCTTCACAACTACGTTGGAAGGTCTT | CTAACAGTCCTGCCGACTACT |
| GCCGCTGAGCGGCGGCAAGCCGATGGC | GAATGGCAGGCCAAGCTGAAGGCG |
| GCCAAGCGGCATTCTGGCGCCAGTGGA | CCAGACCGGAGTGGACAACGTCGAGGCG |
| GCCGTATATCATCGGCAATAACCGCACG | GCATGATGGTCAACAAGGTGC |
| ACGAGCCGAGATAGGTCTGCAGCGTAC | GTACTGATATTCACCATACTGCCG |
| GCAATATCTTCACCGGCAGCCACCGCG | GGTATATGGCACGCCAATCGC |
| AATAACCTTAACGTCGCCAACACG | CTCGGTGAACACCTCCTGGCACG |
| GCGGAACTGCTTGGCGTAGTAAGC | CATGTAGTGCCGTAGACCTTCACCA |
| GCGAGACCGGCGGCACCATCGTCTCCAG | TTCTGCCTGATGGACGTCTCCGGCTCG |
| GCGGTTCACCTGTTCGCCTTCGAACACG | GCGCAGCATCTGACGCAGGATGGTCTCG |
| ACTCCATCGCCATCAAGGACATGGCCGG | ATCGACGTGTTCCGCATCTTCGACGCG |
| GCCTGATGCACTACAGCGCCTGG | TACCACATGGTCGATCTCGACGACTGC |
| GCGCATCCAGGACGGCGAGTACG | CTTCGAGTGCCTGCACGAGCTGAA |
| GCTGGAGAACGTCAAGGTGGTGATCATC | ACCGATAACGACGACCGCATCAA |
| ACGATTGGAGAAGGCAGTGTGATTGG | GGACAGATTACAATTGGCG |
| GCCGCAATACCGATATTCCA | CCATTGTCCACCAGCTGAACCG |
| GTGAAGGTCGTGCTCCTATCGGT | AGATCTGGTGAAGTTCGTATGAT |
| GCTGGTACTTGTACTTATATCGA | ATCAGAAGATGATATCGTTACGTCAT |
| GCGCATATTGCATTAATGGCTATAGAT | GCCAGCAGGTTATACACTCG |
| GCAATTCTTACCACAGCACGAAGAACAG | ATCTAGATGAAGATAATGAAGTCG |
| GCATCTTCATACAATACTTCTAGCTTAC | CACAATACCAGTTGTATTACG |
| GCTTCAGCGCCATTACCGCCACCAGCT | ACTCTTGATATATTCTTGTAAGCG |
| GTTCACACAACGCGCCGACTAGAATCC | CACGATATCCAAGATAATGATTGGCTA |
| GCGCACCTACAATCGCCATTACTACAC | ACTCATTATCGACTGTTACATCGACTGA |
| AGCGCACATGTGACAGCGTGTAGGTTA | GTGCCTTAGATTGTTCAGAACAAT |
| CGAATGGATATGTACCATGGTCGATATC | CTCTCTAATATGATGTCCAT |
| ACTACAACAGCAACCGCATTACAATGGC | GGTGCTAAGAGGTCATCGGA |
| AGCTTCAGATAAGTACCTATCTGA | GGAAGAATAGTTATTCTTGATAATGTAT |
| CGTATTGCTCGAATACATGATA | ACAATGTATCAAGGCCAGCT |
| GCGACCAGTTGTTATCGACCGTGT | CAGAACGATACGGTGCTGTATA |
| CAATTACATTGTCTGTTGCGTAGATACC | GTTGTGGCTAATGTGCCAGTT |
| GCACCACTCTATAGCAGTAGCGTATTG | ACAGCCAATGTCACCTAAGTCAACA |
| ACAGTCCGAATAAGATACGACTATTCGA | CGTTGTAACGTATATGAATAGTTGA |
| AGATGCAATAACAGGTCGAATATTAATT | GCCATAGTGAGAGTAGTGAA |
| CAATAACAGGTCGAATATTAATTAATTG | GCCATAGTGAGAGTAGTGAAC |
| AGATGCAATAACAGGTCGAATATTAA | ACACATACGGCCATAGTGAGAG |
| GAACATAACGCGACGTTCCAGCTG | GCTTCAGAGGTGTTGTAGTCG |
| GCGCTGGCGCAGTATCGTGAACTGG | ACCAACGTAATCTCTATTACCG |
| GCTGTAATGCAAGTAGCGTATGCGCTCA | AAGGCCGCCAATGCCTGACG |
| GCCTGTAGCAACAGTACCACGACCAGT | CACCACGTAATAATGCACCAA |
| ACTACGCTGAAGCTGGTGACAACATTG | GTTGAGGACGTATTCTCAATC |
| GCTGGTACTTACGTTCAGAT | ACGGTGAACGCCGTTACATCC |
| GCAATTCTTACCACAGCACGAA | ATCTAGATGAAGATAATGAAGTCG |
| GCGGCGGCAGGCGGTAACGCCAG | ACGCGGTTATCTACCACGGCG |
| GCACCTACTTGTCCAGCACCAGCCAT | AATACCACCACCAATACAAGCA |
| GCGCGGTAACATGCCATATTCTGC | CCTGAATGACATCACAGTCG |
| AATCAGGTCAAGGAACTGCAAGC | GTCTCAATCATATGCACCGGAATAC |
| GAACATATGTGTATGACGATGCGCGG | GTACATGTCGCTTATCTGCCAGAAGGT |
| CGTGTGCGTAGTGACGAGTTGGAGA | AGAATACGATGATGTAAGGTACACCTA |
| CAGGAGTTACTTCTGTTCCAT | TTGAACAATTAGATCACCTCG |
| CGTAATCTCCATTACCGATGGTCAGATC | ACGTATTCTACCTCCACTCTCGTCT |
| CATTCGACGTTCTGGTATTACTT | CACGCTCCGCATCAGCAGCACCACGTT |
| CTGAACCACGGATTACTGGAGTGTC | GCCTGTTACTACTGTACCACGAC |
| GAATCGAACGGTCTCATTAACAGAT | GCTTTCCAGGGATATAAGACGC |
| CCCGCAGAGTCACACTCGGA | ACTCTTGGTACTACTCACTAGC |
| GAGTCTCTTTCAACCTGGATTAGATAT | AAGATTAATAGCGTACTTTACTCC |
| ATCCCGCAGATACTAGGTTCTTAAT | GAACTATTCATATTACACCCTAAGG |
| CAGTGGGCTATCCTAAGCCAAAG | CATAAGCGAACTAACTATCACTTA |
| ACAAAGCGTTCTAAACGATTAGAACT | CGAGAAAGGAAACAGGATAGTAC |
| CCAATGGAGAAGTCTAAATGTCCAA | TTATCAGAGATACATGACTCTTAGG |
| CGAATCACTGGACTACATTTATATTTCT | AGCGAACCTTTATATTTGACCAT |
| CTCAAGTCTTGCCCTGATAGAATTAT | TCACGACTTATCTACTTTAGAAATC |
| AGTGTTAGGTCTTTATTAATTAGCCCA | TTTGATTTGCCTATTGAGAAATTAA |
| GGTGATCGTTATTATGATAGTACGGC | CTCGGTTAAGGGAATTACGAC |
| ACTCGGATGGTAGGTTTATTAAAGC | GTGATCGTTATTATGATAGTACGG |
| GGAGCGGTAACAAGTTTCCACC | GGAATATTGTTGGATTTAAAGACAA |
| ACAATCGTTGTCGCACTGCATAG | GAACTTGGTCTACCGTACCAC |
| GGATAATACAATCCTAATACGTACGGA | GCTGCTGTAACTAGGGTAGC |
| CTATATTCAACGGGTCACGGGTAG | TCATTGATTCGATCTCGTAACTC |
| AATGTTATTGTGGTTGCGTGTTCG | TACTTTGGAAGTGCCCTGAC |
| CATGTCTTCTAGTACAGGTTTGCCG | TGTAAGAGGCCGCTAACTTC |
| CTCTGGCTCGTGGGCTCGG | TTCTTGAGATAGTCCGGTATAATC |
| ATTCGATCACGATGGGCTGGG | AATTTCCTGTGTCATACACGC |
| CAATTGATTTAGCCACTACACCTTAC | CACTATTCTGGCGACCACC |
| GATAAAGAAGCGTCTTGACCCAGT | ATCTGGTGCTCCTTGACGC |
| GCAAATTTAGAGAGTGCATGCATG | GGAAGAGGACGGCATACAAC |
| CATTTCATCTAGACCGCTCGTGT | GCTTGAAGTGTATGTTGGGAC |
| GTCGCCCTCGTGCTAACGT | GGTTCTTTGATGTACCGGTT |
| GCTGATGACGGTGAAGTTTATCA | CATTATCGCACATATTGACCAC |
| GAAATTAGCTAAAGGGATATCGCG | AACTTTCCGCCAATCCTGC |
| CACCTACGTTCTCACCTGCAC | ATTCGATAGTACCAGTTACGTC |
| GTTGCTTATAGCGTCGCTGCT | CTGGTTATCGAGAAGATAAAGG |
| GTAAGCGTAGCGATACGTTGAG | GAGTGAACGCACCACTGG |
| TCAGGTAGAGAATACTCAGGCGC | CGGAGAAGGCTAGGTTGTC |
| GCAACCCACTCCCATGGTGT | CGTTCTTCATCAGACAATCTG |
| GCCCTTTCAGGACTTTGATACTGG | TGTACGGAGACGGAGTTATCG |
| ACACTGACCGATTCATCCTCGTG | CTTGAAAGTGCGTTAACAACC |
| CGGAAGCCCACCAAGTGAGTAC | CGAAACCAGTTTGTCCTTAGTC |
| ACCAGCTTGTCTTTAGTCTGAGAG | CTTTACGACGGGTCATTTCAC |
| CATTGGTTTGTTCTGTTTGAGAGGC | GATTCATCTTCGTGAATTGTGAC |
| GGACTTTGATACTGGAGGAGTCATA | TGTACGGAAACGGAGTTATCG |
| ATGCTGGAGGAGTCGTACGTTT | GTCGCGCACACTAATAGATTC |
| AACTAAACCTACACGGAATTGGTTC | GCAGATACACGACGTTTATGT |
| GCCGCTTCACCTACGTTAGGAA | CGTAAAGATGAGTCTTTAACGTC |
| GACGTTTGTGCGTAATCTCAGAC | GAGGAAACCGTATTCGTTCGT |
| ACAACACTTTACCACTTGAGTGGG | GTAACTGCCCATGTCAAGATAC |
| CCACGTTTAGTTGAACCACCGC | TCAATACGCCAGTTGTTAGTTC |
| AATCGATAATAAGTACGGTGCATCC | GAAGAATACATTCGCGTACATC |
| AAGCAAGATCGAGTCTTCATAGTTG | GATATACACGATACCTGATTCGT |
| CCGATATTCATACGAGAAGGTACAC | CAGTAACTCTATTGTCAAACGGT |
| GTAGTGAGTCGGGTGTACGTCTC | TCTTCGATAGCAGACAGATAGT |
| ACCTACACGGAATTGGTTCTCAGT | GATACACGACGTTTGTGTGTA |
| CAACATCATTAGCTTGGTCGTGGG | TTGCGTGTTACCAACTCGTC |
| CGGCACGTCCGAATCGTATCA | TCGTGTCCCGTATATGTTGG |
| AATAGAGGCCCACAAGTCTTGTTC | CGCTCTCCACTATGGGTAGT |
| GCTACATTAATCACTATGGACAGACA | GATGGTCGATCTATCGTCTCT |
| GAAGTGTTATTCAAACTTTGGTCCC | CTTGAACCCTTGGTTCAAGGT |

TABLE 4

| | | | | | | | | | | | 5 × | | | |
|---|---|---|---|---|---|---|---|---|---|---|---|---|---|---|
| | | 10\hat{ }5 | | | | 10\hat{ }4 | | | | | 10\hat{ }3 | | | |
| | | copies | | | | copies | | | | | copies | | | |
| | Sample | rep | rep | rep | rep | rep | rep | rep | rep | rep | rep | rep | rep | rep |
| Organism | Type | 1 | 2 | 3 | 4 | 1 | 2 | 3 | 4 | 5 | 1 | 2 | 3 | 4 |
| S. aureus | Pure | Pass | Pass | Pass | | Pass | Pass | Pass | | | Pass | Pass | Pass | |
| | Culture | | | | | | | | | | | | | |
| S. epidermidis | Pure | Pass | Pass | Pass | | Pass | Pass | Pass | | | Pass | Pass | USER | |
| | Culture | | | | | | | | | | | | | |
| S. saprophyticus | Pure | Pass | Pass | Pass | | Pass | Pass | Pass | | | Pass | Pass | Pass | |
| | Culture | | | | | | | | | | | | | |
| P. aeruginosa | Pure | Pass | Pass | Pass | | Pass | Pass | USER | USER | Pass | Pass | Pass | USER | USER |
| | Culture | | | | | | | | | | | | | |
| E. coli | Pure | Pass | Pass | Pass | | Pass | Pass | Pass | | | Pass | USER | Pass | |
| | Culture | | | | | | | | | | | | | |
| E. faecalis | Pure | Pass | Pass | Pass | | Pass | Pass | USER | Pass | | Pass | USER | Pass | Pass |
| | Culture | | | | | | | | | | | | | |
| K. pneumoniae | Pure | Pass | Pass | Pass | | Pass | Pass | Pass | | | Pass | Pass | Pass | |
| | Culture | | | | | | | | | | | | | |
| E. cloacae | Pure | Pass | Pass | Pass | | Pass | Pass | Pass | | | Pass | Pass | USER | |
| | Culture | | | | | | | | | | | | | |
| A. baumannii | Pure | Pass | Pass | Pass | | Pass | Pass | Pass | | | Pass | Pass | Pass | |
| | Culture | | | | | | | | | | | | | |
| E. aerogenes | Pure | Pass | Pass | Pass | | Pass | Pass | Pass | | | Pass | Pass | Pass | |
| | Culture | | | | | | | | | | | | | |
| E. faecium | Pure | Pass | Pass | Pass | | Pass | Pass | Pass | | | Pass | Pass | FAIL | |
| | Culture | | | | | | | | | | | | | |
| C. difficile | Pure | Pass | Pass | Pass | | Pass | Pass | Pass | | | Pass | Pass | Pass | |
| | Culture | | | | | | | | | | | | | |
| P. mirabilis | Pure | Pass | Pass | Pass | | Pass | Pass | Pass | | | Pass | Pass | Pass | |
| | Culture | | | | | | | | | | | | | |

USER = user error
FAIL = no detection

Table 4: A molecular inversion probeset (Table 3) designed to detect 12 common bacterial pathogens was used to assay pure genomic DNA isolated from each of the 12 pathogens using Protocol 1, and the resulting sequencing libraries sequenced on the Ion Torrent PGM. Each genomic DNA sample was assayed in triplicate at 3 different copy number amounts in the molecular inversion probe assay. The results were analyzed using software that implemented the methods described in this disclosure-namely, assigning sequencing reads to the best match genome from Genbank. Pass criteria indicated detection of >1000 reads of the target pathogen, with less than 100 reads of an unexpected pathogen from the pure gDNA samples. User errors were identified in cases of manual error or sample mix-ups, or failure was indicated if the sample did not meet the pass criteria. The table indicates that of 139 samples tested, there were 9 cases of user error, and only one case of assay failure. There were no cases in which the sample pathogens were misidentified as another species. This indicates a >99% sensitivity and specificity for this assay.

This probe also detects many drug resistance genes, including most beta-lactamase enzymes, mecA, erm, vanA, and mex. Thus, it may be used to stratify patients for various purposes:
isolation or quarantine groups. Patients carrying identical drug resistance genes may be placed nearby in a health care facility to minimize the spread of the particular drug resistance gene to previously susceptible organisms.
Isolation or quarantine procedures. The presence of certain organisms or their drug resistance genotype frequently indicates that contact-isolation procedures should be taken to prevent the transmission of the organism to other patients in a health care facility.
Treatment stratification. Patients whose sample produces similar species or strains or similar drug resistance genotypes may be treated similarly. A physician might use information about which therapy was most effective on previous patients with an identical or similar pathogen.
Treatment selection. The presence of certain antibiotic resistance genes recommends against the use of certain antibiotic drugs. Similarly, certain species or strains are known to carry drug resistance genes such that identification of the species or strain recommends against the use of certain drugs even if the drug resistance gene is not explicity detected.

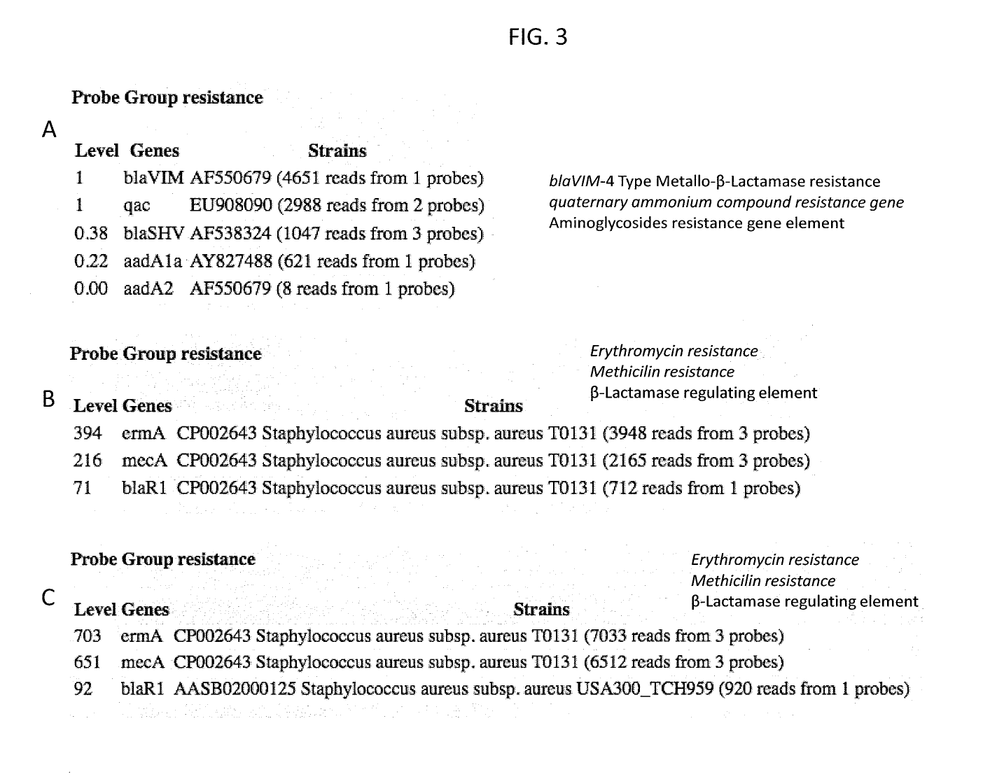

FIG. 3 shows three examples of drug resistance detection from clinical isolates.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

Unless defined otherwise, all technical and scientific terms used herein have the same meaning as commonly understood by one of ordinary skill in the art to which the invention belongs. Any methods and materials similar or equivalent to those described herein can be used in the practice or testing of the invention.

For all patents, applications, or other reference cited herein, such as non-patent literature and reference sequence information, it should be understood that it is incorporation herein by reference in its entirety for all purposes as well as for the proposition that is recited. Where any conflict exits between a document incorporated herein by reference and the present application, this application will control. All information associated with reference gene sequences disclosed in this application, such as Gene IDs or accession numbers, including, for example, genomic loci, genomic sequences, functional annotations, allelic variants, and reference mRNA (including, e.g., exon boundaries) and protein sequences (such as conserved domain structures) are hereby incorporated herein by reference in their entirety.

The publications discussed herein are provided solely for their disclosure prior to the filing date of the present application. Nothing herein is to be construed as an admission that the invention is not entitled to antedate such publication by virtue of prior invention. Further, the dates of publication provided may be different from the actual publication dates, which may need to be independently confirmed.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g. elements, including numerical ranges and exemplary embodiments) of particular embodiments and aspects of the invention including the working examples. For example, particular experimental parameters exemplified in the working examples can be adapted for use in the claimed invention piecemeal without departing from the invention. For example, for materials that are disclosed, while specific reference of each various individual and collective combinations and permutation of these compounds may not be explicitly disclosed, each is specifically contemplated and described herein. Thus, if a class of elements A, B, and C are disclosed as well as a class of elements D, E, and F and an example of a combination of elements, A-D is disclosed, then even if each is not individually recited, each is individually and collectively contemplated. Thus, is this example, each of the combinations A-E, A-F, B-D, B-E, B-F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus to the extent an element is described in one or more references known to the person having ordinary skill in the art, they may be excluded from the claimed invention by, inter alia, a negative proviso or disclaimer of the feature or combination of features.

The described computer-readable implementations may be implemented in software, hardware, or a combination of hardware and software. Examples of hardware include computing or processing systems, such as personal computers, servers, laptops, mainframes, and micro-processors. In addition, one of ordinary skill in the art will appreciate that the records and fields shown in the figures may have additional or fewer fields, and may arrange fields differently than the figures illustrate. Any of the computer-readable implementations provided by the invention may, optionally, further comprise a step of providing a visual output to a user, such as a visual representation of, for example, sequencing results, e.g., to a physician, optionally including suitable diagnostic summary and/or treatment options or recommendations.

While this invention has been particularly shown and described with references to example embodiments thereof, it will be understood by those skilled in the art that various changes in form and details may be made therein without departing from the scope of the invention encompassed by the appended claims.

1. A method of assembling a panel of capture primers for high specificity multiplex organism detection by nucleic acid sequencing comprising the steps of:
providing an estimate of the error probability of nucleic acid sequencing;
providing a desired level of minimal high specificity;
determining the number of polymorphic loci required to achieve the desired level of minimal high specificity by calculating a cumulative distribution function using the estimate of the error probability; and
providing a plurality of capture primers that each capture a region of interest comprising the number of polymorphic loci required to achieve the desired level of minimal high specificity.

2. The method of claim 1, wherein the nucleic acid sequencing is selective sequencing, wherein the sequenced loci represent less than 5, 4, 3, 2, 1, 0.5, 0.4, 0.3, 0.2, 0.1, 0.05, 0.001%, or less of the genome of two or more genomes of organisms to be detected in a sample.

3. The method of claim 1, wherein the plurality of capture primers are provided from a collection of potential capture primers.

4. A non-transitory computer-readable storage medium that provides instructions that, if executed by a computer, will cause the computer to perform operations comprising the steps of claim 1.

5. A computer comprising the storage medium of claim 4 and a processor for executing the instructions.

6-8. (canceled)

9. A panel of capture primers for:
a) high specificity multiplex detection of HPV (human papiloma virus) by nucleic acid sequencing comprising one or more of the sequences in Table 1, or their reverse complement; or
b) high specificity multiplex detection of a plurality of bacteria species by nucleic acid sequencing comprising one or more of the sequences in Table 3.

10. The panel of claim 9, comprising at least 2, 3, 4, 5, 10, 20, 30, 40, 50, 60, 70, 80, 90, 100, 110, 120, 130, 140, 150, 160, or all 166 of the sequences in Table 1, or their reverse complement.

11. The panel of claim 9, wherein the capture primers are circularizing capture primers.

12. (canceled)

13. The panel of claim 9, wherein the capture primers are conventional primer pairs.

14. (canceled)

15. A method of high specificity multiplex detection of HPV (human papiloma virus) by nucleic acid sequencing comprising contacting a test sample suspected of containing HPV with the panel of claim 9, performing a capture reaction, sequencing the products of the capture reaction, and analyzing the sequencing results to determine the presence of HPV and, optionally, determining the strain of the HPV.

16. The method of claim 15, further comprising identifying a suitable treatment on the basis of HPV detected, and optionally providing the treatment to a subject from which the test sample was obtained.

17. (canceled)

18. The panel of claim 9, comprising at least 2, 3, 4, 5, 10, 20, 30, 40, 50, 60, 70, 80, 90, 100, 110, 120, 130, 140, 150, 200, 250, 300, 350, 400, 450, 500, 550, 600, or all 610 of the sequences in Table 3, or their reverse complement.

19. The panel of claim 9, wherein the capture primers are circularizing capture primers.

20. (canceled)

21. The panel of claim 9, wherein the capture primers are conventional primer pairs.

22. (canceled)

23. A method of high specificity multiplex detection of one more of the bacteria in Table 2.5 (e.g., at least 1, 2, 3, 4, 5, 6, 7, 8, 9, 10, 11, or all 12) by nucleic acid sequencing comprising contacting a test sample suspected of containing one or more of the bacteria with the panel of claim 9, performing a capture reaction, sequencing the products of the capture reaction, and analyzing the sequencing results to determine the presence of the bacteria and, optionally, determining the strain of the bacteria.

24. The method of claim 23, further comprising identifying a suitable treatment on the basis of the bacteria detected, and optionally providing the treatment to a subject from which the test sample was obtained.

25-33. (canceled)

34. A method of detecting an a non-nucleic acid biomolecule by nucleic acid sequencing comprising the steps of contacting a non-nucleic acid biomolecule comprising an associated predetermined nucleic acid sequence with one or more sequencing primers or capture primers, performing nucleic acid sequencing, and detecting the predetermined nucleic acid sequence in the sequencing results, thereby detecting the non-nucleic acid biomolecule by nucleic acid sequencing.

35. The method of claim 34, wherein the non-nucleic acid biomolecule is an antibody or antigen-binding fragment thereof.

36. The method of claim 34, wherein the predetermined nucleic acid sequence and non-nucleic acid biomolecule are associated by biotin-avidin binding.

37. The method of claim 34, wherein one or more capture primers are used.

38-40. (canceled)
